(19) United States
(12) Patent Application Publication
(10) Pub. No.: US 2010/0068211 A1
(43) Pub. Date: Mar. 18, 2010

(54) USE OF ANTIRESORPTIVE COMPOUNDS TO PREVENT IONIZING RADIATION-INDUCED ACTIVATION OF OSTEOCLASTS AND RESULTING BONE LOSS

(76) Inventors: Ted A. Bateman, Seneca, SC (US); Jeffrey S. Willey, Clemson, SC (US)

Correspondence Address: Ballard Spahr LLP, SUITE 1000, 999 PEACHTREE STREET, ATLANTA, GA, 30309-3915, US

(21) Appl. No.: 12/368,181

(22) Filed: Feb. 9, 2009

Related U.S. Application Data
(60) Provisional application No. 61/065,072, filed on Feb. 8, 2008.

(51) Int. Cl. A61K 39/395; A61K 31/66; A61K 31/675; A61P 35/00; A61K 31/4535; A61K 38/23; A61K 31/59

(52) U.S. Cl. 424/142.1; 514/108; 514/89; 514/94; 514/324; 514/12; 514/167

(57) ABSTRACT

A method of preventing or treating ionizing radiation-associated loss of bone mass, bone density or bone strength in a subject is provided, comprising administering to the subject an amount of an antiresorptive or osteoclast inhibiting compound sufficient to prevent or mitigate loss of bone mass, density or strength. A method of preventing or treating radiation-associated increase in the number or activity of osteoclasts in a subject is also provided, comprising administering to the subject an amount of an antiresorptive compound sufficient to reduce osteoclast numbers or reduce osteoclast activity and prevent resulting loss of bone mass, density or strength.

### CROSS-REFERENCE TO RELATED APPLICATIONS

This application claims the benefit of U.S. Provisional Application No. 61/065,072, filed Feb. 8, 2008, which application is incorporated herein in its entirety.

### STATEMENT REGARDING FEDERALLY SPONSORED RESEARCH

This invention was made with government support under grants awarded by the National Space Biomedical Research Institute through NASA grant NCC 9-58, and the National Institute of Arthritis, Musculoskeletal and Skin Disorders grant number R21AR054889. The government has certain rights in the invention.

### BACKGROUND

Approximately 1.4 million new cases of cancer are diagnosed each year, nearly half of which use radiation therapy as a treatment [National Cancer Society]. Radiotherapy regimens and standards of care may vary. The variables can include total dose to the tumor that generally ranges from 40-100 Gy; dose deposited to healthy tissue varies with stereotactic techniques and for each treatment plan; and radiation types are generally photons (x-rays and gamma rays) and electrons, but can include protons, helium nuclei and at very specialized clinical locations neutrons and heavy ions (e.g. carbon).

Regardless of the dose, plan and source used for any given patient, healthy, normal (non tumor) tissues inevitably receive significant doses of radiation. For many types of cancer, bone is one of these normal (non-tumor) tissues that absorbs radiation during therapy. As survival rates among cancer patients increase, secondary effects from treatment, including the effects of radiation on normal tissue, are more of a concern.

The types of cancers where normal bone (e.g., structurally important components of the skeletal system such as vertebra, hip, pelvis, ribs and proximal femur) is likely to receive doses of radiation include colon, rectal, anal, cervical, uterus, ovary, urinary/bladder, prostate, breast, stomach, esophagus, lung, and brachial. Additionally, patients requiring bone marrow transplantation (e.g. for leukemia and lymphatic cancers) may receive whole body irradiation.

An estimated 450,000 new cases of pelvic cancers occurred in 2007 (colorectal: 150,000; cervical/uterus/ovary: 65,000; urinary/bladder: 65,000; and prostate: 200,000) [National Cancer Society]. An exemplary pelvic tumor regimen includes the following: 54 gray (Gy) Total: 1.8 Gy Fractions; 30 Fractions for 6 weeks. In this regimen each hip (including the pelvis, proximal femur, and femoral neck) can receive approximately 25-27 Gy. Dose is measured in terms of energy per unit mass (Gy=Joules/kilogram). Additionally, approximately 215,000 new cases of lung cancer near the vertebra occurred in 2007. More than 165,000 women developed breast cancer, increasing rates of rib fracture. An estimated 100,000 men and women developed leukemia or lymphatic tumors in 2007. With an aging population surviving cancer treatment, the increased incidence of fractures from these irradiated skeletal elements may substantially reduce quality of life.

The skeleton is a dynamic organ system that is constantly undergoing replacement of bone (remodeling) to maintain structural strength and competency. This includes the breakdown (resorption) of bone by osteoclast cells, and the synthesis (formation) of bone by osteoblast cells. As defined by the World Health Organization, “osteoporosis is a disease characterized by low bone mass and structural deterioration of bone tissue, leading to bone fragility and an increased susceptibility to fractures, especially of the hip, vertebra, and wrist. Osteoporosis occurs primarily as a result of normal aging, but can arise as a result of impaired development of peak bone mass (e.g. due to delayed puberty or malnutrition) or excessive bone loss during adulthood (e.g. due to estrogen deficiency in women, undernutrition, or corticosteriod use).”

There are existing therapies to treat osteoporosis that act predominantly (but not exclusively) by inhibiting the action of osteoclast cells. These therapies are generally referred to as antiresorptive.

Bisphosphonates are a common therapy for osteoporosis. Bisphosphonates induce osteoclast apoptosis, inhibit osteoclastogenesis, and impair the resorption process, thereby decreasing bone resorption and reducing the rate of bone remodeling. Bisphosphonates have a high binding affinity for the calcium phosphate present within the hydroxyapatite of bone. Thus, they will bind to the bone surface. Mineral is resorbed underneath the osteoclast due to hydrogen ions pumped out into the space between the osteoclast and bone.

Bisphosphonates are released from the mineral and endocytosed by the resorbing cell. Once bound, these drugs function to reduce osteoclast number and activity, decreasing bone resorption. As a result of diminishing the number of osteoclasts, the number of new bone modeling units are also decreased, which is necessary for resorption of older bone and ultimately formation of newer bone. Thus both resorption and formation (collectively termed “turnover”) are reduced (Fleisch, 2000). These agents can then directly impact the osteoclast, reducing activity and number.

Bisphosphonates have been identified and approved for human use. These include risedronate, zoledronate, ibandronate, aledronate, and pamidronate (bisphosphonates not containing nitrogen have been approved for clinical use: etidronate and tiludronate). The chemical structures of these compounds have been disclosed (Fleisch, 2000). Nitrogen-containing bisphosphonates inhibit the mevalonate pathway (production of cholesterol and isoprenoid lipids) by preventing the formation of farnesyl diphosphate synthase. The synthesis of geranylgeranyl pyrophosphate and farnesyl pyrophosphate is inhibited, thus suppressing lipid modification of several GTPases following translation. These proteins include Ras, Rac, Rho, Rab. The functions of these proteins include regulation of osteoclast morphology including the production of the ruffled membrane required for efficient resorption of bone (increases surface area while secreting H+ ions and proteases), regulating apoptosis, and cytoskeletal rearrangement. By disrupting the normal concentrations of these proteins, resorptive function of the osteoclast is reduced and the apoptosis rate of osteoclasts increases:

Osteoprotegerin (OPG), a member of the tumor necrosis factor receptor superfamily, competes with RANK/RANKL binding as a soluble decoy receptor for RANKL, blocking the pathway (Kostenuik and Shalhoub, 2001; Simonet et al., 1997). This RANKL blocking compound can be but is not limited to the protein, or a variation/modification of the protein, (OPG), an antibody to RANKL (denosumab), any other decoy receptor for RANKL or a compound that binds to RANK without activating the nuclear factor-κB ligand pathway.

Additionally, therapies that have been used to treat or prevent bone include, but are not limited to selective estrogen receptor modulators (SERMs), and calcitonin compounds.

The present data show for the first time that exposure to ionizing radiation for the treatment of cancers is a cause for osteoclast activation leading to excessive bone loss. Based on the present teaching, it is recognized that radiation-induced bone loss can be treated with and prevented or mitigated by current and future therapies that inhibit osteoclast activity.

### SUMMARY

The invention relates to a method of preventing or treating ionizing radiation-associated loss of bone mass, bone density or bone strength in a subject, comprising administering to the subject an amount of an antiresorptive or osteoclast inhibiting compound sufficient to prevent or mitigate loss of bone mass, density or strength.

Also provided is a method of preventing loss of bone mass, density or strength in patients receiving or about to receive radiation therapy, comprising administering to the subject an amount of an antiresorptive compound sufficient to prevent loss of bone mass and/or bone density.

A method of preventing or treating radiation-associated increase in the number or activity of osteoclasts in a subject is also provided, comprising administering to the subject an amount of an antiresorptive compound sufficient to reduce osteoclast numbers or reduce osteoclast activity.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 presents graphs comparing serum concentrations of TRAP5b (left) and osteocalcin (right) from non-irradiated and 2 Gy whole-body irradiated mice one day after irradiation. (*) P<0.05 following t-test.

FIG. 2 presents graphs comparing serum concentrations of TRAP5b (left) and osteocalcin (right) from non-irradiated and 2 Gy whole-body irradiated mice three day after irradiation. (*) P<0.05 following t-test.

FIG. 3 shows multinucleated, TRAP+ cells from marrow harvested one day after 2 Gy whole-body irradiation from mice femora and cultured on chamber slides for one week. TRAP⁺ cells appear reddish-brown, nuclei are counterstained with hemotoxylin. Arrows indicate multinucleated, TRAP⁺ cells. (A) Non-irradiated (control); (B) 2 Gy irradiated; (C) histogram comparing osteoclast numbers from non-irradiated (left) and irradiated (right) cultures. Bars represent standard error of the mean. (*) P<0.05 following repeated measures ANOVA comparing replicates from each sample between groups.

FIG. 4 shows increased osteoclast surface and numbers 3-days after irradiation. Representative images after tartrate-resistant acid phosphatase (TRAP) staining within the proximal metaphysis from (panel A) nonirradiated control mice and (panel B) mice irradiated with 2 Gy 3 days previously. Original magnification 400×. Sections were stained to indicate the presence of red-colored TRAP+ osteoclasts along the trabecular surfaces (indicated by arrows). For the histomorphometric variables determined from these sections, values were quantified within the secondary spongiosa, extending 0.5 mm distal from the primary spongiosa. Panel C: Osteoclast surface as a percentage of total bone surface (Oc.S/BS; %), eroded surface with the exclusion of osteoclast surface [ES(Oc−)BS]; and eroded surface with the inclusion of osteoclast surface [ES(Oc+)BS]. Panel D: The number of osteoclasts (N.Oc/BS) as a percentage of total bone surface. Error bars represent SEM. ^{a P<}0.001 and ^{b} P<0.05, after t test.

FIG. 5 shows example of finite element mesh generated from CT scan of a patients proximal femur. Finite element analysis is used to computationally test the strength of the bone in two loading conditions: 1) single leg stance, and 2) falling load on the hip.

FIG. 6 shows that a 2, 4 and 6 Gy dose of whole body X-rays caused approximately the same amount of bone loss. 2 Gy is the minimum dose that elicits the maximum amount of trabecular bone loss in mice. Black represents non-irradiated control mice and white bars represent irradiated mice.

FIG. 7 shows that single limb or whole body exposure to X-rays causes the same amount of bone loss indicating that radiation-induced bone loss is a local response. The dose response examination from Example 3a is confirmed with local exposure. Black represents non-irradiated control mice and white bars represent irradiated mice.

FIG. 8 shows that radiation-induced bone loss is very rapid. There is the same amount of loss 1-week after exposure compared to 2-weeks after exposure. Black represents non-irradiated control mice and white bars represent irradiated mice.

FIG. 9 shows that radiation causes loss of trabecular volume fraction and connectivity density independent of sex. Black represents non-irradiated control mice and white bars represent irradiated mice.

FIG. 10 shows that radiation causes a loss of bone mass in both growing and skeletally mature mice. Black represents non-irradiated control mice and white bars represent irradiated mice.

FIG. 11 shows that radiation causes a rapid decline in bone mass at the proximal tibia of mice exposed to 2 Gy whole body x-rays, with a majority of the loss occurring within the first week after exposure. 105 micrograms/kg/week completely prevents this loss of bone mass.

FIG. 12 shows that risedronate prevents rapid radiation-induced bone loss at the distal femur.

FIG. 13 shows that risedronate prevents rapid radiation-induced bone loss at the 5^{th} lumbar vertebra.

FIG. 14 shows that TRAP5b, a marker for osteoclast activity was elevated 7 days after exposure in IR+Plac treated mice. Risedronate reduced TRAP5b levels at all time points, even after irradiation.

FIG. 15 is a histological analysis of proximal tibia trabecular bone showing a greater osteoclast surface in both IRR+Placebo and IRR+Risedronate treated mice one week, but not two and three weeks, after exposure. The increase in osteoclast surface, even with risedronate treatment indicates that antiresorptive doses may need to be high and may need to preceed radiation exposure. Black represents non-irradiated control mice treated with placebo, white bars represent irradiated mice treated with placebo and grey bars represent irradiated mice treated with 30 micrograms/kg risedronate every other day.

FIG. 16. A 10 mg/kg dose of zoledronate prevents radiation-induced bone loss. Black represents non-irradiated control mice and white bars represent irradiated mice.

FIG. 17 shows that radiation causes loss of trabecular volume fraction and connectivity density in more than one strain of mouse, it is not specific to B6 mice. DBA/2 mice are also susceptible to radiation-induced bone loss to the same approximate degree. Black represents non-irradiated control mice and white bars represent irradiated mice.

FIG. 18 shows that a marrow ablating dose of gamma-rays causes a rapid loss of trabecular bone. Black represents non-irradiated control mice and white bars represent irradiated mice.

FIG. 19 is a histological analysis of osteoclast and osteoblast surfaces indicating no significant differences, showing that the process of bone loss is largely complete two weeks after exposure. Black represents non-irradiated control mice and white bars represent irradiated mice.

FIG. 20 shows a change in trabecular volume fraction (BV/TV) and connectivity density (Conn.Den.) for multiple radiation types results in a long-term decline of 29% to 39%. Black represents non-irradiated control mice and white bars represent irradiated mice.

FIG. 21 shows that normally loaded and hindlimb unloaded mice have approximately the same relative amount of radiation-induced bone loss, even with the large disuse mediated bone loss of approximately 75%. Black represents non-irradiated control mice and white bars represent irradiated mice.

FIG. 22 shows that mice with the gene for interleukin-1 receptor knocked out are not spared from radiation-induced bone loss. Black represents non-irradiated control mice and white bars represent irradiated mice.

FIG. 23 shows that mice with the gene for interleukin-6 knocked out are not spared from radiation-induced bone loss. Black represents non-irradiated control mice and white bars represent irradiated mice.

### DETAILED DESCRIPTION OF THE INVENTION

### Definitions

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents.

It must be noted that as used herein and in the appended claims, the singular forms “a,” “an,” and “the” include plural referents unless the context clearly dictates otherwise. Thus, for example, reference to “a cell” includes a plurality of such cells; reference to “the antiresporptive” is a reference to one or more antiresorptive compounds and homologs or functional equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

The term “preventing” as used herein refers to administering a compound prior to the onset of clinical symptoms of a disease or conditions so as to prevent or reduce the severity of a physical manifestation of aberrations associated with the disease or condition.

The term “treating” as used herein refers to administering a compound after the onset of clinical symptoms. Treating can include a partial improvement in symptoms (e.g., a reduction bone loss or an increase in bone density), or may be a complete cessation of symptoms (e.g., complete normalization of bone mass, density and structure). The term “in need of treatment” as used herein refers to a judgment made by a caregiver (e.g. physician, physician's assistant, nurse, or nurse practitioner in the case of humans; veterinarian in the case of animals, including non-human mammals) that an individual or animal requires or will benefit from treatment. This judgment is made based on a variety of factors that are in the realm of a care giver's expertise, but that includes the knowledge that the individual or animal is ill, or will be ill, as the result of a condition that is treatable by the compounds of the invention. A similar judgment may be made by a caregiver to determine if a subject (individual) is “in need of prevention.”

The terms “individual” and “subject” as used herein refer to a mammal, including animals, preferably mice, rats, other rodents, rabbits, dogs, cats, swine, cattle, sheep, horses, or primates, particularly humans. The human can be an adult, an adolescent or a child.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

As used herein, radiation is defined as ionizing radiation that can be of the following types: photons, electrons, protons and heavy ions that have enough energy to ionize an atom. As used herein, radiation does not refer to non-ionizing types of radiation such as ultraviolet radiation, visible light, near infrared radiation, far infrared radiation, microwaves or radio waves.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Treatment/Prevention Methods

Provided is a method of preventing or treating radiation-associated (also referred to herein as “radiation-induced”) loss of bone mass, bone density or bone strength in a subject, comprising administering to the subject an amount of an antiresorptive or osteoclast inhibiting compound sufficient to prevent or mitigate loss of bone mass, density or strength. Radiation-induced bone loss is loss that represents a decline in volumetric bone mineral density (vBMD) or volumetric bone mineral content (vBMC) of at least at least 5% from pre-treatment as measured by QCT at any skeletal site in the radiotherapy treatment region during the peri-radiation period.

Bone density, or bone mineral density (BMD), is the common parameter used for identifying an osteoporotic condition. However, a subject can lose bone mass without necessarily losing bone density. Bone density and bone mass are clinically quantifiable parameters and bone strength can be approximately calculated with computational tools.

The methods for determining BMD are: DXA (Dual Energy X-ray Absorptiometry); pDXA (Peripheral Dual Energy X-ray Absorptiometry); SXA (single Energy X-ray Absorptiometry); QUS (Quantitative Ultrasound); QCT (Quantitative Computed Tomography); pQCT (Peripheral Quantitative Computed Tomography); RA (Radiographic Absorptiometry) [National Osteoporosis Foundation].

The present data indicate that osteoclasts are significantly activated by ionizing radiation, which is the cause of substantial and rapid bone loss. The data also indicate that with the administration of drugs commonly used for treatment of osteoporosis, this effect is significantly mitigated. Radiation-induced activation of osteoclasts is represented by an increase in osteoclast number, osteoclast surface, osteoclast number normalized to bone surface, or osteoclast surface normalized to bone surface of 20% or greater at any skeletal site in the radiotherapy treatment region during the per-radiation period.

Effectiveness of the antiresorptive is demonstrated, for example, by a reduction in radiation-induced bone loss or osteoclast activation by approximately 20%. For example, vBMD or vBMC declines within the radiotherapy treatment region are reduced by at least 5% to 4% in a given patient during the peri-radiation period. In this example, osteoclast number, osteoclast surface, osteoclast number normalized to bone surface, or osteoclast surface normalized to bone surface somewhere within the radiotherapy treatment field (area) is reduced from 20% to 16% (i.e., a reduction vBMD or vBMC by 4% and a reduction in bone loss or osteoclast activity of 20% (4÷20)) in a given patient during the peri-radiaiton period. It will be recognized that the reduction in radiation-induced bone loss will differ at different sites within a given patient. It will also be recognized that larger reductions in bone loss can be experienced, for example, 6%, 7%, 8%, 9%, 10%, 11%, 12%, 13%, 14%, 15%, 16%, 17%, 18%, 19%, 20%, etc.

As used herein an “antiresorptive” compound or agent is an agent that prevents or reduces bone resorption, and is synonymous with an “osteoclast inhibiting” compound, an agent that reduces the differentiation, development, number, activation, activity or survival of osteoclasts. As used herein an “osteoclast” is a bone resorbing/removing cell.

As used herein, the “amount” of antiresorptive, can be described in terms of amount of a given dose, amount based on frequency or total amount, which results from the combination of dose amount and frequency of dosing.

Also provided is a method of preventing or treating radiation-induced loss of bone mass, density or strength in a patient receiving or about to receive radiation therapy, comprising administering to the subject an amount of an antiresorptive compound sufficient to prevent loss of bone mass and/or bone density and/or bone strength caused by osteoclast activation. In the method of preventing loss of bone mass, density or strength in a patient receiving or about to receive radiation therapy, the patient can be a patient diagnosed with cancer. The patient can be newly diagnosed with cancer or newly diagnosed with a relapse of previously treated cancer, for example by radiological tools such as mammogram, biopsy, blood or urine test or other method of diagnosis. Thus, provided is a method of preventing or treating radiation-associated loss of bone mass, bone density or bone strength in a subject who has been diagnosed with cancer, but who has not yet been treated by radiation therapy for cancer. The cancer can be any cancer for which radiation therapy would be applicable. For example, the method can be used to prevent or treat radiation-induced loss of bone mass, density or strength in a patient diagnosed with pelvic cancers (e.g., colorectal, cervical, uterine, ovarian, urinary/bladder, testicular and prostate, lung cancer, breast cancer, leukemia or lymphatic cancer). The present treatment with an antiresorptive is also an important component of maintaining skeletal competency for patients receiving whole body radiation for bone marrow transplantation for cancers such as leukemia and lymphoma or other conditions. Subjects receiving radiation therapy after being diagnosed with any of other cancers disclosed herein can also be treated by the present method.

A method of preventing or treating radiation-associated increase in the number or activity of osteoclasts in a subject is also provided, comprising administering to the subject an amount of an antiresorptive compound sufficient to reduce osteoclast numbers or reduce osteoclast activity. The reduction in osteoclast activity or number has the effect of reducing or preventing loss of bone mass, density or strength. The method can be accomplished by administering an osteoclast inhibiting compound, for example, the antiresorptive agents in the dosing regimens disclosed herein. The number of osteoclasts is not typically measured in the clinical setting, except by biopsy, and it is expected to be rare for a cancer patient have a bone biopsy. However, it is shown herein that radiation induces an increase in osteoclast numbers and/or activity, and it is recognized by those in this field that the administration of antiresorptive or osteoclast inhibiting compounds either reduces osteoclast number or reduces osteoclast activity. Thus, provided is a method of preventing loss of bone mass, density or strength in a patient identified as having a radiation-induced increase number or activity of osteoclasts. Similarly, provided is a method of preventing loss of bone mass, density or strength in a patient diagnosed as having a radiation-induced increase number or activity of osteoclasts.

The present method of preventing or treating radiation-associated loss of bone mass, density or strength in a subject, can involve any form of radiation therapy approved for cancer radiotherapy in a patient diagnosed with cancer. Examples of radiation therapy regimens are well known in the scientific and medical literature. Descriptions of examples of such radiation therapy, including external, internal (brachytherapy), etc., are described in Example 7 and elsewhere in the present application.

### Compositions for Use in the Methods

In the methods of preventing or reducing loss of bone density, mass or strength or of reducing osteoclast number or activity, the antiresorptive compound can be selected from known or later developed antiresorptive compounds, including the compounds disclosed herein. For example, the antiresorptive compound can be a bisphosphonate. See section on bisphosphonates below.

In the methods of preventing or reducing loss of bone density, mass or strength, or of reducing osteoclast number or activity, of particular value as the antiresorptive compound are denosumab, risedronate, alendronate, zoledronate, pamidronate and ibandronate.

In the methods of preventing or reducing loss of bone density, mass or strength, or of reducing osteoclast number or activity, the antiresorptive compound can be selected from the group consisting of bisphosphonates. Bisphosphonates are known to induce osteoclast apoptosis, inhibit osteoclastogenesis and reduce osteoclast activity. The class of bisphosphonates includes, for example, alendronate, risedronate, ibandronate, zoledronate, pamidronate, etidronate and tiludronate. The chemical structure and formula for and a method of making each of these compounds is known in the literature.

In the methods of preventing or reducing loss of bone density, mass or strength, or of reducing osteoclast number or activity, the antiresorptive compound can be selected from the group consisting of anti-RANKL compounds. These are compounds that block the activity of RANKL (receptor activator of nuclear factor-κB ligand) by preventing the binding of the RANK ligand protein (membrane bound or soluble forms) to the RANK protein receptor on the osteoclast. Compounds that block RANKL activity block the differentiation, development, activity, activation and/or survival of osteoclasts or promote osteoclast apoptosis by preventing the binding of the RANK ligand protein (membrane bound or soluble forms) to the RANK protein receptor on the osteoclast. Anti-RANKL compounds include, for example, denosumab (a fully human anti-RANKL antibody that binds RANK ligand and blocks its binding to RANK, formerly AMG162 (Amgen). See, for example, (Body et al., 2006; Lewiecki et al., 2007; Lipton et al., 2007; McClung et al., 2006), which are incorporated herein by reference for their teaching of the composition of denosumab and its uses.), osteoprotegerin (OPG) (a TNF receptor family member that binds RANKL and thereby prevents activation of RANK (Simonet et al., 1997), portions of the OPG protein, decoy receptor for RANKL or a compound that binds to RANK without activating the nuclear factor-κB ligand pathway.

In the methods of preventing or reducing loss of bone density, mass or strength, or of reducing osteoclast number or activity, the antiresorptive compound can be selected from the group consisting of estrogen blocking, or selective estrogen receptor modulator (SERM) compounds. SERM compounds include, for example EVISTA® (raloxifene). The systematic (IUPAC) name for EVISTA® is [6-hydroxy-2-(4-hydroxyphenyl)-benzothiophen-3-yl]-[4-[2-(1-piperidyl)ethoxy]phenyl]-methanone. Identifiers include CAS number: 84449-90-1; ATC code: G03XC₀₁; PubChem: 5035; DrugBank: APRD00400. The chemical formula is C₂₈H₂₇NO₄S and the Mol. mass is 473.584 g/mol. In the methods of preventing or reducing loss of bone density, mass or strength, or of reducing osteoclast number or activity, the antiresorptive compound can be selected from the group consisting of calcitonin compounds. Calcitonin is a 32-amino acid polypeptide hormone (calcitonin/calcitonin-related polypeptide, alpha; identifiers include symbol CALCA and alt. symbol CALC₁; databases disclosing calcitonin include Entrez: 796; HUGO: 1437; OMIM: 114130; RefSeq: NM_{—}001741; UniProt: P01258. The locus is Chr. 11 p15.4).

In the methods of preventing or reducing loss of bone density, mass or strength, or of reducing osteoclast number or activity, the antiresorptive compound can be administered in conjunction with calcitriol (1,25-dihydroxycholecalciferol) or calcium supplements.

Based on the disclosure herein, it is recognized that other antiresorptive compounds, not specifically identified herein, are useful in the present methods and compositions. Likewise, if modifications to existing antiresorptives are developed, they can be used in the present methods to the same extent as the disclosed molecule is.

### Bisphosphonates

The bisphosphonates that can be used in the disclosed compositions have the formula:

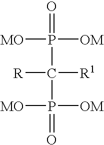

wherein R is a unit having the formula:

-(L¹)ₓ-Z

Z and L¹ and the index x are further defined herein;
R¹ is further defined herein; and
M represents hydrogen or a pharmaceutically acceptable cation capable of providing electronic neutrality to the molecule. In one embodiment M is a cation having a charge of +1 wherein the bisphosphonate has the above formula. In another embodiment, M is a cation having the charge of +2 wherein the bisphosphonate can be represented by the formula:

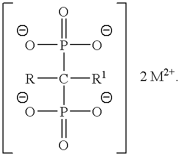

The following are non-limiting examples of cations that can form salts ammonium, sodium, lithium, potassium, calcium, magnesium, bismuth, and the like.

### Z Units

Z is a unit chosen from:
i) C₁-C₁₂ substituted or unsubstituted linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) C₆ or C₁₀ substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) C₁-C₉ substituted or unsubstituted heterocyclic as further defined herein;
iv) C₁-C₁₁ substituted or unsubstituted heteroaryl as further defined herein;
v) —[C(R^{2a})(R^{2b})]_{y}OR³;
   a) wherein R³ is chosen from:
   b) —H;
   c) C₁-C₁₂ substituted or unsubstituted linear, branched, or cyclic alkyl;
   d) C₆ or C₁₀ substituted or unsubstituted aryl or alkylenearyl;
   e) C₁-C₉ substituted or unsubstituted heterocyclic;
   f) C₁-C₁₁ substituted or unsubstituted heteroaryl;
      for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃
vi) —[C(R^{2a})(R^{2b})]_{y}N(R^{4a})(R^{4b});
   a) wherein R^{4a} and R^{4b} are each independently chosen from:
      i) —H;
      ii) —OR⁵;
      R⁵ is hydrogen or C₁-C₄ linear alkyl;
   b) C₁-C₁₂ substituted or unsubstituted linear, branched, or cyclic alkyl;
   c) C₆ or C₁₀ substituted or unsubstituted aryl;
   d) C₁-C₉ substituted or unsubstituted heterocyclic;
   e) C₁-C₁₁ substituted or unsubstituted heteroaryl; or
   f) R^{4a} and R^{4b} can be taken together to form a substituted or unsubstituted ring having from 3 to 10 carbon atoms and from 0 to 3 heteroatoms chosen from oxygen, nitrogen, and sulfur;
vii) —[C(R^{2a})(R^{2b})]_{y}C(O)R⁶;
   a) wherein R⁶ is chosen from:
      i) C₁-C₁₂ substituted or unsubstituted linear, branched, or cyclic alkyl;
      ii) —OR⁷;
      R⁷ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   b) —N(R^{8a})(R^{8b}); and
      R^{8a} and R^{8b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{8a} and R^{8b} can be taken together to form a substituted or unsubstituted ring having from 3 to 10 carbon atoms and from 0 to 3 heteroatoms chosen from oxygen, nitrogen, and sulfur;
viii) —[C(R^{2a})(R^{2b})]_{y}OC(O)R⁹;
   wherein R⁹ is chosen from:
   a) C₁-C₁₂ substituted or unsubstituted linear, branched, or cyclic alkyl;
   b) —N(R^{10a})(R^{10b}); and
      R^{10a} and R^{10b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{10b} can be taken together to form a substituted or unsubstituted ring having from 3 to 10 carbon atoms and from 0 to 3 heteroatoms chosen from oxygen, nitrogen, and sulfur;
ix) —[C(R^{2a})(R^{2b})]_{y}NR¹¹C(O)R¹²;
   wherein R¹¹ is chosen from:
   a) —H; and
   b) C₁-C₄ substituted or unsubstituted linear, branched, or cyclic alkyl;
   c) wherein R¹² is chosen from:
      i) C₁-C₁₂ substituted or unsubstituted linear, branched, or cyclic alkyl; and
      ii) —N(R^{13a})(R^{13b});
      R^{13a} and R^{13b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{13a} and R^{13b} can be taken together to form a substituted or unsubstituted ring having from 3 to 10 carbon atoms and from 0 to 3 heteroatoms chosen from oxygen, nitrogen, and sulfur;
x) —[C(R^{2a})(R^{2b})]_{y}CN;
xi) —[C(R^{2a})(R^{2b})]_{y}NO₂;
xii) —[C(R^{2a})(R^{2b})]_{y}SO₂R¹⁴;
   wherein R¹⁴ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
xiii) halogen; and
xiv) —SR¹⁵;
   R¹⁵ is chosen from:
   i) C₁-C₁₂ substituted or unsubstituted linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
   ii) C₆ or C₁₀ substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
      R^{2a} and R^{2b} are each independently hydrogen or C₁-C₄ alkyl; and
      the index y is from 0 to 5.

In one embodiment R can be chosen from:
i) substituted or unsubstituted C₃-C₇ carbocyclic rings;
ii) substituted or unsubstituted C₁-C₉ heteroaryl rings;
iii) substituted or unsubstituted C₁-C₉ heterocyclic rings; or
v) substituted or unsubstituted phenyl.

In some embodiments Z can be a substituted or unsubstituted C₁, C₂, C₃, or C₄ heteroaryl or heterocyclic 5-member ring/, Non-limiting examples of Z units which can be independently chosen from:
i) a pyrrolidinyl ring having the formula;

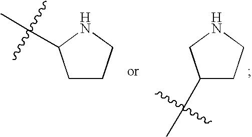

ii) a pyrrolyl ring having the formula:

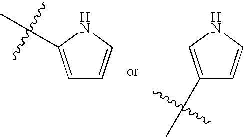

iii) a 4,5-dihydroimidazolyl ring having the formula:

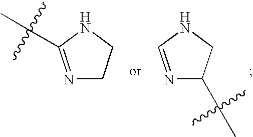

iv) a pyrazolyl ring having the formula:

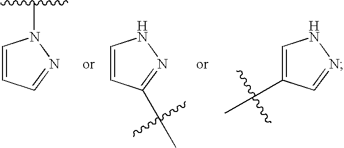

v) an imidazolyl ring having the formula:

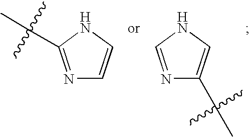

vi) a [1,2,3]triazolyl ring having the formula:

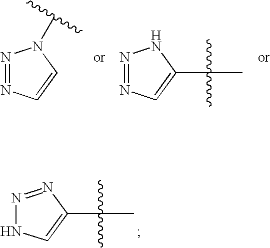

vii) a [1,2,4]triazolyl ring having the formula:

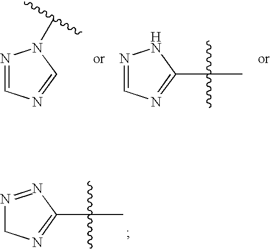

viii) tetrazolyl ring having the formula:

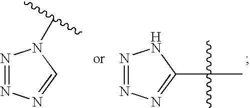

ix) a [1,3,4] or [1,2,4]oxadiazolyl ring having the formula:

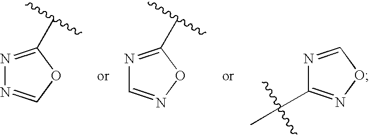

x) a pyrrolidinonyl ring having the formula:

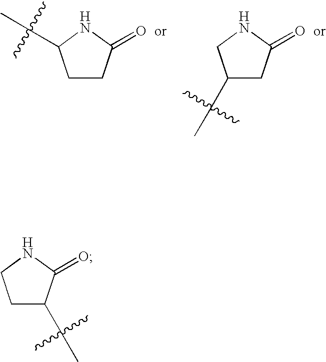

xi) an imidazolidinonyl ring having the formula:

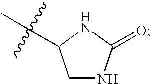

xii) an imidazol-2-only ring having the formula:

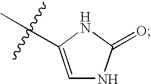

xiii) an oxazolyl ring having the formula:

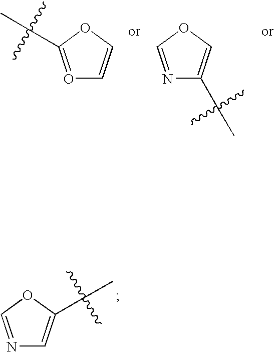

xiv) an isoxazolyl ring having the formula:

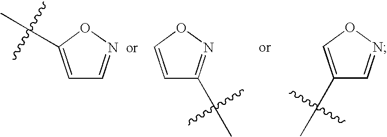

xv) a dihydrothiazolyl ring having the formula:

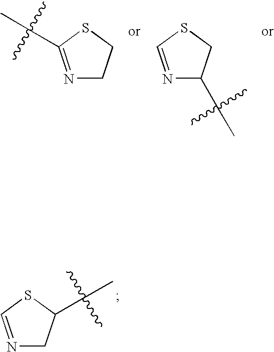

xvi) a furanly ring having the formula:

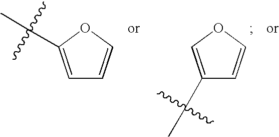

xvii) a thiophenyl having the formula:

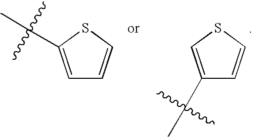

Non-limiting examples of units which can substitute for one or more hydrogen ring atoms of the C₂, C₃, or C₄ heteroaryl or heterocyclic 5-member ring can be independently chosen from:
a) C₁-C₄ linear or branched alkyl;
b) C₁-C₄ linear or branched alkoxy;
c) —C(O)OR¹³; or
d) —SO₂NR^{14a}R^{14b};
   wherein R¹³, R^{14a}, and R^{14b} are each independently hydrogen, methyl or ethyl.

Non-limiting examples of substituted C₁, C₂, C₃, or C₄ heteroaryl or heterocyclic 5-member rings can include:
i) 3-methylisoxazol-5-yl and 5-methylisoxazol-3-yl having the formulae:

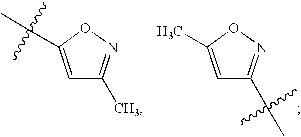

ii) 3-methyl-5-phenylisoxazol-4-yl and 3-phenyl-5-methylisoxazol-4-yl having the formulae:

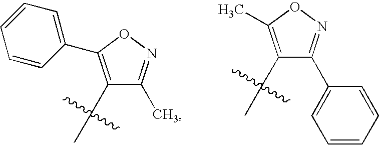

iii) 3,5-dimethylpyrazol-1-yl having the formulae:

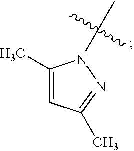

iv) 1-(methylcarboxy)[1,2,3]-triazol-4-yl and 1-(ethylcarboxy)-[1,2,3]triazol-4-yl having the formulae:

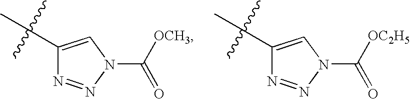

v) 4-(methylcarboxy)[1,2,3]triazol-1-yl and 4-(ethylcarboxy)[1,2,3]triazol-1-yl having the formulae:

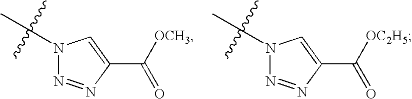

vi) 1-(methylcarboxy)methyl-[1,2,3]triazol-4-yl and 1-(methylcarboxy)-methyl[1,2,3]triazol-1-yl having the formulae:

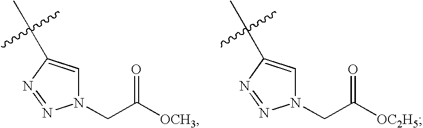

vii) 4-(methylcarboxy)methyl-[1,2,3]triazol-1-yl and 4-(ethylcarboxy)methyl-[1,2,3]triazol-1-yl having the formulae:

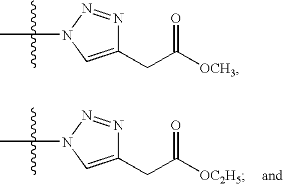

viii) 2-methylpyrrol-1-yl and 3-methylpyrrol-1-yl having the formulae;

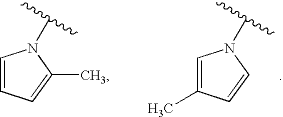

A further embodiment of Z units relates to substituted or unsubstituted C₃, C₄ or C₅ heterocyclic or heteroaryl 6-member rings, non-limiting examples of which can be independently chosen from:
i) a morpholinyl ring having the formula:

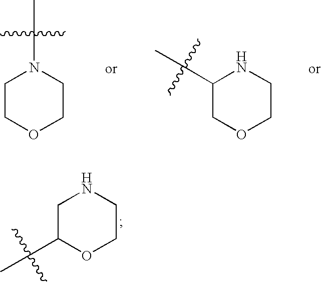

ii) a piperidinyl ring having the formula:

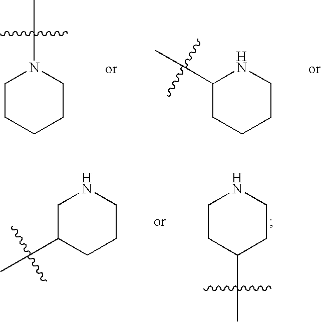

iii) a pyridinyl ring having the formula:

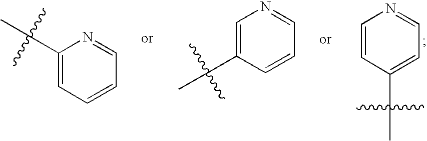

iv) a pyrimidinyl ring having the formula:

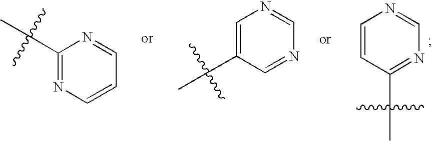

v) a piperazinyl ring having the formula:

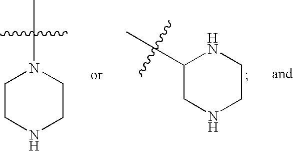

vi) a triazinyl ring having the formula:

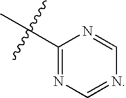

Non-limiting examples of units which can be substituted for one or more hydrogen ring atoms of the C₂, C₃, or C₄ heteroaryl or heterocyclic 6-member ring are independently chosen from:
a) C₁-C₄ linear or branched alkyl;
b) C₁-C₄ linear or branched alkoxy;
c) —C(O)OR¹³; or
d) —SO₂NR^{14a}R^{14b};
   wherein R¹³ and R^{14a}, R^{14b} are each independently hydrogen, methyl or ethyl.

Non-limiting examples of substituted C₃, C₄, or C₅ heteroaryl or heterocyclic 6-member rings include:
i) 4,6-dimethylpyrimidin-2-yl and 4-hydroxy-6-methylpyrimidin-2-yl having the formulae;

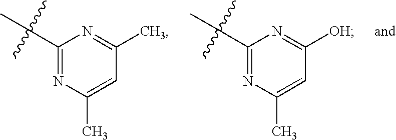

ii) 4-(methylcarboxy)pyridin-2-yl and 4-(ethylcarboxy)pyridin-2-yl having the formulae:

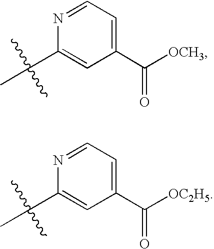

Another related embodiment of Z units relates to substituted or unsubstituted C₇, C₈ or C₉ heterocyclic or heteroaryl fused rings, non-limiting examples of which can be independently chosen from:
i) benzoimidazolyl rings having the formula:

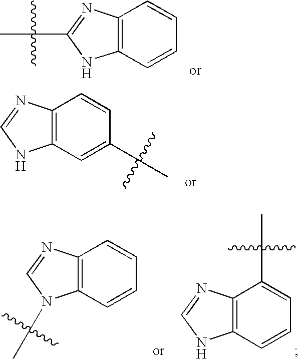

ii) benzothiazolyl rings having the formula:

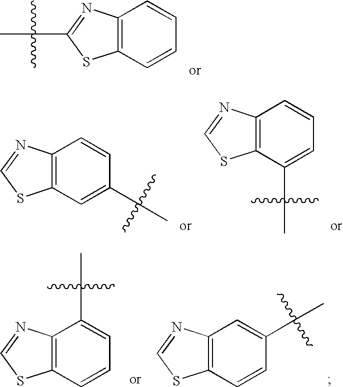

iii) benzoxazolyl rings having the formula:

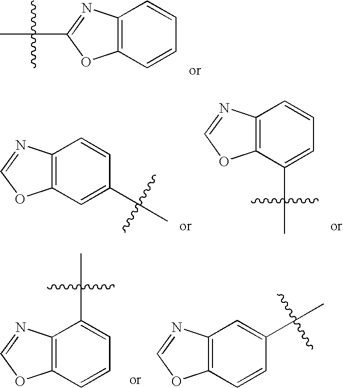

iv) quinazolinyl rings having the formula:

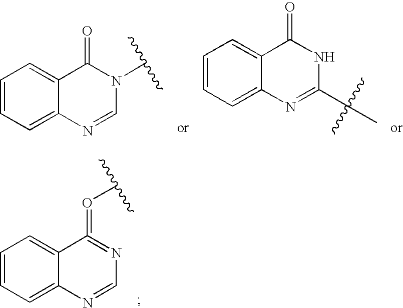

v) 2,3-dihydrobenzo[1,4]dioxinyl rings having the formula:

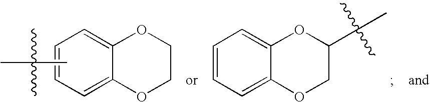

vi) tetrahydroquinolinyl rings having the formula:

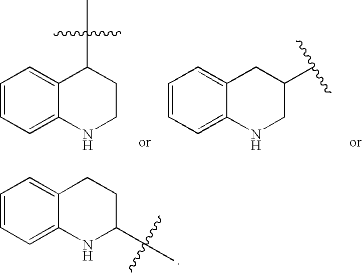

Non-limiting examples of units which can substitute for one or more hydrogen ring atoms of the C₇, C₈, or C₉ heteroaryl or heterocyclic fused rings can be independently chosen from:
a) C₁-C₄ linear or branched alkyl;
b) C₁-C₄ linear or branched alkoxy;
c) —C(O)OR¹³; or
d) —SO₂NR^{14a}R^{14b};
   wherein R¹³, R^{14a}, and R^{14b} can be each independently hydrogen, methyl or ethyl.

Non-limiting examples of substituted C₇, C₈, or C₉ heteroaryl or heterocyclic fused rings include:
i) 2-methylquinazolin-4-yl and 2-methylquinazolinon-3-yl having the formulae:

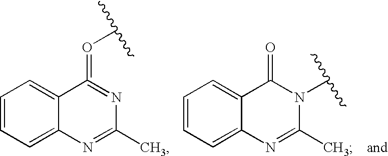

ii) 5-(methylcarboxy)benzothiazol-2-yl and 6-(methylcarboxy)benzothiazol-2-yl having the formulae:

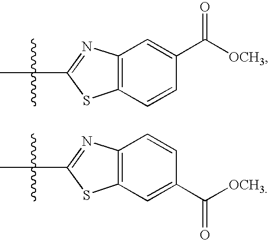

Another related embodiment of Z units relates to substituted or unsubstituted C₃-C₇ carbocyclic rings independently chosen from cyclopropyl (C₃), cyclobutyl (C₄), cyclobutyl (C₄), cyclobutyl (C₄), cyclopentyl (C₅), cyclohexyl (C₆), and cycloheptyl (C₇).

The following embodiment of Z units relates to substituted or unsubstituted phenyl, non-limiting examples of units which can substitute for hydrogen include one or more units independently chosen from:
a) C₁-C₄ linear or branched alkyl; for example, methyl (C₁), ethyl (C₂), n-propyl (C₃), iso-propyl (C₃), n-butyl (C₄), sec-butyl (C₄), iso-butyl (C₄), and tert-butyl (C₄);
b) C₁-C₄ linear or branched alkoxy; for example, methoxy (C₁), ethoxy (C₂), n-propoxy (C₃), iso-propoxy (C₃), n-butoxy (C₄), sec-butoxy (C₄), iso-butoxy (C₄), and tert-butoxy (C₄);
c) —(CH₂)ₜC(O)OR¹³; for example, —CO₂CH₃, —CH₂CO₂CH₃, —CO₂CH₂CH₃, and —CO₂CH₂CH₂CH₃;
d) —(CH₂)ₜOC(O)R¹³; for example, —OCOCH₃, —OCOCH₂CH₃, and —OCOCH₂CH₂CH₃;
e) —(CH₂)ₜC(O)NR^{14a}R^{14b}; for example, —CONH₂, —CONH₂, —CONHCH₃, —(CH₂)CONHCH₃, and —CON(CH₃)₂;
f) —(CH₂)ₜSO₂NR^{14a}R^{14b}; for example, —SO₂NH₂; and —SO₂ NHCH₃;
g) (CHjXₖ)ᵤ; for example, —CH₂F, —CHF₂, and —CF₃;
h) —(CH₂)ₜOH; for example, —OH, and —CH₂OH; or
i) halogen;
   wherein R¹³, R^{14a}, and R^{14b} can be each independently hydrogen, methyl, or ethyl; X is one or more halogen chosen from fluoro, chloro, or iodo; the index j is from 0 to 2; the index k is from 1 to 3; j+k=3; the index t is from 0 to 3; the index u is from 0 to 3.

Non-limiting examples of substituted phenyl units that can be used in preparing the compounds disclosed herein include:

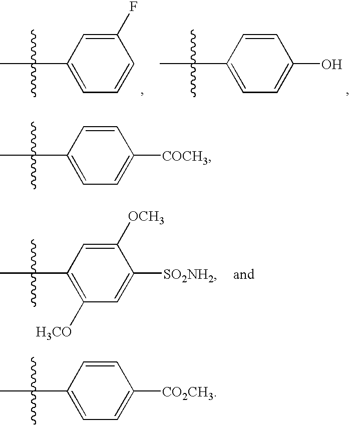

In a yet further embodiment, Z is halogen, for example, fluorine, chlorine, bromine, and iodine.

### L¹ Units

L¹ is a linking unit that when present (the index x=1) serves to connect the Z unit to the tether/linking unit. U is present when the index b is equal to 1 and L¹ is absent when the index x is equal to 0. L¹ is chosen from:
i) —[C(R^{16a}R^{16b})]ₘ—;
ii) OH; or
iii) halogen;
   R^{15a} and R^{16b} are each independently chosen from hydrogen or methyl; and the index m is from 1 to 20.

The first embodiment of L¹ relates to units having the formula:

—(CH₂)ₘ—;

wherein R^{16a} and R^{16b} are both hydrogen. The following are non-limiting examples of this embodiment:

### i) —CH₂—;

### ii) —CH₂CH₂—;

iii) —CH₂CH₂CH₂—;

### iv) —CH₂CH₂CH₂CH₂—;

### v) —CH₂CH₂CH₂CH₂CH₂—;

### vi) —CH₂CH₂CH₂CH₂CH₂CH₂—;

vii) —CH₂CH₂CH₂CH₂CH₂CH₂CH₂—;
viii) —CH₂CH₂CH₂CH₂CH₂CH₂CH₂CH₂—;

### ix) —CH₂CH₂CH₂CH₂CH₂CH₂CH₂CH₂CH₂—; and

x) —CH₂CH₂CH₂CH₂CH₂CH₂CH₂CH₂CH₂CH₂—.

Another first embodiment of L¹ relates to units having the formula:

—(CR^{16a}R^{16b})ₘ—

wherein R^{16a} and R^{16b} can be either hydrogen or methyl. The following are non-limiting examples of this embodiment:

### i) —H₂CH(CH₃)—;

### ii) —H(CH₃)CH₂—;

iii) —H(CH₃)CH₂CH₂—;

### iv) —H₂CH(CH₃)CH₂—;

### v) —H₂CH₂CH(CH₃—);

vi) —H(CH₃)CH(CH₃)CH₂—;

vii) —H₂CH(CH₃)CH(CH₃—);
vii) —H(CH₃)CH₂CH(CH₃)—;

### ix) —H(CH₃)CH₂CH₂CH₂—;

### x) —H₂CH(CH₃)CH₂CH₂—;

### xi) —CH₂CH₂CH(CH₃)CH₂—;

xii) —H₂CH₂CH₂CH(CH₃)—;
xiii) —H(CH₃)CH₂CH₂CH(CH₃)—;
xiv) —H₂CH(CH₂CH₃)—;

### xv) —H(CH₂CH₃)CH₂—;

xvi) —H(CH₂CH₃)CH₂CH₂—;
xvii) —H₂CH(CH₂CH₃)CH₂—;
xviii) —H[CH₂CH(CH₃)₂]CH₂CH₂—;

xix) —H₂CH[CH₂CH(CH₃)₂]CH₂—; and

### xx) —H₂CH₂CH[CH₂CH(CH₃)₂]—.

### R¹ Units

R¹ is a unit chosen from:
i) hydrogen;
ii) OH;
iii) halogen; and
iv) methyl.

In one embodiment, R¹ is —OH. In another embodiment, R¹ is —H. In a further embodiment, R¹ is —Cl.

The following are non-limiting examples of suitable bisphosphonates that can be used as the free acids as shown or as a pharmaceutically acceptable salt thereof.

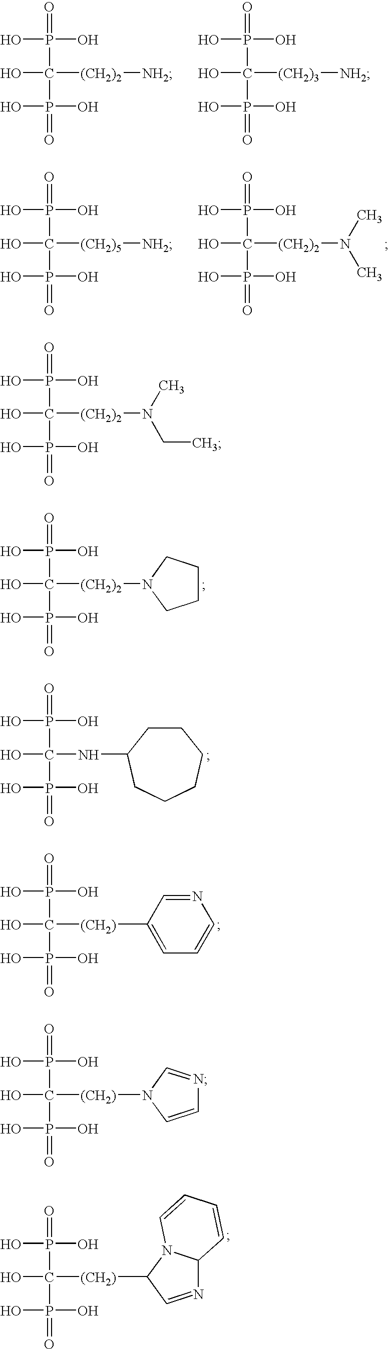

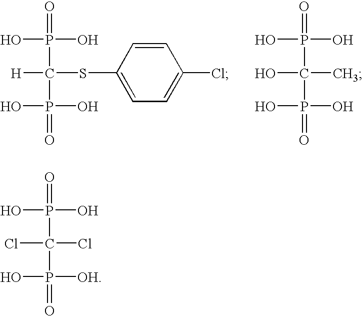

The following chemical hierarchy is used throughout the specification to describe and enable the scope of the present invention and to particularly point out and distinctly claim the units which comprise the compounds of the present invention, however, unless otherwise specifically defined, the terms used herein are the same as those of the artisan of ordinary skill. The term “hydrocarbyl” stands for any carbon atom-based unit (organic molecule), said units optionally containing one or more organic functional group, including inorganic atom comprising salts, inter alia, carboxylate salts, quaternary ammonium salts. Within the broad meaning of the term “hydrocarbyl” are the classes “acyclic hydrocarbyl” and “cyclic hydrocarbyl” which terms are used to divide hydrocarbyl units into cyclic and non-cyclic classes.

As it relates to the following definitions, “cyclic hydrocarbyl” units may comprise only carbon atoms in the ring (carbocyclic and aryl rings) or may comprise one or more heteroatoms in the ring (heterocyclic and heteroaryl). For “carbocyclic” rings the lowest number of carbon atoms in a ring are 3 carbon atoms; cyclopropyl. For “aryl” rings the lowest number of carbon atoms in a ring are 6 carbon atoms; phenyl. For “heterocyclic” rings the lowest number of carbon atoms in a ring is 1 carbon atom; diazirinyl. Ethylene oxide comprises 2 carbon atoms and is a C₂ heterocycle. For “heteroaryl” rings the lowest number of carbon atoms in a ring is 1 carbon atom; 1,2,3,4-tetrazolyl. The following is a non-limiting description of the terms “acyclic hydrocarbyl” and “cyclic hydrocarbyl” as used herein.

A. Substituted and unsubstituted acyclic hydrocarbyl:
For the purposes of the present invention the term “substituted and unsubstituted acyclic hydrocarbyl” encompasses 3 categories of units:
1) linear or branched alkyl, non-limiting examples of which include, methyl (C₁), ethyl (C₂), n-propyl (C₃), iso-propyl (C₃), n-butyl (C₄), sec-butyl (C₄), iso-butyl (C₄), tert-butyl (C₄), and the like; substituted linear or branched alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.
2) linear or branched alkenyl, non-limiting examples of which include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.
3) linear or branched alkynyl, non-limiting examples of which include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₉), 5-hydroxy-5-ethylhept-3-ynyl (C₉), and the like.
   B. Substituted and unsubstituted cyclic hydrocarbyl:
   For the purposes of the present invention the term “substituted and unsubstituted cyclic hydrocarbyl” encompasses 5 categories of units:
1) The term “carbocyclic” is defined herein as “encompassing rings comprising from 3 to 20 carbon atoms, wherein the atoms which comprise said rings are limited to carbon atoms, and further each ring can be independently substituted with one or more moieties capable of replacing one or more hydrogen atoms.” The following are non-limiting examples of “substituted and unsubstituted carbocyclic rings” which encompass the following categories of units:
   i) carbocyclic rings having a single substituted or unsubstituted hydrocarbon ring, non-limiting examples of which include, cyclopropyl (C₃), 2-methyl-cyclopropyl (C₃), cyclopropenyl (C₃), cyclobutyl (C₄), 2,3-dihydroxycyclobutyl (C₄), cyclobutenyl (C₄), cyclopentyl (C₅), cyclopentenyl (C₅), cyclopentadienyl (C₅), cyclohexyl (C₆), cyclohexenyl (C₆), cycloheptyl (C₇), cyclooctanyl (C₈), decalinyl (C₁₀), 2,5-dimethylcyclopentyl (C₅), 3,5-dichlorocyclohexyl (C₆), 4-hydroxycyclohexyl (C₆), and 3,3,5-trimethylcyclohex-1-yl (C₆).
   ii) carbocyclic rings having two or more substituted or unsubstituted fused hydrocarbon rings, non-limiting examples of which include, octahydropentalenyl (C₈), octahydro-1H-indenyl (C₉), 3a,4,5,6,7,7a-hexahydro-3H-inden-4-yl (C₉), decahydroazulenyl (C₁₀)
   iii) carbocyclic rings which are substituted or unsubstituted bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.
2) The term “aryl” is defined herein as “units encompassing at least one phenyl or naphthyl ring and wherein there are no heteroaryl or heterocyclic rings fused to the phenyl or naphthyl ring and further each ring can be independently substituted with one or more moieties capable of replacing one or more hydrogen atoms.” The following are non-limiting examples of “substituted and unsubstituted aryl rings” which encompass the following categories of units:
   i) C₆ or C₁₀ substituted or unsubstituted aryl rings; phenyl and naphthyl rings whether substituted or unsubstituted, non-limiting examples of which include, phenyl (C₆), naphthylen-1-yl (C₁₀), naphthylen-2-yl(C₁₀), 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀).
   ii) C₆ or C₁₀ aryl rings fused with 1 or 2 saturated rings non-limiting examples of which include, bicyclo[4.2.0]octa-1,3,5-trienyl (C₈), and indanyl (C₉).
3) The terms “heterocyclic” and/or “heterocycle” are defined herein as “units comprising one or more rings having from 3 to 20 atoms wherein at least one atom in at least one ring is a heteroatom chosen from nitrogen (N), oxygen (O), or sulfur (S), or mixtures of N, O, and S, and wherein further the ring which comprises the heteroatom is also not an aromatic ring.” The following are non-limiting examples of “substituted and unsubstituted heterocyclic rings” which encompass the following categories of units:
   i) heterocyclic units having a single ring containing one or more heteroatoms, non-limiting examples of which include, diazirinyl (C₁), aziridinyl (C₂), urazolyl (C₂), azetidinyl (C₃), pyrazolidinyl (C₃), imidazolidinyl (C₃), oxazolidinyl (C₃), isoxazolinyl (C₃), isoxazolyl (C₃), thiazolidinyl (C₃), isothiazolyl (C₃), isothiazolinyl (C₃), oxathiazolidinonyl (C₃), oxazolidinonyl (C₃), hydantoinyl (C₃), tetrahydrofuranyl (C₄), pyrrolidinyl (C₄), morpholinyl (C₄), piperazinyl (C₄), piperidinyl (C₄), dihydropyranyl (C₅), tetrahydropyranyl (C₅), piperidin-2-onyl (valerolactam) (C₅), 2,3,4,5-tetrahydro-1H-azepinyl (C₆), 2,3-dihydro-1H-indole (C₈), and 1,2,3,4-tetrahydro-quinoline (C₉).
   ii) heterocyclic units having 2 or more rings one of which is a heterocyclic ring, non-limiting examples of which include hexahydro-1H-pyrrolizinyl (C₇), 3a,4,5,6,7,7a-hexahydro-1H-benzo[d]imidazolyl (C₇), 3a,4,5,6,7,7a-hexahydro-1H-indolyl (C₈), 1,2,3,4-tetrahydroquinolinyl (C₉), and decahydro-1H-cycloocta[b]pyrrolyl(C₁₀).
4) The term “heteroaryl” is defined herein as “encompassing one or more rings comprising from 5 to 20 atoms wherein at least one atom in at least one ring is a heteroatom chosen from nitrogen (N), oxygen (O), or sulfur (S), or mixtures of N, O, and S, and wherein further at least one of the rings which comprises a heteroatom is an aromatic ring.” The following are non-limiting examples of “substituted and unsubstituted heterocyclic rings” which encompass the following categories of units:
   i) heteroaryl rings containing a single ring, non-limiting examples of which include, 1,2,3,4-tetrazolyl (C₁), [1,2,3]triazolyl (C₂), [1,2,4]triazolyl (C₂), triazinyl (C₃), thiazolyl (C₃), 1H-imidazolyl (C₃), oxazolyl (C₃), furanyl (C₄), thiophenyl (C₄), pyrimidinyl (C₄), 2-phenylpyrimidinyl (C₄), pyridinyl (C₅), 3-methylpyridinyl (C₅), and 4-dimethylaminopyridinyl (C₅)
   ii) heteroaryl rings containing 2 or more fused rings one of which is a heteroaryl ring, non-limiting examples of which include: 7H-purinyl (C₅), 9H-purinyl (C₅), 6-amino-9H-purinyl (C₅), 5H-pyrrolo[3,2-d]pyrimidinyl (C₆), 7H-pyrrolo[2,3-d]pyrimidinyl (C₆), pyrido[2,3-d]pyrimidinyl (C₇), 2-phenylbenzo[d]thiazolyl (C₇), 1H-indolyl (C₈), 4,5,6,7-tetrahydro-1-H-indolyl (C₈), quinoxalinyl (C₈), 5-methylquinoxalinyl (C₈), quinazolinyl (C₈), quinolinyl (C₉), 8-hydroxy-quinolinyl (C₉), and isoquinolinyl (C₉).
5) C₁-C₆ tethered cyclic hydrocarbyl units (whether carbocyclic units, C₆ or C₁₀ aryl units, heterocyclic units, or heteroaryl units) which connected to another moiety, unit, or core of the molecule by way of a C₁-C₆ alkylene unit. Non-limiting examples of tethered cyclic hydrocarbyl units include benzyl C₁-C₆ having the formula:

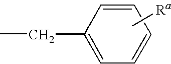

wherein R^{a} is optionally one or more independently chosen substitutions for hydrogen. Further examples include other aryl units, inter alia, (2-hydroxyphenyl)hexyl C₆-(C₆); naphthalen-2-ylmethyl C₁-C₁₀)_{,} 4-fluorobenzyl C₁-(C₆), 2-(3-hydroxy-phenyl)ethyl C₂-(C₆), as well as substituted and unsubstituted C₃-C₁₀ alkylenecarbocyclic units, for example, cyclopropylmethyl C₁-(C₃), cyclopentylethyl C₂-(C₅), cyclohexylmethyl C₁-(C₆). Included within this category are substituted and unsubstituted C₁-C₁₀ alkylene-heteroaryl units, for example a 2-picolyl C₁-(C₆) unit having the formula:

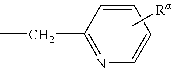

wherein R^{a} is the same as defined above. In addition, C₁-C₁₂ tethered cyclic hydrocarbyl units include C₁-C₁₀ alkyleneheterocyclic units and alkylene-heteroaryl units, non-limiting examples of which include, aziridinylmethyl C₁-(C₂) and oxazol-2-ylmethyl C₁-(C₃).

For the purposes of the present invention carbocyclic rings are from C₃ to C₂₀; aryl rings are C₆ or C₁₀; heterocyclic rings are from C₁ to C₉; and heteroaryl rings are from C₁ to C₉.

For the purposes of the present invention, and to provide consistency in defining the present invention, fused ring units, as well as spirocyclic rings, bicyclic rings and the like, which comprise a single heteroatom will be characterized and referred to herein as being encompassed by the cyclic family corresponding to the heteroatom containing ring, although the artisan may have alternative characterizations. For example, 1,2,3,4-tetrahydroquinoline having the formula:

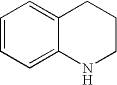

is, for the purposes of the present invention, considered a heterocyclic unit. 6,7-Dihydro-5H-cyclopentapyrimidine having the formula:

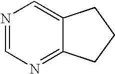

is, for the purposes of the present invention, considered a heteroaryl unit. When a fused ring unit contains heteroatoms in both a saturated ring (heterocyclic ring) and an aryl ring (heteroaryl ring), the aryl ring will predominate and determine the type of category to which the ring is assigned herein for the purposes of describing the invention. For example, 1,2,3,4-tetrahydro-[1,8]naphthyridine having the formula:

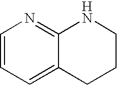

is, for the purposes of the present invention, considered a heteroaryl unit.

The term “substituted” is used throughout the specification. The term “substituted” is applied to the units described herein as “substituted unit or moiety is a hydrocarbyl unit or moiety, whether acyclic or cyclic, which has one or more hydrogen atoms replaced by a substituent or several substituents as defined herein below.” The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. The term substituted is used throughout the present specification to indicate that a hydrocarbyl moiety, inter alfa, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring (aryl ring)”, (N,N-dimethyl-5-amino)octanyl is a “substituted C₈ linear alkyl unit, 3-guanidinopropyl is a “substituted C₃ linear alkyl unit,” and 2-carboxypyridinyl is a “substituted heteroaryl unit.”

The following are non-limiting examples of units which can substitute for hydrogen atoms on a carbocyclic, aryl, heterocyclic, or heteroaryl unit:
i) C₁-C₄ linear or branched alkyl; for example, methyl (C₁), ethyl (C₂), n-propyl (C₃), iso-propyl (C₃), n-butyl (C₄), iso-butyl (C₄), sec-butyl (C₄), and tert-butyl (C₄);
ii) —OR³⁰; for example, —OH, —OCH₃, —OCH₂CH₃, —OCH₂CH₂CH₃;
iii) —C(O)R³⁰; for example, —COCH₃, —COCH₂CH₃, —COCH₂CH₂CH₃;
iv) —C(O)OR³⁰; for example, —CO₂CH₃, —CO₂CH₂CH₃, —CO₂CH₂CH₂CH₃;
v) —C(O)N(R³⁰)₂; for example, —CONH₂, —CONHCH₃, —CON(CH₃)₂;
vi) —N(R³⁰)₂; for example, —NH₂, —NHCH₃, —N(CH₃)₂, —NH(CH₂CH₃);
vii) halogen: —F, —Cl, —Br, and —I;
viii) —CHₘXₙ; wherein X is halogen, m is from 0 to 2, m+n=3; for example, —CH₂F, —CHF₂, —CF₃, —CCl₃, or —CBr₃; and
ix) —SO₂R³⁰; for example, —SO₂H; —SO₂CH₃; —SO₂C₆H₅
   wherein each R³⁰ is independently hydrogen, substituted or unsubstituted C₁-C₄ linear, branched, or cyclic alkyl; or two R³⁰ units can be taken together to form a ring comprising 3-7 atoms. Substituents suitable for replacement of a hydrogen atom are further defined herein below.

The compounds disclosed herein include all salt forms, for example, salts of both basic groups, inter alia, amines, as well as salts of acidic groups, inter alia, carboxylic acids. The following are non-limiting examples of anions that can form salts with basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form salts of acidic groups: sodium, lithium, potassium, calcium, magnesium, bismuth, and the like.

### Modes of Administration

In the methods of preventing or reducing loss of bone density, mass or strength, or of reducing osteoclast number or activity, the antiresorptive compound can be administered orally. Among the known antiresorptive compounds, alendronate, risedronate, ibandronate, tiludroante, etidronate and EVISTA® are recognized to be orally deliverable.

In the methods of preventing or reducing loss of bone density, mass or strength, or of reducing osteoclast number or activity, the antiresorptive compound can be administered intravenously. Among the known antiresorptive compounds, zoledronate, ibandronate, pamidronate and etidronate are recognized to the deliverable intravenously.

In the methods of preventing or reducing loss of bone density, mass or strength, or of reducing osteoclast number or activity, the antiresorptive compound can be administered subcutaneously. For example, this mode of administration is preferable for delivering Denosumab.

In the methods of preventing or reducing loss of bone density, mass or strength, or of reducing osteoclast number or activity, the antiresorptive compound can be administered intranasally. For example, this mode of administration is preferable for delivering calcitonin.

In the methods of preventing or reducing loss of bone density, mass or strength, or of reducing osteoclast number or activity, the antiresorptive compound can be administered by intramuscular injection, by intraperitoneal injection, transdermally, ophthalmically, vaginally, rectally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Bagshawe, 1989; Bagshawe et al., 1988; Battelli et al., 1992; Pietersz and McKenzie, 1992; Roffler et al., 1991; Senter et al., 1991; Senter et al., 1993). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., 1989; Litzinger and Huang, 1992). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, 1991).

### Time Course of Administration

In the methods of preventing or reducing loss of bone density, mass or strength, or of reducing osteoclast number or activity, the antiresorptive compound can be administered during the peri-radiation therapy period.

As used herein, the “peri-radiation period” is defined as a time period that is prior to the radiation exposure period or concurrent with at least a portion of the radiation exposure period. As used herein, the “radiation therapy period” refers to the time period for which radiation therapy is administered to a patient. This period will vary depending on the type of cancer, the condition of the subject, and other factors considered by the skilled person in designing and applying a radiation therapy regime. Numerous examples of radiation therapy regimes, and thus, peri-radiation periods, are described herein. For example, radiation therapy is usually administered from six to eight weeks for cervical cancer.

The peri-radiation period can last from about one (1) week to about the six (6) months. The period of antiresorptive treatment falls within the peri-radiation. The anti-resorptive therapy can begin one day to 4 months prior to beginning of the radiation therapy period and can continue up to 3 months after termination of radiation therapy. Thus, the antiresorptive therapy can last for about 4, 5, 6, 7, 8, 9, 10, 11, 12, 13, 14, 15, 16, 17, 18, 19, 20, 21, 22, 23, 24 or 25 weeks. The antiresorptive therapy can begin about 1, 2, 3, 4, 5 or 6 days or 1, 2, 3, 4, 5, 6, 7, 8, 9, 10, 11, 12, 13, 14, 15, 16, 17, 18, 19, 20, or 21 weeks prior to the radiation therapy period. The antiresorptive therapy can begin on the first day of radiation therapy and extend into the radiation therapy period, though it is likely to be less effective than if begun prior to the initiation of radiation therapy. The peri-radiation period can begin during the radiation therapy period or can begin up to about five (5) months prior to the initiation of radiation therapy and can extend through the full radiation therapy period. A subject who is about to receive radiation therapy is in the peri-radiation period, i.e., is within 5 months prior to the initiation of radiation therapy. In one example, the peri-radiation period includes time from before the initiation of radiation therapy and continuing past the completion of radiation therapy. In a more specific example, the peri-radiation therapy period can be a contiguous 6-month period including the entire period of radiation therapy. In one example, the peri-radiation therapy period includes a period of from 1 day to about four (4) months prior to the initiation of radiation therapy (exposure) and can extend past the end of the radiation therapy period. For example, if the antiresorptive therapy begins four (4) months before the radiation therapy period, and radiotherapy is two (2) months, the antiresorptive therapy ends when the radiation therapy ends. In a further example of the method, the peri-radiation-therapy period begins about three months prior to the initiation of radiation therapy, continues through the period of radiation therapy for about two months (about 8 weeks), and ends about one (1) month after the radiation therapy period ends. In a further example of the method, the peri-radiation-therapy period begins about two months prior to the initiation of radiation therapy, continues through the period of radiation therapy for about two months, and ends about two (2) months after the radiation therapy ends. Thus, in this example of the method, the peri-radiation-therapy period can begin about two months prior to the initiation of radiation therapy and ends six (6) months later. In a further example of the method, the peri-radiation-therapy period begins about one month prior to the initiation of radiation therapy, continues through the period of radiation therapy (typically from 4 to 8 weeks), and ends about 3 months after the radiation therapy period ends. In the case of some antiresorptive compounds, the antiresorptive therapy can be initiated as proximal as 2 weeks to the initiation of radiation therapy. For example, denosumab and zoledronate are potent enough that one injection about 1 week before radiation therapy is expected to be effective.

The time course of administration of antiresorptive compound to prevent or treat radiation-induced loss of bone density, mass or strength can range from 1, 2, 3, 4, 5, 6 months, with 4 to 6 months being a typical range. Also, if necessary due to repetition of the radiation therapy, the antiresorptive therapy of the present method can be repeated for the time periods indicated herein. The time course of administration of antiresorptive compound to prevent or treat radiation-induced loss of bone density, mass or strength can begin within from 1, 2, 3, 4, 5, 6 months of cancer diagnosis, with 4 to 6 months being a typical range.

Illustrative examples of time courses for administration of various doses for various antiresorptive compounds for use in the present methods are provided in Table 7. The information in this table includes known and tested time courses for various doses for various antiresorptive compounds. In addition to the information provided in the table, many scientific publications are available that provide dosing and time course information for specific antiresorptive compounds, and provide generally useful information for antiresorptive compound dosing and time course. These time courses are illustrative only, and not intended to limit the amount of time that a clinician might determine to administer the compound.

In addition to radiation therapy, there are other contexts in which exposure to radiation compromises bone integrity, for example, the exposure of astronauts to radiation during space travel. Lunar, Mars or asteroid rendezvous mission applicable doses for radiation is from 0.5-1.5 Gy and potentially 2 Gy if a large solar particle event occurs during a mission. Thus the method of preventing radiation-induced loss of bone density, strength or mass is applicable in any peri-radiation exposure context and duration.

### Dosing of Drugs

Provided herein is an amount of an antiresorptive compound effective to treat or prevent radiation induced loss of bone density, mass or strength.

As a general rule, the dose of antiresorptive compound that is administered for osteoporosis and higher should be effective. For example, amounts ranging from those for the treatment of osteoporosis/osteopenia to amounts for the treatment Paget's disease to amounts for the treatment of bone cancers (myeloma and metastases) and hypercalcemia from malignancy are expected to be effective. The following general calculations provide non-limiting guidance: osteoporosis X6=Paget's disease dose; and Paget's disease dose X3=bone cancer and hypercalcemia from malignancy dose. Again, as a non-limiting example, for treatment of radiation-induced bone loss, the lowest end of the range would be approximately the osteoporosis dose; and higher end of the range would be approximately the bone cancer dose. Osteoporosis relevant doses are readily available in the scientific literature. Similarly, Paget's disease therapy doses and bone cancer therapy doses are also described in the literature. Thus, the dose of a given antiresorptive compound for preventing or treating radiation-induced loss of bone density, mass or strength can include the dose of a given antiresorptive therapy for treatment of osteoporosis, or X2, X3, X4, X5 or X6 the dose for the same antiresorptive disclosed for the treatment of osteoporosis. In a further example, the dose of a given antiresorptive compound for preventing or treating radiation-induced loss of bone density, mass or strength can include an increase in the amount of the antiresorptive compound of 10%, 11%, 12%, 13%, 14%, 15%, 16%, 17%, 18%, 19%, 20%, 21%, 22%, 23%, 24%, 25%, 26%, 27%, 28%, 29%, 30% 31%, 32%, 33%, 34%, or 35% over the dose for the same antiresorptive compound disclosed for treating osteoporosis. Thus, for a subject who received antiresorptive therapy before a diagnosis of cancer, the amount of the dose of antiresorptive agent to treat the radiotherapy-induced bone loss in the present method is at least 25% greater than (to 30×=3000%) what that patient previously received before the cancer diagnosis (i.e., 25% greater than the dose being administered to treat the non-radiation-induced osteoporosis). For example, for a patient receiving risedronate for treatment of post-menopausal osteoporosis who is then diagnosed with cancer, the dose of residronate would be increased from 35 mg/week to 45 mg/week or more. Thus, the present method is different from treatment for non-radiation-induced osteoporosis, and can be used to treat a subject who has previously been treated for non-radiation-induced osteoporosis.

Furthermore, for a subject receiving antiresorptive therapy before diagnosis of cancer the frequency of administering an antiresorptive to treat radiotherapy induced bone loss is 25% more often than what the patient was previously scheduled to receive. For example, for a patient receiving injections of denosumab for treatment of post-menopausal osteoporosis who is then diagnosed with cancer, the frequency of injection of six months between injections is increased to a frequency of 4.5 months between injections, or more frequently. As an additional example, for a patient receiving injections of zoledronate for treatment of post-menopausal osteoporosis, the frequency of twelve months between injections is increased to 9 months between injections, or more frequently. Thus, in the present method, the total amount (a combination of amount and frequency) of an antiresorptive agent administered is at least 25% more than the total amount of the same antiresporptive agent adminstered for the treatment of non-radiation-induced osteoporosis.

The total dose used in the treatment example (˜0.1 mg/kg/week risedronate subcutaneously administered) has been shown to improve bone volume and trabecular number in rats one month after causing glucocorticoid-induced osteoporosis (GIO). This total dose has thus been effective in preserving and improving bone microarchitecture in animal models of diseases that cause bone loss. In one study of GIO, 0.1 mg/kg/week of risedronate administered subcutaneously increased bone volume and fraction by reducing turnover (Iwamoto et al., 2006).

Estrogen deficiency-induced osteoporosis can be accomplished in animal models by removing the ovaries (OVX), which primarily causes bone loss by increasing turnover. From OVX studies, 0.5 mg/kg/day is effective when administered subcutaneously. This would equate to 3.5 mg/kg/week. It is reasonable to assume a 1% oral bioavailability (realistically it is nearer the order of 0.5-0.7%) when converting to subcutaneous doses. This would equate to 0.005 mg/kg/day, or approximately 35 mg/kg/wk. However, a major concern is not only preventing bone loss, but preserving strength (preventing fractures is the ultimate goal). In one study comparing bones of aged rats (9 months then allowed to mature 60 days), there were no histomorphometric changes in the tibial diaphysis of rats that had received 0.02 or 0.04 mg/kg/wk of risedronate (Ma et al., 1997). OVX rat-studies of risedronate administered orally revealed that improvement in bone mineral density and preservation of microarchitecture generally occurred in doses greater than 0.5 mg/kg/day [other doses used were 0.1 mg/kg/day (Erben et al., 2002) and 2.5 mg/kg/day (Otomo et al., 2004)].

Doses of risedronate that are effective in preserving microarchitecture include 0.5 and 2.5 mg/kg/day oral administration (Borah et al., 2002; Otomo et al., 2004). However, the results in preserving architecture, which is integral to preserving strength or bone, was more pronounced in animal models (minipigs) using 2.5 mg/kg/day than 0.5 mg/kg/day (Borah et al., 2002). Assuming a 1% oral bioavailability, a 2.5 mg/kg/day oral=25 μg/kg/day subcutaneous. If the bioavailability was 0.5%, this would be 12.5 μg/kg/day. The 15 μg/kg/dose is a compromise between the two. This is approximately 0.1 mg/kg/week, which was used in the treatment examples.

As one of skill in the art would understand, the exact dose of the antiresorptive compound described herein can be determined based on the drug, the subject, and the time course of radiation exposure (radiotherapy period). Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

Illustrative examples of doses for various antiresorptive compounds for use in the present methods are provided in Table 7. The information in this table includes information for known and tested doses for various antiresorptive compounds. Also provided are the in vivo data of the examples, which are extrapolatable to human doses when viewed in conjunction with known and tested doses for the same compound. In addition to the information provided in the table, many scientific publications are available that provide dosing information for the specific antiresorptive compounds and generally useful information for antiresorptive dosing. The ranges disclosed in the table are considered to disclose each and every dose between the upper and lower ends of the range. Also, where a dose is given in mg/kg, that dose can be routinely converted to a specific mass/amount. Likewise, a mg/kg amount for a subject can be routinely determined from an absolute amount based on the subject's weight.

Although the effective dose is dependent on the specific antiresorptive compound, an oral dose will typically be higher than an intravenous dose for a given compound. For example, if the antiresorptive compound is to be delivered orally, an effective amount would be at least 20 micrograms/kg/day, 140 micrograms/kg/week, or 6 mg/kg/month or 2.5 mg/day, 15 mg/week or 75 mg/month orally and could range at least as high as 50 mg/kg/day, or 350 mg/kg/week, or 1.5 grams/kg/month or 1 gram/day, or 7 grams/week or 30 grams per month.

In the methods of preventing or reducing radiation-associated loss of bone density, mass or strength or of reducing osteoclast number or activity, by intravenous administration, an amount of antiresorptive compound effective to prevent radiation-induced bone loss would be at least a single 8 micrograms/kg injection or 1 mg injection. Although it is expected that the daily intravenous dose of such a compound would be significantly less than 8 micrograms/kg, for example, 1 microgram/kg/day or less, it is not typical to administer drugs intravenously on a daily basis if weekly or longer periods between doses can be accommodated. This dose could range as high as 0.25 mg/kg/week, or 1 mg/kg/month, or 5 mg/week or 20 mg/month.

In the methods of preventing or reducing radiation-associated loss of bone density, mass or strength, or of reducing osteoclast number or activity, by intranasal administration, e.g., by nasal spray or aerosol, an amount of antiresorptive compound effective to prevent radiation-induced bone loss would be at least about 100 IU daily and up to 400 IU daily.

In the methods of preventing or reducing radiation-associated loss of bone density, mass or strength, or of reducing osteoclast number or activity, by subcutaneous, intramuscular or other injection administration, an amount of antiresorptive compound effective to prevent or reduce radiation-induced bone loss would range from at least about a single 10 micrograms/kg to about a single 200 microgram dose. An effective dose could range as high as 10 mg/kg/week, or 40 mg/kg/month, or 120 mg/kg/12-weeks, or 200 mg/week, or 800 mg/month, or 2.5 grams/12-weeks.

In the method of preventing or reducing radiation-associated loss of bone density, mass or strength, or of reducing osteoclast number or activity, the amount of antiresorptive compound effective to prevent radiation induced bone loss is at least 20 micrograms/kg/day orally. In the method of preventing or reducing radiation-associated loss of bone density, mass or strength the amount of antiresorptive compound effective to prevent radiation induced bone loss in a patient that has not previously received an antiresorptive agent is at least 20 micrograms/kg/day orally.

In the method of preventing or reducing radiation-associated loss of bone density, mass or strength, or of reducing osteoclast number or activity, the amount of antiresorptive compound effective to prevent radiation induced bone loss is at least a single administration of 10 micrograms/kg subcutaneously, intramuscularly, or by other injection method. In the method of preventing or reducing radiation-associated loss of bone density, mass or strength the amount of antiresorptive compound effective to prevent radiation induced bone loss in a patient that has not previously received an antiresorptive agent is at least a single administration of 10 micrograms/kg subcutaneously, intramuscularly, or by other injection method.

In the method of preventing or reducing radiation-associated loss of bone density, mass or strength, or of reducing osteoclast number or activity, the amount of antiresorptive compound effective to prevent radiation induced bone loss is at least a single administration of 8 micrograms/kg intravenously. In the method of preventing or reducing radiation-associated loss of bone density, mass or strength the amount of antiresorptive compound effective to prevent radiation induced bone loss in a patient that has not previously received an antiresorptive agent is at least a single administration of 8 micrograms/kg intravenously

### Combination Therapy

The antiresorptive therapy of the present method can be combined with antioxidant therapy. Also, since certain antioxidants have been identified as beneficial in the prevention and/or treatment of loss of bone mass, density or strength, it is recognized that antioxidants capable of treating or preventing loss of bone mass, density or strength can be used independently in the present methods.

The antiresorptive therapy of the present method can be combined with vitamin D and its derivatives ((5Z,7E)-(3S)-9,10-seco-5,7,10(19)-cholestatrien-3-ol, Calcidiol, Calcitriol, ercalciol or ergocalciferol, Ercalcitriol, (24S)-methylcalciol or 22,23-dihydroercalciol, Calcitetrol, (6Z)-tacalciol, Tacalciol, (5E)-isocalciol, and Dihydroercalciol), or vitamin K and its derivatives, or calcium and its derivatives (including calcium citrate, calcium carbonate, tribasic calcium phosphate, calcium lactate, calcium gluconate, bone meal, or calcium magnesium carboneat [dolomite]) as well as with other compounds that treat or prevent bone diseases.

The antiresorptive therapy of the present method can be combined with anti-inflammatory compounds (for example, cox-1 and cox-2 inhibitors, IL-1 inhibiting compounds such as anakinra or TNFα inhibiting compounds such as etanercept, infliximab or adalimumab). Also, any anti-inflammatory compounds that are shown to have a beneficial effect in treating or preventing loss of bone mass, density or strength can be used independently in the present methods.

The antiresorptive therapy of the present method can be combined with any known anti-cancer therapy. Thus, the antiresorptive can be combined with an anti-angiogenic compound (e.g., endostatin, thrombospondin, EMAP-II, IP-10, angiostatin, vasostatin, vasculostatin, IL-12, platelet factor 4, cleavage products of collagen VIII, cleavage products of collagen XV, restatin) or other anti-cancer compound whether now known or later developed. The combination of an antiresorptive compound and an anti-cancer compound can be in a pharmaceutical formulation that comprises a mixture of both compounds. The combination can constitute concurrent administration of the antiresorptive compound and the anti-cancer compound as separate pharmaceutical formulations.

Numerous anti-cancer drugs are available for combination with the present method and compositions. The following are lists of anti-cancer (anti-neoplastic) drugs that can be used in conjunction with the presently disclosed antiresorptive compounds.

Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The herein provided composition can further comprise one or more additional radiosensitizers. Examples of known radiosensitizers include gemcitabine, 5-fluorouracil, pentoxifylline, and vinorelbine (Anderson et al., 2008; Lawrence et al., 2001; Morgan et al., 2008).

The majority of chemotherapeutic drugs can be divided in to: alkylating agents, antimetabolites, anthracyclines, plant alkaloids, topoisomerase inhibitors, monoclonal antibodies, and other antitumour agents. All of these drugs affect cell division or DNA synthesis. Some newer agents don't directly interfere with DNA. These include the new tyrosine kinase inhibitor imatinib mesylate (Gleevec® or Glivec®), which directly targets a molecular abnormality in certain types of cancer (chronic myelogenous leukemia, gastrointestinal stromal tumors). In addition, some drugs can be used which modulate tumor cell behaviour without directly attacking those cells. Hormone treatments fall into this category of adjuvant therapies.

The chemotherapeutic of the disclosed method can be an alkylating agent. Alkylating agents are so named because of their ability to add alkyl groups to many electronegative groups under conditions present in cells. Cisplatin and carboplatin, as well as oxaliplatin are alkylating agents. Other agents are mechloethamine, cyclophosphamide, chlorambucil. They work by chemically modifying a cell's DNA.

The chemotherapeutic of the disclosed method can be an anti-metabolite. Anti-metabolites masquerade as purine ((azathioprine, mercaptopurine)) or pyrimidine—which become the building blocks of DNA. They prevent these substances becoming incorporated in to DNA during the ‘5’ phase (of the cell cycle), stopping normal development and division. They also affect RNA synthesis. Due to their efficiency, these drugs are the most widely used cytostatics.

The chemotherapeutic of the disclosed method can be a plant alkaloids or terpenoids. These alkaloids are derived from plants and block cell division by preventing microtubule function. Microtubules are vital for cell division and without them it can not occur. The main examples are vinca alkaloids and taxanes.

The chemotherapeutic of the disclosed method can be a vinca alkaloid. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine, Vinblastine, Vinorelbine, Vindesine, and Podophyllotoxin. Podophyllotoxin is a plant-derived compound used to produce two other cytostatic drugs, etoposide and teniposide. They prevent the cell from entering the G1 phase (the start of DNA replication) and the replication of DNA (the S phase). The exact mechanism of its action still has to be elucidated. The substance has been primarily obtained from the American Mayapple (Podophyllum peltatum). Recently it has been discovered that a rare Himalayan Mayapple (Podophyllum hexandrum) contains it in a much greater quantity, but as the plant is endangered, its supply is limited. Studies have been conducted to isolate the genes involved in the substance's production, so that it could be obtained recombinantively.

The chemotherapeutic of the disclosed method can be a taxane. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

The chemotherapeutic of the disclosed method can be a topoisomerase inhibitor. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include the camptothecins irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

The chemotherapeutic of the disclosed method can be an antitumour antibiotic (Antineoplastics). The most important immunosuppressant from this group is dactinomycin, which is used in kidney transplantations.

The chemotherapeutic of the disclosed method can be an (monoclonal) antibody. Monoclonal antibodies work by targeting tumour specific antigens, thus enhancing The host's immune response to tumour cells to which the agent attaches itself. Examples are trastuzumab (Herceptin), cetuximab, and rituximab (Rituxan or Mabthera). Bevacizumab is a monoclonal antibody that does not directly attack tumor cells but instead blocks the formation of new tumor vessels.

The chemotherapeutic of the disclosed method can be a hormonal therapy. Several malignancies respond to hormonal therapy. Strictly speaking, this is not chemotherapy. Cancer arising from certain tissues, including the mammary and prostate glands, may be inhibited or stimulated by appropriate changes in hormone balance. Steroids (often dexamethasone) can inhibit tumour growth or the associated edema (tissue swelling), and may cause regression of lymph node malignancies. Prostate cancer is often sensitive to finasteride, an agent that blocks the peripheral conversion of testosterone to dihydrotestosterone. Additionally, prostate cancer is often treated with androgen blocking or antiandrogen compounds generally referred to as androgen deprivation therapy; these agents include flutamide, bicalutamide, nilutamide, cyproterone acetate. A surgical method of blocking androgen deprivation is orchiectomy. Adrenal androgens, such as, can also be blocked in prostate cancer patients with ketoconazole and aminoglutethimide. Gonadotropin-releasing hormone analogue, also known as a GnRH analogue, or GnRH antoginists (e.g., abrelix) or GnRH agonists such as leuprolide, goserelin, triptorelin, buserelin, abiraterone acetate result in lower LH and thus can inhibit tumor growth. Breast cancer cells often highly express the estrogen and/or progesterone receptor. Inhibiting the production (with aromatase inhibitors) or action (with tamoxifen) of these hormones can often be used as an adjunct to therapy. Gonadotropin-releasing hormone agonists (GnRH), such as goserelin possess a paradoxic negative feedback effect followed by inhibition of the release of FSH (follicle-stimulating hormone) and LH (luteinizing hormone), when given continuously. Some other tumours are also hormone dependent, although the specific mechanism is still unclear.

### Pharmaceutical Compositions

Provided herein are compositions comprising antiresorptive compounds in combination with a pharmaceutically acceptable carrier. Also, provided are compositions comprising antiresorptive compounds and anti-cancer drugs (e.g., chemotherapy) and or radiation-enhancing drugs in a pharmaceutically acceptable carrier.

Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antiresorptive compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Formulations for topical administration can comprise as a vehicle lower aliphatic alcohols, polyglycols, esters of fatty acids, oils, fats and or silicones. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Identification of Target Patients

The present methods are applicable in types of cancers where normal bone is likely to receive doses of radiation, including colon, rectal, anal, cervical, uterus, ovary, urinary/bladder, prostate, stomach, esophagus, lung, brachial, and breast tumors. As used herein, “normal bone” is bone that is not cancerous in any way, i.e., affected by neither primary bone cancer nor metastatic bone cancer. Additionally, patients requiring bone marrow transplantation (e.g. for leukemia and lymphatic cancers) receive whole body irradiation, and can be subject to the present methods.

The present methods are applicable to a subject who has or is about to receive cumulative doses of radiation administered to the tumor or whole body as low as 1 Gy and as high as, but not excluding higher than, 100Gy. Thus, the present methods are applicable to a subject who has or is about to receive cumulative doses of radiation administered to the tumor or whole body of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 95, 96, 97, 98, 99, 100 Gy or higher.

In specific embodiments of the present invention, the bone exposed to ionizing is normal, non-cancerous, bone not directly in contact with a tumor. In specific embodiments of the present methods, the subject does not have a primary bone cancer, such as osteosarcoma. In specific embodiments of the present methods, the subject does not have a primary bone cancer, such as chondrosarcoma. In specific embodiments of the present methods, the subject does not have a primary bone cancer, such as multiple myeloma. In specific embodiments of the present methods, the subject does not have a metastatic cancer (i.e., a cancer that has spread from a primary tumor). In specific embodiments of the present methods, the subject does not have a metastatic bone cancer (i.e., a cancer that has spread to bone from a primary tumor elsewhere).

In specific embodiments of the present methods, the subject does not have leukemia or lymphoma. In specific embodiments of the present methods, the subject does not have uterine cancer. In specific embodiments of the present methods, the subject does not have cervical cancer. In specific embodiments of the present methods, the subject does not have breast cancer. In specific embodiments of the present methods, the subject does not have colon cancer. In specific embodiments of the present methods, the subject does not have ovarian cancer. In specific embodiments of the present methods, the subject does not have head or neck cancer. In specific embodiments of the present methods, the subject does not have avascular necrosis, osteonecrosis or osteoradionecrosis. In specific embodiments of the present methods, the subject does not have peritoneal cancer. In specific embodiments of the present methods, the subject does not have thoracic cancer.

In specific embodiments of the present methods, the subject is not receiving non-ionizing radiation. Thus, in this embodiment, the patient is not receiving ultraviolet radiation, visible light, near infrared radiation, far infrared radiation, microwaves or radioraves to improve wound healing or for any other reason.

In specific embodiments of the present methods, the subject has not received whole body irradiation. In specific embodiments of the present methods, the subject is not a child (pediatric patient). Thus, the subject is an adult. In specific embodiments of the present methods, the subject has not been administered radiotherapy for palliative reasons (e.g., for control of pain). In specific embodiments of the present methods, the subject does not have terminal cancer (a stage of cancer where remission is unlikely). In specific embodiments of the present methods, the subject has not received internal radiation (Brachytherapy). In specific embodiments of the present methods, the antiresorptive treatment is not given to prevent bone loss that is predominantly caused by chemotherapy or glucocorticoid therapy.

In specific embodiments of the present methods, the subject has not received only a cumulative dose of radiation to any part of the body lower than 1.0 Gy. For example, in specific embodiments of the present methods, the subject has not been exposed only to diagnostic x-rays, e.g., CT-scan. Cumulative dose excludes “background” exposure or radiation that is environmental in nature (from natural geological sources).

In specific embodiments of the present methods, the subject has not previously been treated with an antiresorptive agent. In specific embodiments of the present methods, the subject has not previously been treated with an antiresorptive agent at a dose that exceeds by at least 25% the standard dose or dose frequency for the treatment of osteoporosis (e.g., see the amounts listed in Table 7 for osteoporosis treatment). In specific embodiments of the present methods, the subject has not previously been treated with an antiresorptive agent at a dose or dose frequency that exceeds the standard dose for treatment of osteoporosis by at least 25% at the time cancer is discovered or diagnosed. In specific embodiments of the present methods, the subject has not already been treated with an antiresorptive agent at a dose that exceeds the standard dose or dose frequency for treatment of osteoporosis by at least 25% within 1, 2, 3, 4, 5 or 6 months prior or during radiation therapy. In specific embodiments of the present methods, the subject has not been administered an antiresorptive agent for the treatment of a skeletal disorder. In specific embodiments of the present methods, the subject has not been administered an antiresorptive agent for the treatment of a skeletal disorder at a dose that exceeds the standard dose or dose frequency for treatment of osteoporosis by at least 25%. In specific embodiments of the present methods, the subject has not been administered an antiresorptive agent for the prevention or treatment of bone loss resulting only from chemotherapy. In specific embodiments of the present methods, the subject has not been administered an antiresorptive agent for the prevention or treatment of bone loss resulting from chemotherapy at a dose that exceeds the standard dose or dose frequency for treatment of osteoporosis by at least 25%. In specific embodiments of the present methods, the subject has not been administered an antiresorptive agent for the treatment of osteopenia. In specific embodiments of the present methods, the subject has not been administered an antiresorptive agent for the treatment of osteopenia at a dose that exceeds the standard dose or dose frequency for treatment of osteoporosis by at least 25%. In specific embodiments of the present methods, the subject has not been administered an antiresorptive agent for the treatment of osteomalacia. In specific embodiments of the present methods, the subject has not been administered an antiresorptive agent for the treatment of osteogenesis imperfecta. In specific embodiments of the present methods, the subject has not been administered an antiresorptive agent for the treatment for hypercalcaemia. In specific embodiments of the present methods, the subject has not been administered an antiresorptive agent for the treatment of fracture wherein the anti-resporptive is administered to improve fracture healing, particularly when orthopaedic intervention (plates, rod, nails, screws, wires) are used. In specific embodiments of the present methods, the subject has not been administered an antiresorptive agent for the treatment of heterotopic ossification (e.g., bone forming within muscle) and/or ectopic bone formation (general bone forming where it should not). In specific embodiments of the present methods, the subject has not been administered an antiresorptive agent for the treatment of rickets disease. In specific embodiments of the present methods, the subject has not been administered an antiresorptive agent for the treatment of Paget's disease. In specific embodiments of the present methods, the subject has not been administered an antiresorptive agent for the treatment of osteoarthritis at a dose that exceeds the standard dose or dose frequency for treatment of osteoporosis by at least 25%. In specific embodiments of the present methods, the subject has not been administered an antiresorptive agent for the treatment of rheumatoid arthritis at a dose that exceeds the standard dose or dose frequency for treatment of osteoporosis by at least 25%. In specific embodiments of the present methods, the subject has not been administered an antiresorptive agent for the treatment of parathyroid or thyroid disorders, e.g, Graves disease, or thyroid cancer, at a dose that exceeds the standard dose or dose frequency for treatment of osteoporosis by at least 25%.

In specific embodiments of the present methods, the subject has not previously been administered an antiresorptive compound to prevent or treat steroid (e.g., glucocorticoid)-induced osteoporosis only. In specific embodiments of the present methods, the subject has not previously been administered an antiresorptive compound to prevent or treat steroid (e.g., glucocorticoid)-induced osteoporosis, at a dose that exceeds the standard dose or dose frequency for treatment of osteoporosis by at least 25%. In specific embodiments of the present methods, the subject has not previously been administered an antiresorptive agent to prevent or treat bone loss resulting from androgen or estrogen blocking compounds only. In specific embodiments of the present methods, the subject has not previously been administered an antiresorptive agent to prevent or treat bone loss from androgen or estrogen blocking compounds, at a dose that exceeds the standard dose or dose frequency for treatment of osteoporosis by at least 25%.

In specific embodiments of the present methods, the subject has not been administered a bisphosphonate for the treatment of cancer for curative purposes. In specific embodiments of the present methods, the subject has not been administered a bisphosphonate to improve the efficacy of radiotherapy for curative purposes.

In specific embodiments of the present methods, the patient has previously received radiation therapy to treat cancer. In specific embodiments of the present methods, the subject has previously received radiation therapy, but was not treated with an antiresorptive agent prior to or during radiation therapy. In specific embodiments of the present methods, the subject has previously received radiation therapy, but was not treated prior to or during the previous radiation therapy, with an antiresorptive agent at a dose or dose frequency that exceeds the standard dose for treatment of osteoporosis by at least 25%.

In specific embodiments of the present methods, the subject has not previously been administered alendronate at any time within 1, 2, 3, 4, 5, or 6 months prior to initiation of or within 1, 2, 3, 4, 5, or 6 months of completion of previously administered radiation therapy. In specific embodiments of the present methods, the subject has not previously been administered alendronate at any time within 1, 2, 3, 4, 5, or 6 months prior to initiation of or within 1, 2, 3, 4, 5, or 6 months of completion of previously administered radiation therapy at a dose that exceeds by at least 25% the standard dose or dose frequency of alendronate for treatment of osteoporosis. In specific embodiments of the present methods, the subject has not previously been administered risedronate within 1, 2, 3, 4, 5, or 6 months prior to initiation of or after completion of radiation therapy at a dose that exceeds the standard dose or dose frequency of risedronate for treatment of osteoporosis by at least 25%. In specific embodiments of the present methods, the subject has not previously been administered ibandronate within 1, 2, 3, 4, 5, or 6 months prior to initiation of or after completion of radiation therapy at a dose that exceeds the standard dose or dose frequency of ibandronate for treatment of osteoporosis by at least 25%. In specific embodiments of the present methods, the subject has not previously been administered zoledronate within 1, 2, 3, 4, 5, or 6 months prior to initiation of or after completion of radiation therapy at a dose that exceeds the standard dose or dose frequency of zoledronate for treatment of osteoporosis by at least 25%. In specific embodiments of the present methods, the subject has not previously been administered pamidronate within 1, 2, 3, 4, 5, or 6 months prior to initiation of or after completion of radiation therapy at a dose that exceeds the standard dose or dose frequency of pamidronate for treatment of osteoporosis by at least 25%. In specific embodiments of the present methods, the subject has not previously been administered etidronate within 1, 2, 3, 4, 5, or 6 months prior to initiation of or after completion of radiation therapy at a dose that exceeds the standard dose or dose frequency of etidronate for treatment of osteoporosis by at least 25%. In specific embodiments of the present methods, the subject has not previously been administered tiludronate within 1, 2, 3, 4, 5, or 6 months prior to initiation of or after completion of radiation therapy at a dose that exceeds the standard dose or dose frequency of tiludronate for treatment of osteoporosis by at least 25%. In specific embodiments of the present methods, the subject has not previously been administered EVISTA® within 1, 2, 3, 4, 5, or 6 months prior to initiation of or after completion of radiation therapy at a dose that exceeds the standard dose or dose frequency of EVISTA® for treatment of osteoporosis by at least 25%. In specific embodiments of the present methods, the subject has not previously been administered denosumab within 1, 2, 3, 4, 5, or 6 months prior to initiation of or after completion of radiation therapy at a dose that exceeds the standard dose or dose frequency of denosumab for treatment of osteoporosis by at least 25%. In specific embodiments of the present methods, the subject has not previously been administered calcitonin within 1, 2, 3, 4, 5, or 6 months prior to initiation of or after completion of radiation therapy at a dose that exceeds the standard dose or dose frequency of calcitonin for treatment of osteoporosis by at least 25%.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

These examples, as a whole, demonstrate an entirely novel claim, not previously described in the literature, that administration of antiresorptive compounds to a patients receiving radiotherapy, or being exposed to ionizing radiation, will have the important health benefit of preventing the radiation-induced activation of osteoclasts that results in loss of bone mass and strength. The experiments that resulted in the collection of data presented in these Examples have never been previously performed.

### Example 1

### Radiation Exposure Causes an Increase in Osteoclast Number and Activity in Mice

### Methods

Mice were exposed to a whole body 2 Gy dose of x-rays. Serum was collected from each mouse one and three days after exposure to identify changes in circulating markers of bone resorption by osteoclasts (TRAP5b; tartrate resistant acid phosphatase, type 5 is osteoclast specific) or formation by osteoblasts (osteocalcin). Additionally, bone marrow was flushed from the femurs of these animals, and cultured for either one week (cultured on chamber slides and stained with TRAP to identify changes in osteoclast number after irradiation) or two weeks (cultured on hydroxyapatite disks to identify changes in bone resorption from irradiated marrow).

Three days after irradiation, histology was performed on another set of mice. Histology of the tibiae was also analyzed in order to determine the percentage of bone covered by osteoclasts and osteoblasts at this early (3-day) time point.

### Results

### Example 1a

### Serum Markers for Osteoclast and Osteoblast Activity

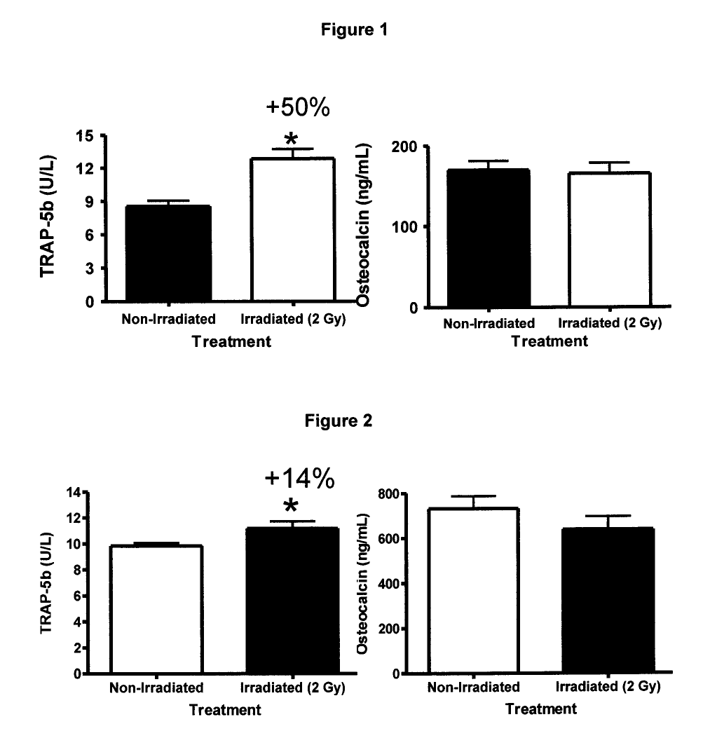

Radiation increased levels of TRAP5b both one (FIG. 1) and three (FIG. 2) days after exposure demonstrating an activation of osteoclasts at these time points. There was no change in osteocalcin levels at either time point (FIGS. 1 and 2). This activation of osteoclasts with no change in osteoblast activity contradicts the preponderance of the published literature that states that radiation primarily inhibits osteoblast function.

### Example 1b

### Osteoclast Number from Bone Marrow Culture after Irradiation

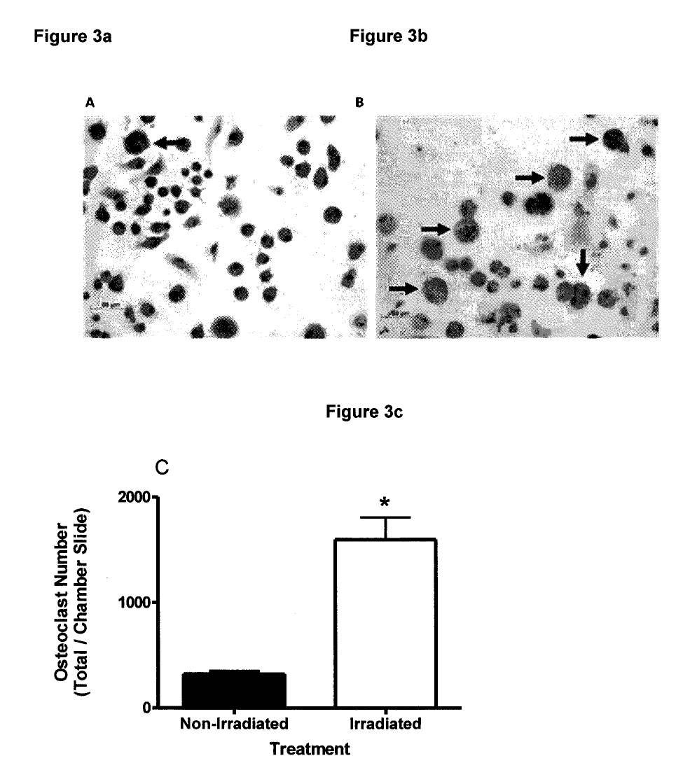

After one week of culture, the number of TRAP positive, multinucleated osteoclasts was greater from irradiated marrow harvested one day after exposure then than from control animals (Table 1). A mean increase of approximately 395% was observed from irradiated marrow relative to non-irradiated marrow (P<0.05; FIG. 3).

### Example 1c

### Osteoclast Resorption of Mineral from Bone Marrow Culture after Irradiation

The area of mineral resorbed on the hydroxyapatite disks (the mineral component of bone) after culture with irradiated marrow for 2 weeks was significantly greater than when cultured with non-irradiated marrow (Table 1). Total resorbed area was approximately 390% greater after irradiation (P<0.05).

Collectively, these culture data indicate that from in vitro studies, radiation profoundly increases osteoclast number and resorption capacity from bone marrow collected from irradiated mice.

TABLE 1
Quantification of osteoclast numbers and hydroxyapatite
resorption from bone marrow culture.

| | Mean Control | Mean 2 Gy | P Value |
|---|---|---|---|
| Osteoclast number | 322 ± 30 | 1600 ± 207 | 0.018 |
| Resorption pit number | 294 ± 86 | 350 ± 156 | 0.080 |
| Total resorption pit area (mm) | 0.101 ± 0.027 | 0.413 ± 0.184 | 0.043 |
| Average resorption pit area | 22400 ± 5000 | 37700 ± 16900 | 0.007 |
| (μm) | | | |

Notes:
A repeated measures ANOVA was used to compare Between Group effects (control vs. 2 Gy irradiated) by analyzing data from each replicate for each of three samples within each group.

### Example 1d

### Histology Analysis Osteoclast, Osteoblast and Eroded Surface

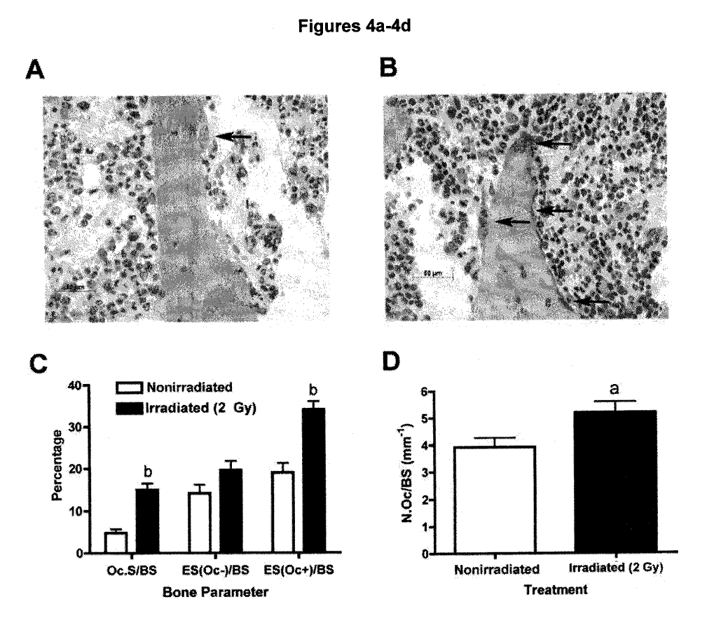

The present experiment showed no difference in total bone surface, OB surface (indicating no effect on osteoblasts at this early time after exposure), or erosion surface (P>0.05; FIG. 4 and Table 2). OC surface was 4.8% in the control group and ˜15% in the irradiated group. Thus, there was a 210% increase in OC surface (P<0.001).

Examination of functional bone loss by micro-computed tomography (microCT: Table 2) indicates no changes at this stage (BV/TV, Conn.den, Tb.Th, Tb.N, Tb.Sp). This is consistent with the non-significant differences in eroded surface: osteoclast activation/number proceeds eroded bone surface which proceeds functional changes as measured by microCT.

TABLE 2
Histological, microCT and serum marker parameters
from mice 3 days after radiation exposure.
Histological Parameters, Trabecular
Microarchitectural Parameters, and Bone
Metabolism Markers Collected from Mice Three
Days after Whole-Body Irradiation with
2 Gy of X Rays

| | | |
|---|---|---|
| | Irradiated | Percentage |
| Nonirradiated | (2 Gy) | change |

Histological parameters (n = 6/group)

| | | | |
|---|---|---|---|
| BS (mm) | 6.26 ± 0.57 | 5.69 ± 0.33 | −9% |
| ES(Oc+)/BS (%) | 19.0 ± 2.3 | 34.1 ± 1.9^{a} | +79%^{a} |
| ES(Oc−)/BS (%) | 14.2 ± 2.0 | 19.6 ± 2.2 | +38% |
| Oc.S/BS (%) | 4.8 ± 0.9 | 15.0 ± 1.5^{a} | +213%^{a} |
| Ob.S/BS (%) | 14.6 ± 2.2 | 14.2 ± 1.9 | −3% |
| N.Oc/BS (mm⁻¹) | 4.5 ± 0.4 | 6.5 ± 0.5^{b} | +44%^{b} |

Trabecular microarchitecture (n = 10/group)

| | | | |
|---|---|---|---|
| BV/TV (%) | 6.76 ± 0.47 | 7.45 ± 0.10 | +10% |
| Conn.D (N/mm³) | 27 ± 5 | 35 ± 4 | +30% |
| Tb.Th (μm) | 43.7 ± 0.5 | 43.2 ± 0.5 | −1% |
| Tb.N (N/mm³) | 3.37 ± 0.12 | 3.56 ± 0.08 | +6% |
| Tb.Sp (μm) | 297 ± 12 | 280 ± 7 | −6% |

Serum bone metabolism markers (n = 8/group)

| | | | |
|---|---|---|---|
| TRAP-5b (U/liter) | 9.84 ± 0.24 | 11.17 ± 0.55^{b} | +14%^{b} |
| Osteocalcin (ng/ml) | 732 ± 55 | 638 ± 58 | −13% |

Notes.
BV/TV, bone volume fraction, connectivity density (Conn.D);
trabecular thickness (Tb.Th.);
trabecular number (Tb.N); trabecular separation (Tb.Sp.);
bone surface (BS);
eroded surface with the inclusion of osteoclast surface [ES(Oc+)/BS];
eroded surface with the exclusion of osteoclast surface [ES(Oc−)/BS];
osteoclast surface (Oc.S);
osteoblast surface (Ob.S);
number of osteoclasts (N.Oc/BS).
All values are mean ± SE.
^{a}P < 0.001 and
^{b}P < 0.05 after t test.

### Example 2

### Radiation Therapy Causes a Rapid Decline in Bone Mass in Cervical Cancer Patients

Ongoing Clinical Trial Identifies Radiation-Induced Declines in vBMD and Strength

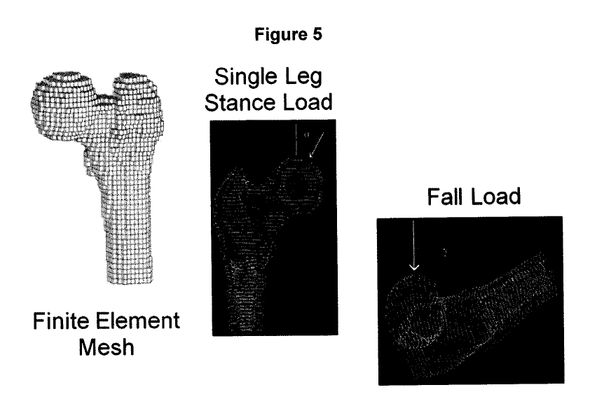

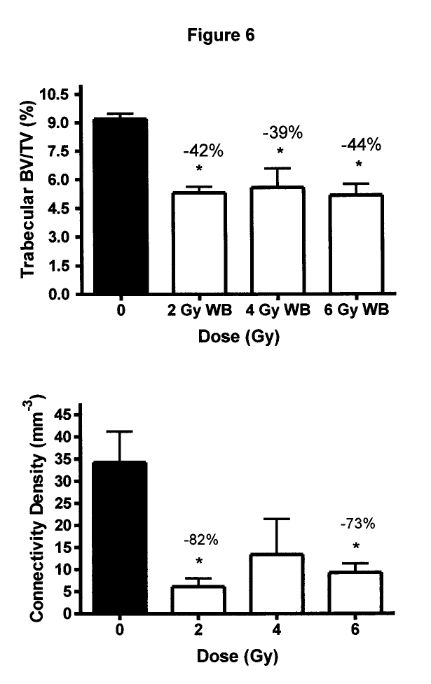

A clinical trial is in progress to examine the loss of bone density after radiotherapy in cervical and prostate cancer patients. Eight weeks after exposure, the first patient exhibited a 7% and 45% decline in vBMD at the femoral neck and greater trochanter as measured by quantitative computed tomography (QCT) compared to pre-treatment QCT scans, respectively. Finite element modeling of these data (FIG. 5) indicates a 16% reduction in strength for a single leg stance load and a 24% reduction in strength if a falling load is applied to the hip. The data from this patient demonstrates an extremely rapid decline in bone mass and strength from radiotherapy. The decline in bone vBMD and strength in 8 weeks in this patient is comparable to what astronauts exhibit after 4 to 7 months on the International Space Station (Keyak et al., 2008; Lang et al., 2004; Lang et al., 2006).

### Example 3

### Radiation-Induced Loss of Trabecular Bone in Mice: Fundamental Variables of Dose Response, Local/Single Limb Exposure, Time Course, Sex and Stage of Skeletal Development

Animal models are critical to the understanding of biomedical disorders and the testing and development of therapies to treat diseases and disorders. Example 1 is an instance of such data that could not be collected from human cancer patients receiving radiation therapy. Though Example 2 describes a functional loss of bone in cervical cancer patients receiving radiation exposure, there are many variables associated with this newly discovered cause for bone loss that simply cannot be examined in humans. Thus, animals models (predominately mouse) have been used to examine many of these variables in the context of clinical therapy and space exploration applications. The purpose of Example 3 is to demonstrate bone loss in are the most fundamentally important variables. Many other variables are presented in Example 7.

These are: Example 3a=dose response; Example 3b=local, single limb radiation exposure v. whole body exposure; Example 3c=time course examination; Example 3d=effect of sex; Example 3e=growing mice v. skeletally mature mice.

### Common Methods:

Unless otherwise stated, all mice in Example 3 and other Examples are female C₅₇BL/6 (B6) mice between the 8 and 20 weeks of age exposed to 2 Gy X-rays (represented on graphs in White), or not irradiated (represented on graphs in Black) while under isoflurane anesthesia. Group sizes ranged from 5 to 12 and animals were grouped in a way that animal mass was similar for every group in each study. Generally the mice were humanely euthanized two weeks later (irradiated and non-irradiated control). Statistics were performed to identify significant differences with

Significance was determined using SigmaStat version 3.5 (Systat Software Inc., Richmond, Calif.). A t-test or one-way ANOVA (with LSD follow-up test) were used to identify differences between appropriate groups. The threshold for significance for all tests was set at a 5% probability of Type I error (P=0.05). Significance is generally indicated by an asterisk (*) or other symbol. Data are generally reported as mean±standard error of the mean.

In all cases, the hindlimbs were removed, and the left tibiae were evaluated for trabecular microarchitecture using microCT (microCT 20, Scanco Medical AG; Bassersdorf, Switzerland), with isotropic voxels of 9 pun/side. An approximately 1 mm section of secondary spongiosa immediately adjacent to the primary spongiosa of the proximal growth plate was scanned. A total of 100 slices were traced and evaluated. Trabecular bone parameters including bone volume fraction (BV/TV), connectivity density (Conn.D), trabecular number (Tb.N), trabecular thickness (Tb.Th), and trabecular separation (Tb.Sp), and structure model index (SMI) were quantified for all skeletal sites.

In most cases, only trabecular volume fraction and trabecular connectivity density are reported as these represent the 1) loss of bone (BV/TV), and 2) an indication of the permanence of the loss (Conn.D-generally disconnected struts do not reconnect and promote increases in bone mass.

### Results

### Example 3a

### Dose Response Study Identifies Radiation-Induced Bone Loss as a Sensitive Response

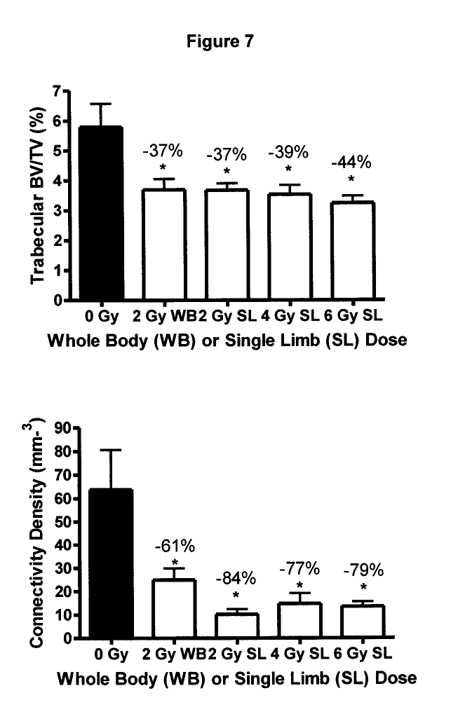

Mice 10 weeks of age received whole body doses of either 0, 2, 4 or 6 Gy X-rays. Trabecular volume fraction and Conn.D were negatively affected by radiation exposure to the same degree for all three doses. This demonstrates that a 2 Gy dose of X-rays elicits the maximum response of bone loss-irradiating with a higher dose than 2 Gy does not cause a greater amount of bone loss (FIG. 7). Compared to other biomedical disorders, this is very sensitive; a 5 Gy dose elicits the maximum response of brain lesions and a ˜10 Gy dose causes a maximum response of lung fibrosis.

### Example 3b

### Local, Single Limb Exposure Causes the Same Amount of Bone Loss as a Whole Body Dose

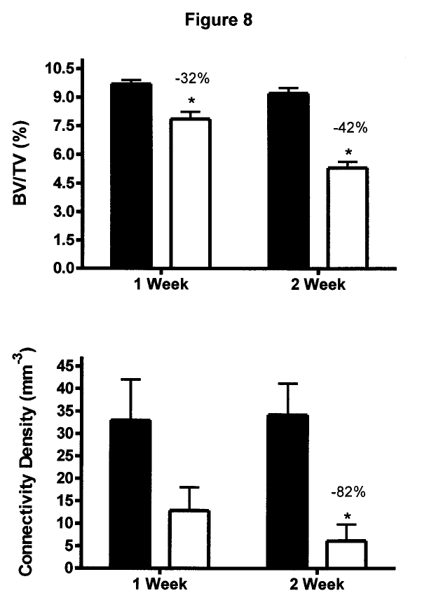

Nine-week-old mice were exposed to doses of X-rays delivered a 1) 0 Gy controls, 2) 2 Gy whole body, 3) 2 Gy right-hindlimb only, 4) 4 Gy right-hindlimb only, 5) 6 Gy right-hindlimb only. The right hindlimb was irradiated in such a way that the distal femur and whole tibia were exposed, while the left-hindlimb and entire body were shielded, and not irradiated. This study confirms the dose response results from Example 3a, 2 Gy single limb exposure results in the maximum dose response for loss of trabecular bone. The bone loss from mice with local exposure did not lose any less bone than mice exposed to a whole body dose (FIG. 8).

### Example 3c

### Radiation-Induced Bone Loss is Very Rapid

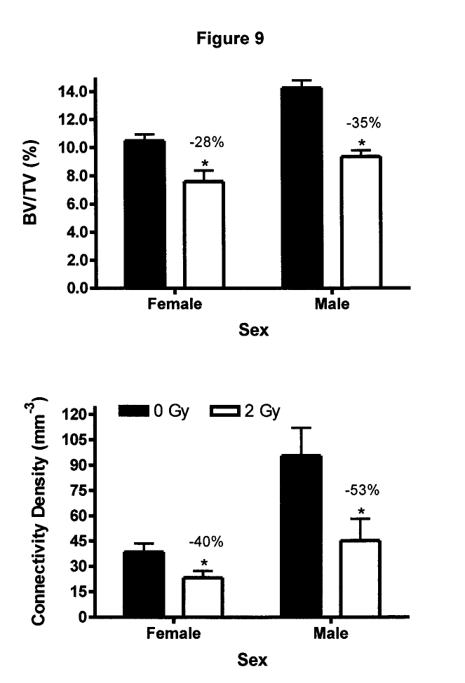

20-week-old mice were exposed to a 2 Gy whole body dose of X-rays and euthanized either 1 or 2 weeks later. Bone loss at 1 week-post exposure was as severe as bone loss 2 weeks after exposure. Radiation-induced bone loss is very rapid (FIG. 9).

### Example 3d

### Mice of Different Sexes are Both Affected by Radiation-Induced Bone Loss

To determine if sex influenced any changes in bone after irradiation, both male and female 13-week old B6 mice were exposed to a 2 Gy dose of radiation. A different response between male and female mice would suggest that hormone changes are important contributors to radiation-induced bone loss.

### Example 3e

### Growing and Skeletally Mature Mice Both Lose Bone after Radiation Exposure

Female 9-week-old (still growing) and 19 week old (skeletally mature) mice were irradiated and compared versus control. Growing bone is physiologically very different than mature bone, with chondroblasts (a radiation sensitive cell type) contributing to deposition of bone at the growth plate. Skeletally mature mice represent human adult skeletal conditions better than young, growing mice.

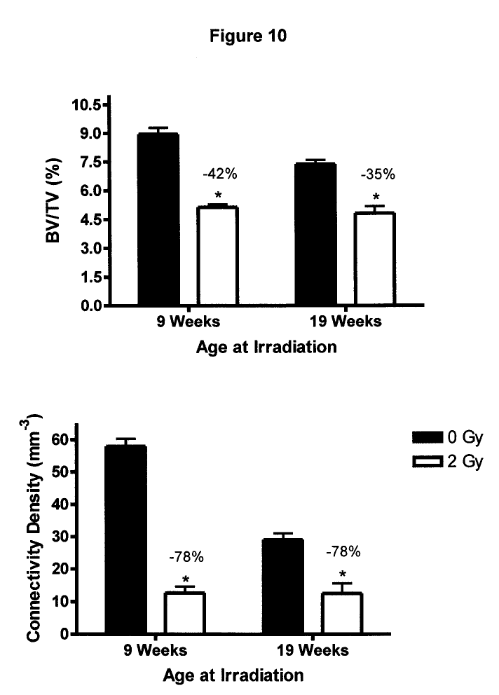

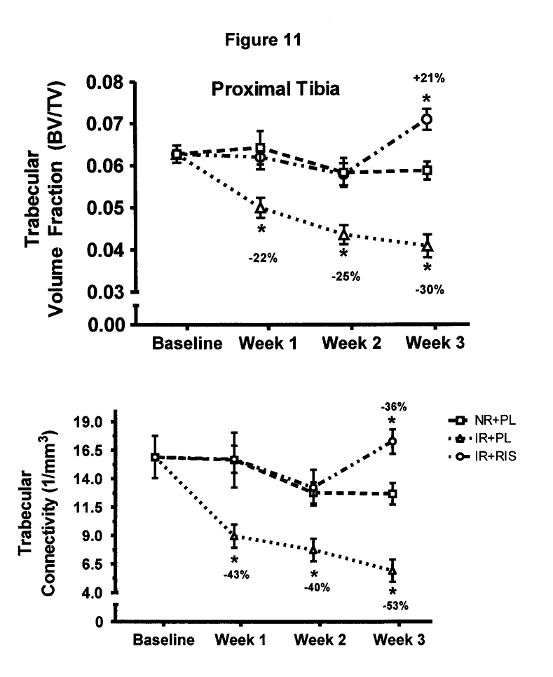

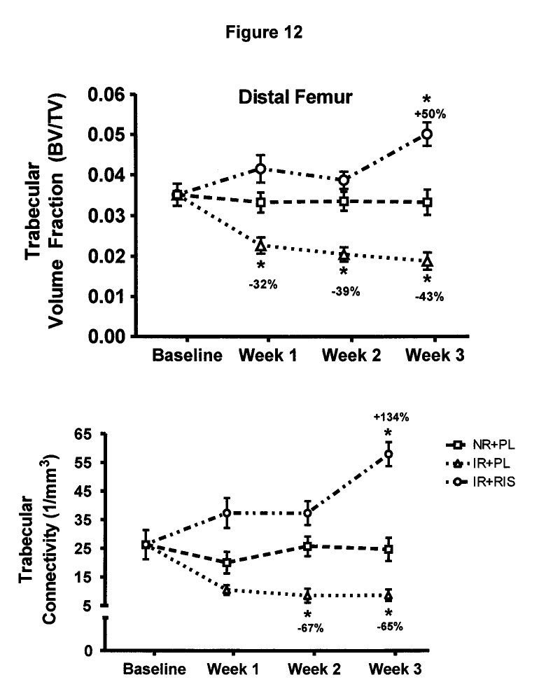

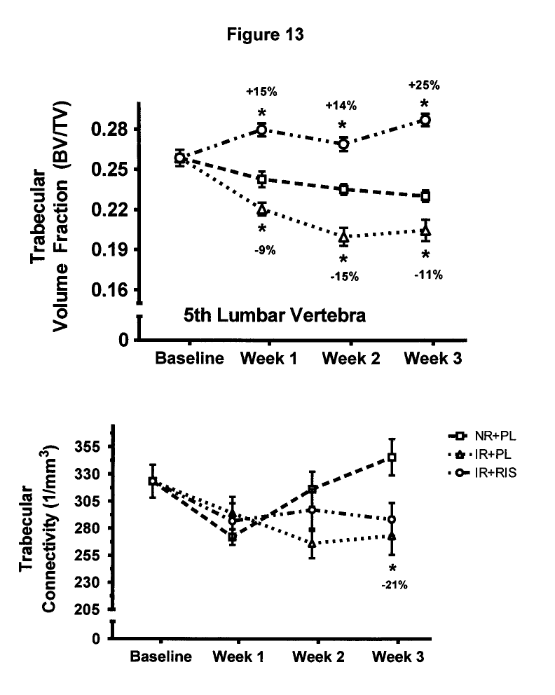

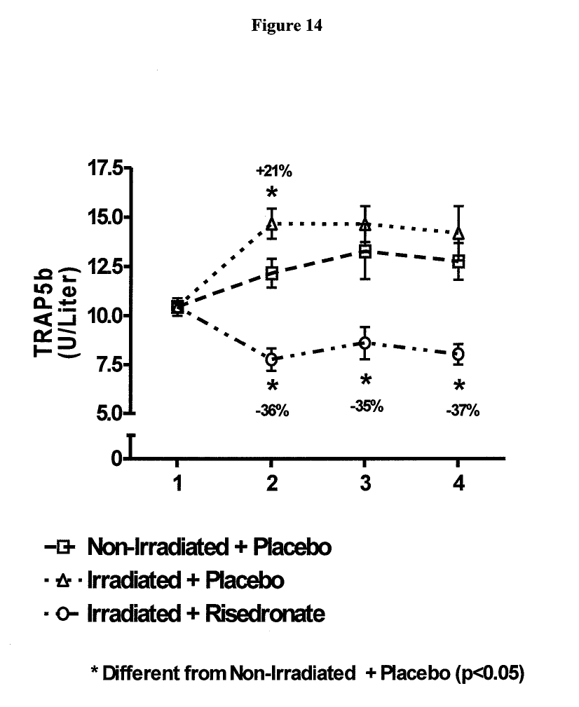

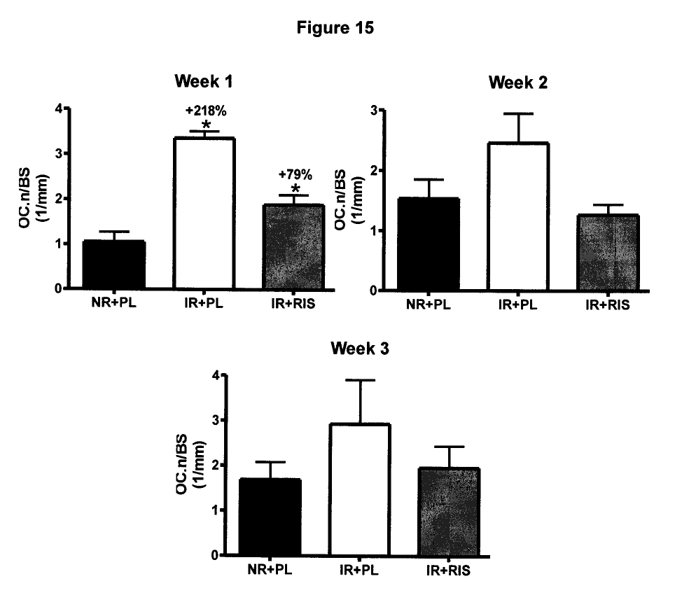

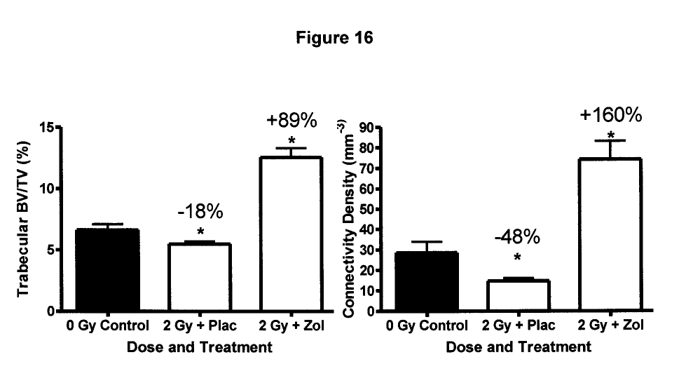

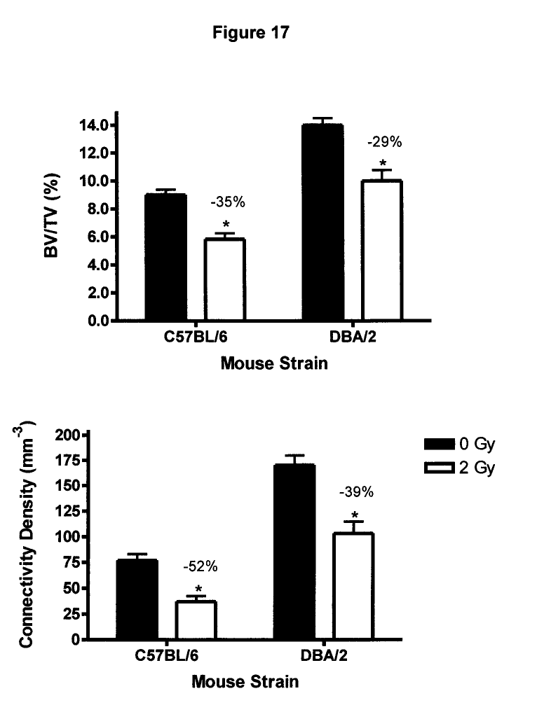

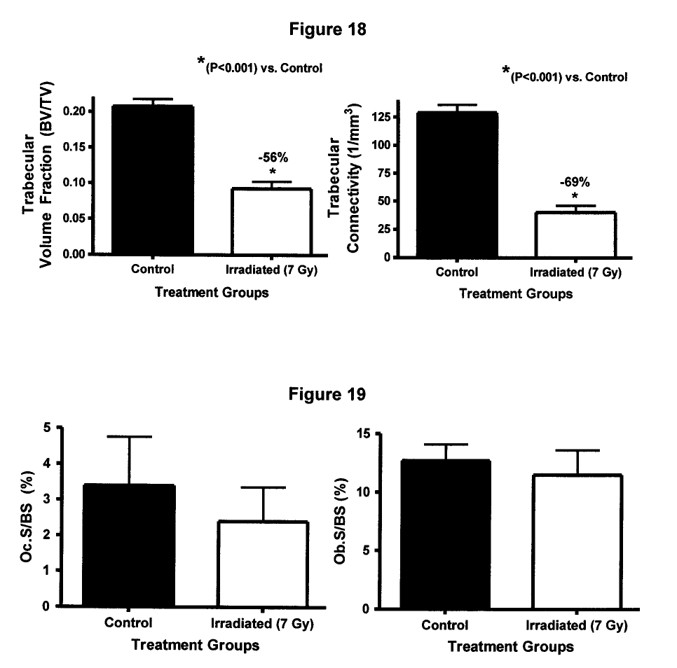

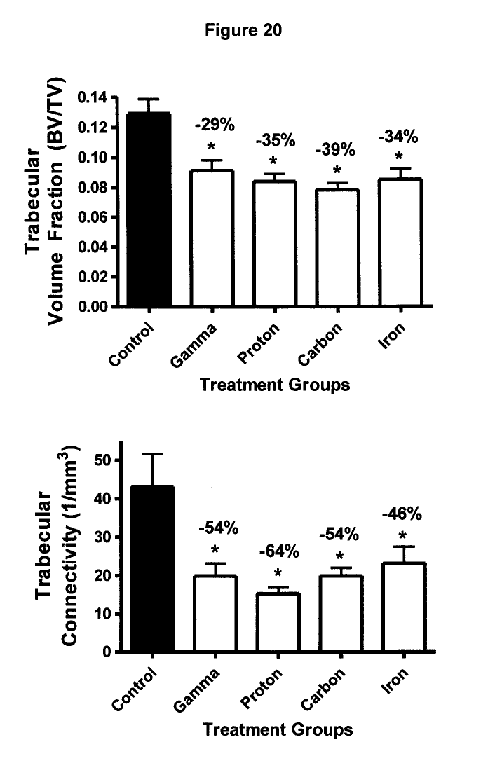

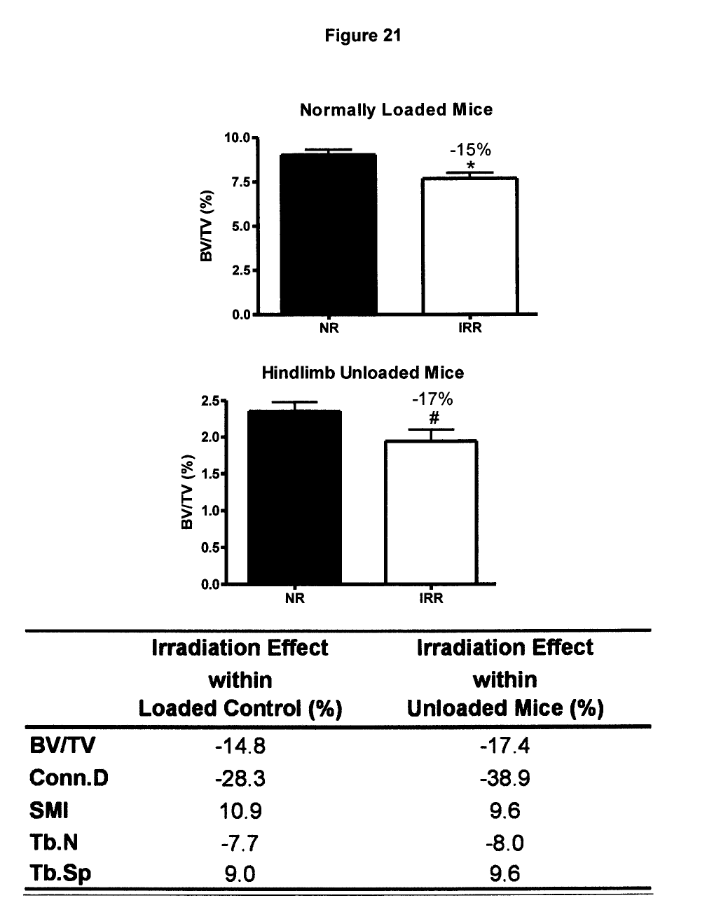

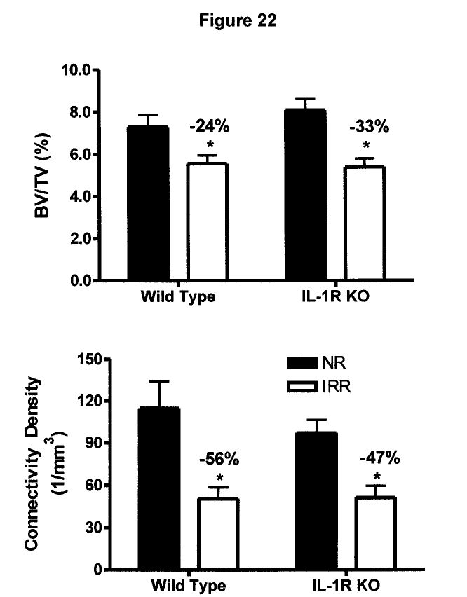

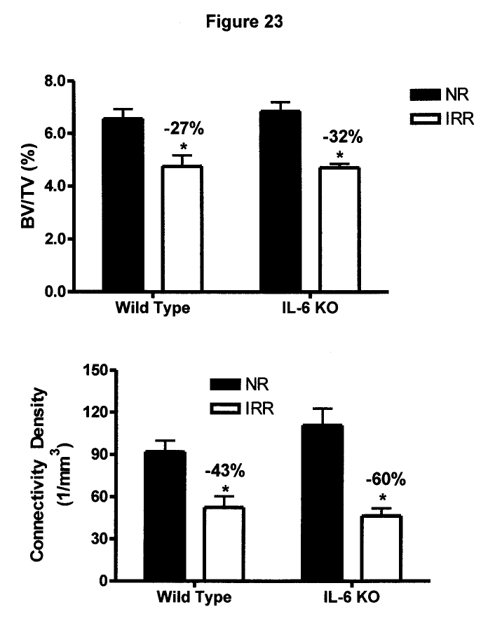

Significant and substantial reduction in bone volume fraction and other parameters of trabecular microarchitecture occur by two weeks after exposure to 2 Gy of X rays from B6 mice (Table 3). Significant reduction of these parameters relative with control were observed in both female and male B6 mice (FIG. 10), as well as from younger individuals and skeletally mature mice (FIG. 11).

TABLE 3
The percent difference for trabecular bone parameters as
determined via microCT from 2 Gy X-ray irradiated mice
versus non-irradiated control.

| | BV/TV | Conn. D. | Tb. N. | Tb. Th. | Tb. Sp |
|---|---|---|---|---|---|
| Male 2 Gy Mice (B6) | −35%* | −53%* | −14% | −0.9% | +21% |
| Vs. Cntrl. | | | | | |
| Female 2 Gy Mice | −28%* | −40%* | −11%* | −3.0% | +14%* |
| (B6) Vs. Cntrl. | | | | | |
| 9 Week-Old 2 Gy | −42%* | −78%* | −31%* | +1.0% | +50%* |
| Mice Vs Cntrl. | | | | | |
| 19 Week-old 2 Gy | −35%* | −57%* | −14%* | −7.0% | +18%* |
| Mice Vs Cntrl. | | | | | |

Notes.
Values were compared using a Student's t-test.
*P < 0.05.
Abbreviations for bone volume fraction (BV/TV); connectivity density (Conn. D.); trabecular thickness (Tb. Th.); trabecular number (Tb. N); and trabecular spacing (Tb. S.

### Example 4

### Treatment with the Antiresorptive Agent Prevents Ionizing Radiation-Induced Bone Loss in Mice

The antiresorptive bisphosphonate risedronate was selected as a representative of the class of anti-resorptive agents, and was tested for its ability to prevent radiation-induced bone loss in mice. Three different time points were examined: 1, 2 and 3 weeks after exposure. Bone loss was examined in the 5^{th} lumbar vertebra and the proximal femur in addition to the standard analysis at the proximal tibia.

### Methods:

Animals and Study Design: Twenty-week-old female C₅₇BL/6 mice (n=118 total) were examined in this study (Taconic Farms, Inc., Hudson, N.Y.). The animals were received at 15 weeks of age and allowed an acclimation period of five week prior to irradiation; food and water were available ad libitum. The Institutional Animal Care and Use Committee of Clemson University approved all procedures.

Animals were grouped to ensure similar mean body masses between groups at the outset of the study (˜23 g). Three groups were determined to receive whole body irradiation (n=72 total), with the remainder receiving no irradiation and serving as non-irradiated controls, either as a baseline group (n=10) or exposed to a sham irradiation procedure (n=36). The baseline group was sacrificed immediately prior to the irradiation procedure, and tissues were harvested as described below.

Irradiations: While under anaesthesia (1.5% isoflourane), mice were irradiated in the prone position with a single field of 140 kVp X-rays to a single-fraction mid-plane dose of 2 Gy at a rate of 1.36 Gy/min. Irradiation was performed at a nominal dose rate of 1.37 Gy/min with an exposure time of 1.46 min. A 150 kV industrial portable X-ray unit was used (Philips Medical Systems; Bothell, Wash.). Anaesthetized control mice were placed inside the inactive X-ray unit for the same amount of time as the irradiated animals, creating the sham procedure. The irradiation procedure served as the start of the experiment (Day 0).

Injections: Three of the 2 Gy irradiated groups were selected to receive subcutaneous injections of the bisphosphonate risedronate (Actonel®; Procter and Gamble Pharmaceuticals; Cincinnati, Ohio) every other day starting immediately following the irradiation procedure at a dose of 30 μg/kg/injection (IR+RIS; n=36). Equivalent volumes of PBS were injected as a placebo into the remaining 2 Gy irradiated (IR+PL; n=36) and non-irradiated (NR+PL; n=36) mice.

TABLE 4
Study Design for Example 4: Treating Radiation-Induced
Bone Loss with Risedronate.
Animal Assignment Matrix

| | Baseline | Week 1 | Week 2 | Week 3 |
|---|---|---|---|---|
| NR + PL | 10 | 12 | 12 | 11 |
| IR + PL | — | 12 | 12 | 12 |
| IR + RIS | — | 12 | 12 | 11 |
| TOTAL | 10 | 36 | 36 | 33 |

Notes:
Baseline controls euthanized at the start of the study.
NR + PL = non-irradiated treated with placebo,
IR + PL = 2 Gy whole body irradiated, treated with placebo,
IR + RIS = 2 Gy whole body irradiated, treated with 30 micrograms/kg every other day.
Groups of mice were euthanized 1, 2 or 3 weeks after irradiation.

Tissue collection: 12 individuals from each group were sacrificed at 1, 2, and 3 weeks following the initial radiation exposure. Each mouse weighed then anesthetized using isoflurane, and blood was collected for serum analysis by cardiac puncture and exsanguination followed by cervical dislocation to ensure death. Serum was isolated by centrifugation, flash frozen in liquid nitrogen, and stored at −80° C. The left and right hind limbs as well as the vertebral column were collected for analysis. Hind limbs were removed and disarticulated. Tibiae and femora were cleaned of soft tissue and fixed in a solution of 10% formalin. After 48 hours, the bones were placed in 70% ethanol. The vertebral column was frozen at −20° C. to ensure microcomputed tomography (microCT) analysis of the fifth lumbar vertebrae (L5).

Microcomputed tomography: The left tibiae were evaluated for trabecular microarchitecture using microCT (microCT 20, Scanco Medical AG; Bassersdorf, Switzerland), with isotropic voxels of 9 μm/side. An approximately 1 mm section of secondary spongiosa immediately adjacent to the primary spongiosa of the proximal growth plate was scanned. A total of 100 slices were traced and evaluated. Trabecular bone parameters including bone volume fraction (BV/TV), connectivity density (Conn.D), trabecular number (Tb.N), trabecular thickness (Tb.Th), and trabecular separation (Tb.Sp), structure model index (SMI), and volumetric bone mineral density (vBMD) were quantified for all skeletal sites.

The L5 and distal femur were also analyzed via microCT (Scanco). L5 was isolated using the microCT X-ray scout view and scanned in its entirety (˜3.5 mm) with a 10 μm voxel size. A section of the vertebral body measuring 0.5 mm immediately superior to the caudal end plate was selected for analysis. This region was chosen because of its relatively high trabecular bone density and to minimize morphological differences between samples. The left femora were evaluated in 2 regions: the distal metaphysis and mid-diaphysis. A 1 mm volume of bone immediately adjacent to the primary spongiosa of the distal growth plate was scanned and evaluated. A section of the mid-diaphysis measuring approximately 0.3 mm was scanned and evaluated to determine cortical porosity (Ct.Po) and polar moment of inertia (pMOI) within the diaphyseal bone.

Osteoclast Identification: Following tomographic analysis, the left tibiae were decalcified using a formic acid solution (Immunocal; Decal Chemical Corp., Talman, N.Y.) and embedded in a glycol methacrylate resin (ImmunoBed™; Polysciences, Wasrrington, Pa.). The samples were cut into sagittal sections with a thickness of 3 μm. A subset of each group (n=5-6) was selected for analysis. Each slide was stained with TRAP using a commercial kit (Sigma, St. Louis, Mo.) to identify osteoclasts and counterstained with hematoxylin, Radiographs assessed the earliest time point of complete decalcification. Following this, tibiae were embedded in a glycol methacrylate resin (ImmunoBed™, Polysciences; Warrington, Pa.) and cut into sagittal sections with a thickness of 1.5 um. Osteoclast presence was determined by tartrate resistant acid phosphatase (TRAP) staining of the slides using a commercial kit (Sigma; St. Louis, Mo.) and then counterstaining with hematoxylin (Sigma).

Histomorphometric evaluation was performed from captured micrographs (200×) throughout the metaphysis, starting approximately 0.25 mm distal from the growth plate (in order to exclude the primary spongiosa) and extending a further 0.5 mm. Bone histomorphometric parameters for the proximal metaphysis of the tibia were measured as described in the report of the American Society of Bone and Mineral Research (ASBMR) Histomophometry Nomenclature Committee (22). Surface measurements were quantified relative to total bone surface (BS). These measurements included osteoblast surface (Ob.S/BS; %); osteoclast surface (Oc.S/BS; %); eroded surface with the inclusion of osteoclast surface (surface covered by Howship's lacunae plus osteoclasts, [ES(Oc+)/BS], %); and eroded surface with the exclusion of osteoclast surface (surface covered by Howship's lacunae, [ES(Oc−)/BS], %). The number of osteoclasts (N.Oc) within the region of interest along trabeculae of the secondary spongiosa was also determined (N.Oc/BS, m).

Serum Chemistry: Serum samples were analyzed for circulating markers of bone formation and resorption. ELISA kits for osteocalcin (Biomedical Technologies, Inc., Stoughton, Mass.) and tartrate-resistant acid phosphatase (TRAP5b) (Immunodiagnostic Systems, Inc., Fountain Hills, Az.), respectively. The analyses were performed according to protocols provided by the manufacturers.

Statistics: All data are presented as mean±standard error of the mean. Significance was determined using SigmaStat version 3.5 (Systat Software Inc., Richmond, Calif.). A one-way ANOVA with a LSD post-hoc test was performed to differences across treatment groups and between times. The threshold for significance for all tests was set at a 5% probability of committing a Type I error (P=0.05).

### Results:

### Example 4a

### Risedronate Prevents Radiation-Induced Bone Loss at the Proximal Tibia, Distal Femur and 5^{th} Lumbar Vertebra

This study clearly demonstrates 1) risedronate prevents radiation-induced bone loss; 2) radiation-induced bone loss is not specific to the proximal tibia, but also occurs in all of the sites examined for this study (distal femur and 5^{th} lumbar vertebra); 3) confirming the very rapid bone loss observed in Example 3c.

### Example 4b

### Radiation Causes an Increase in Serum Osteoclast Markers 7 Days (but not 14 and 21 days) After Exposure-Risedronate Reduces Osteoclast Marker Levels

Serum collected from this study was examined for TRAP5b (a marker for osteoclast activity) and osteocalcin (a marker for bone formation) levels. There were no changes in osteocalcin levels from radiation, though risedronate treatment trended to decrease levels. TRAP5b levels were elevated 7 days after irradiation in placebo treated mice and reduced at all time points in risedronate treated mice.

### Example 4c

### Radiation Increases Osteoclast Surface 7 Days After Exposure in Both Placebo and Risedronate Treated Mice

Histological analysis of proximal tibia trabecular bone show a greater osteoclast surface in both IRR+Placebo and IRR+Risedronate treated mice one week, but not two and three weeks, after exposure. The increase in osteoclast surface, even with risedronate treatment show that antiresorptive doses need to be high and/or may need to proceed radiation exposure by some time period.

### Example 5

### Treatment of Ionizing Radiation-Induced Bone Loss with an Antiresorptive Agent: Rat Model Following Clinically-Modeled Radiation Dose Regimen

### Methods

As radiation has been shown to increase osteoclast number, activity, and induce bone loss in B6 mice following 2 Gy, and treatment with an antiresorptive mitigated these changes, a rat model (higher bone density, easier to compare with studies of postmenopausal osteoporosis) was used to examine radiation-induced bone loss. The right hindlimb from twenty-one week old female Sprague-Dawley rats were irradiated using a clinical irradiator (linear accelerator; LINAC; Wake Forest University). The dose applied was 16 Gy given as 4 fractions of 4 Gy each. This dose is the equivalent in terms of biological impacts on the hip during the course of radiation therapy for cancer, as determined by radiation biophysicists. Irradiations (all four total) were provided over the course of two weeks. Animals either received no radiation, radiation only, or radiation+risedronate injection (0.1 mg/kg/week subcutaneously). Administration of risedronate was provided one day prior to the first irradiation, then three times per week until sacrifice.

Animals were sacrificed two and four weeks after exposure. Hindlimbs were removed, and the tibiae were examined via microCT for several parameters. Meanings of the following trabecular parameters as they relate with conditions of increased resorption or atrophy are provided in Example 3 Methods Section: BV/TV; connectivity density (Conn.Dens.); trabecular thickness (Tb.Th.); trabecular number (Tb.N); and trabecular spacing (Tb.S.) were again determined. All measurements were taken within a region extending 1 mm distal to the growth plate.

### Results

Unlike the previous examples where percent difference from control was indicated, all mean scores±standard error are presented in Table 5.

2 Week Results: Atrophy with Radiation, Risedronate Preserves Architecture

Relative with control rats, irradiated only rats illustrated significantly reduced BV/TV, trabecular connectivity, trabecular number, and increased trabecular spacing. Therefore, by two weeks, substantial trabecular deterioration relative with control are exhibited from rats receiving radiation. This represents a very rapid loss of bone structural properties.

Treatment with risedronate (antiresorptive) largely mitigated these changes. No differences relative with control were observed for bone volume fraction, trabecular number, or trabecular spacing. Trabecular thickness was actually larger than control, which would not be indicative of compromised microarchitecture. Connectivity of the trabeculae was reduced relative with control, though with a 12% sparing effect compared with the differences that occurred after only receiving radiation. Therefore, at two weeks, administration of risedronate provided a sparing effect in terms of mitigating bone loss and preserving bone microarchitecture. Since the action of risedronate is to prevent osteoclast activity, this also provides further evidence that radiation increases bone resorption by somehow affecting osteoclasts.

4 Week Results: Atrophy Observed with Radiation Only; Architecture Preserved with Risedronate

By Week 4, irradiated only mice exhibited significant reduction of trabecular connectivity, trabecular number, and increased trabecular spacing relative with control. Therefore, the radiation-induced compromise of microarchitecture evident by Week 2 after exposure largely remains by Week 4.

In contrast, all changes or any evidence of trabecular deterioration as determined via microCT in the irradiated+risedronate group relative with control has disappeared by Week 4.

Risedronate administration again mitigated deterioration of trabecular bone in rodents (both mice from Example 4 and rats from the current example) characteristic of radiation. The dose of radiation applied in this study modeled what a human hip receives during radiotherapy, providing evidence that antiresorptive administration, which decreases osteoclast activity, can prevent osteoclast mediated bone loss during clinically modeled radiation.

TABLE 5
Mean scores for bone histomorphometric parameters as quantified via microCT
from rats exposed to clinically-modeled doses of X-rays, with or without treatment with an
antiresorptive (risedronate).

| | | Conn. Dens. | | | | |
|---|---|---|---|---|---|---|
| | BV/TV (%) | (l/mm³) | Tb. N. | Tb. Sp. (μm) | SMI | Tb. Th |
| 2 Week Examination | | | | | | |
| Control | 30.8 ± 1.3 | 122.5 ± 4.6 | 4.38 ± 0.11 | 193 ± 7 | 0.98 ± 0.11 | 81.6 ± 1.7 |
| Irradiated | 26.4 ± 1.38^{a} | 80.1 ± 16.5^{c} | 3.78 ± 0.09^{b} | 235 ± 8^{b} | 1.31 ± 0.11^{g} | 86.8 ± 1.5^{g} |
| Irradiated + Risedronate | 31.9 ± 0.79^{e} | 95.4 ± 4.4^{b} | 4.02 ± 0.11^{g} | 216 ± 8^{g} | 0.83 ± 0.07^{e} | 90.1 ± 1.4^{b} |
| 4 Week Examination | | | | | | |
| Control | 28.6 ± 1.1 | 104.2 ± 2.6 | 4.19 ± 0.09 | 203 ± 7 | 1.12 ± 0.09 | 81.2 ± 1.9 |
| Irradiated | 26.6 ± 1.2 | 87.8 ± 4.23^{a} | 3.87 ± 0.07^{a} | 227 ± 5^{a} | 1.33 ± 0.10 | 85.4 ± 1.5 |
| Irradiated + Risedronate | 34.4 ± 1.2^{b},^{f} | 109.3 ± 4.6^{e} | 4.37 ± 0.09^{f} | 193 ± 5^{f} | 0.68 ± 0.12^{a},^{f} | 90.9 ± 1.1^{c},^{h} |

Notes:
Abbreviations for bone volume fraction (BV/TV); connectivity density (Conn. Dens.); trabecular thickness (Tb. Th.); trabecular number (Tb. N); trabecular spacing (Tb. S.) and structural model index (SMI).
Values are presented mean ± SEM.
Statistics were performed using a one-way ANOVA with a Tukey follow-up test.
Italicized superscripted letters indicate P values determined from the one way ANOVA.
Superscripted a, b, and c represents significance versus control:
^{a}P < 0.05;
^{b}P < 0.01;
^{c}P < 0.001.
Superscripted d, e, and f represent significance versus irradiated only rats:
^{d}P < 0.05;
^{e}P < 0.01;
^{f}P < 0.001.
Superscripted g and h indicate trends (P < 0.1) versus control (^{g}) and irradiated only (^{h}) groups.

### Example 6

### High Doses of Zoledronate Prevent Ionizing Radiation-Induced Bone Loss

The antiresorptive bisphosphonate zoledronate was selected as representative of the class of antiresorptive agents and was tested, at high doses of 10 mg/kg, for its ability to prevent radiation-induced bone loss.

### Methods:

12-week-old female B6 mice were either 1) not irradiated and treated with a placebo injection, 2) irradiated with a 2 Gy whole body dose of X-rays and treated with a placebo injection, or 3) irradiated with a 2 Gy whole body dose of X-rays and treated with a single 10 mg/kg dose of X-rays. Bones were collected and examined by microCT 2 weeks after irradiation and zoledronate injection.

### Results:

Zoledronate prevented the bone loss. In fact, because the mice were growing and the dose of bisphosphonate was high enough bone mass was increased to greater than untreated, non-irradiated levels. This is informally called a Shenk effect—where growing mice not only have bone loss prevented but bone mass increases. The relatively high rate of turnover in young mice causes an increase in bone mass when resorption is shut down by a strong antiresorptive but bone formation continues for some period of time at normal, high levels.

### Example 7

### Ionizing Radiation-Induced Loss of Trabecular Bone in Mice: Variables of Mouse Strain, Marrow Ablating Dose, Long-Term Response with Multiple Radiation Types, Long-Term Dose Response of Protons, Protons and Disuse, and Inflammatory Cytokine Knockout Mice

The present example examines many variables for radiation-induced bone loss that have applicability for both clinical therapy and space exploration applications. Examples 1 to 6 are principally relevant, but the present data represent many variables examined as part of the process of understanding why and for what variables radiation causes bone loss. The common methods are generally the same as described in Example 3.

### Example 7a

### Radiation Results in Functional Bone Loss 2 Weeks in both C₅₇BL/6 and DBA/2

Female C₅₇BL/6 (B6) and DBA/2 mice were exposed to 2 Gy X-rays. Significant and substantial reduction in bone volume fraction and other parameters of trabecular microarchitecture occur by two weeks after exposure to 2 Gy of X rays in both strains of mice to approximately the same degree.

### Example 7b

### Bone Loss After Exposure to a Marrow Ablating Dose of X-rays

Eight week-old female B6 mice were exposed to a 7 Gy whole body dose of gamma-rays (cobalt source), a dose that is LD 50/30 (a lethal dose for 50% of the mice 30 days after exposure). This models the ablation of marrow that is necessary for bone marrow transplantation. Mice were humanely euthanized two weeks after exposure (before lethality) and examined by microCT and histology.

### Example 7c

### Multiple Radiation Types, Representing Solar and Cosmic Sources, Cause a Long-Term Loss of Bone Mass

Trabecular bone was irradiation with 2 Gray (Gy) dose of gamma, proton, carbon or iron radiation. Bones were examined in female B6 mice (9 weeks of age at exposure) four months after exposure by microCT as previously described. For this study, proton radiation models exposure to a solar particle event and carbon and iron radiation represent exposure to galactic and cosmic radiation.

When the proximal tibia was measured 1 mm distal to growth plate 4 months after irradiation, the bone showed a reduction in both trabecular volume fraction (FIG. 4) and density connectivity (FIG. 5). Decreased trabecular bone volume (29-39%) was detected two weeks after initiating exposure. Decreased trabecular connectivity (46-64%) was detected two weeks after initiating exposure. This degree of loss, particularly accompanied by loss of trabecular connectivity four months after exposure strongly suggest permanent deficits in bone quantity and quality.

### Example 7d

### Short and Long-Term Radiation-Induced Loss of Bone Mass with Proton and X-ray Doses of 1 Gy

In these two experiments mice were exposed to doses lower than 2 Gy to examine a dose response. For the short-term study 13 week old female B6 mice were exposed to 1 Gy of x-rays and two weeks after exposure bone loss was compared to non-irradiated controls with the previously discussed microCT analysis technique. For the long-term study 9 week old female B6 mice were exposed to proton doses of 0.5, 1 and 2 Gy and bones collected for microCT analysis (study design-strain, age, sex and study duration—were chosen to match that of Example 5.

A nearly significant reduction in trabecular volume fraction (BV/TV) (but no difference in other parameters) two weeks after exposure to 1 Gy of x-rays from B6 mice. For the long-term examination of bone after exposure to protons, significant declines in BV/TV and loss of Tb.Sp were observed for both the 2 Gy and 1 Gy exposed groups

TABLE 6
The percent difference for trabecular bone parameters as determined
via microCT from 1 Gy X-ray irradiated mice versus non-irradiated
control (2 weeks post-exposure); or 0.5, 1 or 2 Gy irradiated mice
versus non-irradiated controls (4 months post-exposure).

| | | Conn. | | | |
|---|---|---|---|---|---|
| | BV/TV | D. | Tb. N. | Tb. Th. | Tb. Sp |
| 1 Gy x-rays 2 weeks | −11.9%# | −20.1% | −3.3% | +0.9% | +5.4% |
| post exposure | | | | | |
| 0.5 Gy x-rays 4 | +5.1%* | 0% | −4.5% | +2.9% | +7.1% |
| months post exposure | | | | | |
| 1 Gy x-rays 4 months | −13%* | +16% | −6.1% | −5.3% | +9.0%\hat{ } |
| post exposures | | | | | |
| 2 Gy x-rays 4 months | −20%* | −7.5% | −9.0%\hat{ } | +2.3% | +11%* |
| post exposures | | | | | |

Notes:
For the short term study with x-ray exposure values were compared using a Student's t-test.
^{#}P = 0.07 (trend);
For the long-term study with proton exposure values were compared using a one-way-ANOVA with SNK follow-up
*P < 0.05;
\hat{ }P = 0.09.
Abbreviations for bone volume fraction (BV/TV); connectivity density (Conn. D.); trabecular thickness (Tb. Th.); trabecular number (Tb. N); and trabecular spacing (Tb. S.)

### Example 7e

### Radiation Causes Bone Loss when Combined with Disuse

Astronauts on long-duration lunar missions will be exposed to a complex spaceflight environment, including microgravity and radiation. The negative effects of microgravity on the skeletal system have been well documented. In addition, recent studies have documented that doses as low as 1 Gy of proton radiation, representing a solar particle event, lead to long-term bone loss in mice. However, the combined effect of radiation and unloading has not been examined. The present study investigated the effects on the skeletal system of proton radiation followed by unloading. Sixteen-week-old female C₅₇BL/6 mice (n=15/group; 4 groups) were exposed to either 1 Gy of 250 MeV protons (IRR) or served as non-irradiated controls (NR). One day after exposure, half the irradiated mice and half the control mice were hindlimb suspended (HLS), with the remainder serving as normally loaded controls (LC). Mice were killed after 4 weeks of unloading. Tibiae and femora were analyzed via microcomputed tomography, mechanical testing, mineral composition, and histology. Radiation treatment alone resulted in a significant loss of bone and deterioration of trabecular microarchitecture in the tibia and femur at 4 weeks including trabecular bone volume fraction (BV/TV) and other parameters, with no effect on cortical bone. HLS alone induced substantial deterioration of both trabecular and cortical bone in the tibia and femur, with corresponding decreases in cortical bone strength. Histology and serum chemistry indicated increased resorption in HLS animals, with no significant differences in irradiated animals. HLS+IRR resulted in generally lower values for BV/TV, connectivity density (Conn.D), trabecular separation (Tb.Sp), trabecular number (Tb.N), and structural model index (SMI) than both HLS and IRR independently. Overall, the combination of IRR+HLS resulted in greater bone loss and deterioration of trabecular microarchitecture than the two challenges separately, and appear to be additive. Future models of irradiation and unloading would benefit from using shorter durations of hindlimb suspension, more appropriately modeling the skeletal challenges of combined microgravity and radiation experienced by astronauts in the spaceflight environment.

### Example 7f

### Mice Lacking the Genes for Interleukin-1 Receptor (IL-1R) and Interleukin-6 (IL-6) are as Sensitive to Radiation-Induced Bone Loss as Wild Type (WT-normal) Mice

As part of the process of identifying the molecular causes for radiation-induced bone loss, mice with genes related to inflammatory cytokines were examined. Female mice with the B6 background strain (wild type) were delivered with IL-R or IL-6 knocked out. They were irradiated with the standard 2 Gy whole body dose and humanely euthanized 2 weeks later.

### Example 8

### Standard Treatment Regimens for Different Types of Cancer

The present example describes the types of cancer for which radiation therapy may be used and for which the present method of treating or preventing radiation-induced bone loss (osteoclast activation) can be applied.

### Endometrial Cancer:

Definition of endometrial cancer: Cancer that forms in the tissue lining the uterus (the small, hollow, pear-shaped organ in a woman's pelvis in which a baby grows). Most endometrial cancers are adenocarcinomas (cancers that begin in cells that make and release mucus and other fluids).
Estimated new cases and deaths from endometrial (uterine corpus) cancer in the United States in 2007:

New cases: 39,080

Deaths: 7,400

### Survival:

When all cases of endometrial cancer are looked at together, the 5-year relative survival rate is 84%. For cancer found at an early stage, the survival rate is much higher.

### Treatments by Stage:

### Stage I Endometrial Cancer

Treatment of stage I endometrial cancer may include the following:
Surgery (total abdominal hysterectomy and bilateral salpingo-oophorectomy). Lymph nodes in the pelvis and abdomen may also be removed for examination under a microscope to check for cancer cells.
Surgery (total abdominal hysterectomy and bilateral salpingo-oophorectomy, with or without removal of lymph nodes in the pelvis and abdomen) followed by internal radiation therapy or external radiation therapy to the pelvis. After surgery, a plastic cylinder containing a source of radiation may be placed in the vagina to kill any remaining cancer cells.
Radiation therapy alone for patients who cannot have surgery.
Clinical trials of radiation therapy and/or chemotherapy.

### Stage II Endometrial Cancer

Treatment of stage IIA endometrial cancer is usually a combination of therapies, including internal and external radiation therapy and surgery.

### Stage IIA

Treatment of stage IIA endometrial cancer may include the following:
Surgery (total abdominal hysterectomy and bilateral salpingo-oophorectomy). Lymph nodes in the pelvis and abdomen may also be removed for examination under a microscope to check for cancer cells.
Surgery (total abdominal hysterectomy and bilateral salpingo-oophorectomy, with or without removal of lymph nodes in the pelvis and abdomen) followed by internal radiation therapy or external radiation therapy to the pelvis. After surgery, a plastic cylinder containing a source of radiation may be placed in the vagina to kill any remaining cancer cells.
Radiation therapy alone for patients who cannot have surgery.
Clinical trials of radiation therapy and/or chemotherapy.
   This summary section refers to specific treatments under study in clinical trials, but it may not mention every new treatment being studied. Information about ongoing clinical trials is available from the NCI Web site.

### Stage IIB

Treatment of stage IIB endometrial cancer may include the following:
Surgery (total abdominal hysterectomy, bilateral salpingo-oophorectomy, and removal of lymph nodes in the pelvis and abdomen for examination under a microscope to check for cancer cells) followed by radiation therapy.
Internal radiation therapy and external radiation therapy, followed by surgery (hysterectomy and bilateral salpingo-oophorectomy, and removal of lymph nodes in the pelvis and abdomen for examination under a microscope to check for cancer cells).
Surgery (radical hysterectomy with or without removal of lymph nodes in the pelvis for examination under a microscope to check for cancer cells).

### Stage III Endometrial Cancer

Treatment of stage III endometrial cancer may include the following:
Surgery (radical hysterectomy and removal of lymph nodes in the pelvis for examination under a microscope to check for cancer cells) followed by internal radiation therapy and external radiation therapy.
Radiation therapy alone for patients who cannot have surgery.
Hormone therapy for patients who cannot have surgery or radiation therapy.
Clinical trials of chemotherapy.
Clinical trials of new therapies.

### Stage IV Endometrial Cancer

Treatment of stage IV endometrial cancer may include the following:
Internal radiation therapy and external radiation therapy.
Hormone therapy.
Clinical trials of chemotherapy.
   Some women with Stage I, II, or III uterine cancer need both radiation therapy and surgery. They may have radiation before surgery to shrink the tumor or after surgery to destroy any cancer cells that remain in the area. Also, the doctor may suggest radiation treatments for the small number of women who cannot have surgery.

### Cervical Cancer:

Definition of cervical cancer: Cancer that forms in tissues of the cervix (the organ connecting the uterus and vagina). It is usually a slow-growing cancer that may not have symptoms but can be found with regular Pap tests (a procedure in which cells are scraped from the cervix and looked at under a microscope).
Estimated new cases and deaths from cervical (uterine cervix) cancer in the United States in 2007:

New cases: 11,150

Deaths: 3,670

Some Form of Radiation is Used in Stage 1-4. Internal Radiation May be Used in Stage 0 if Surgery Cannot be Performed. Internal Radiation is Used in Stage 1A and Both Internal and External Radiation is Used in Stage 1B and Higher.
Stages and treatment: 5 stages that are broken down into sub-stages
Cervical Cancer is diagnosed and staged by: Yes, chemo typically follows radiation however, research supports using chemoradiation (both administered at the same time to improve outcomes.) With the exception of breast and endometrial cancer, there is no biological evidence that HRT may increase the recurrence risk.
http://content.karger.com/ProdukteDB/produkte.asp?Aktion=ShowPDF&ArtikelNr=95727 &ProduktNr=224165&filename=95727.pdf

### Stage 0 Carcinoma in Situ

In stage 0, abnormal cells are found in the innermost lining of the cervix. These abnormal cells may become cancer and spread into nearby normal tissue. Stage 0 is also called carcinoma in situ.

### Treatment Options:

op electrosurgical excision procedure (LEEP).
Laser surgery.
Conization.
Cryosurgery.
Total hysterectomy for women who cannot or no longer want to have children.
Internal radiation therapy for women who cannot have surgery.
   Stage 1A Survival:. The five-year survival rate ranges from 96 to 99 percent.
   Stage IA: A very small amount of cancer that can only be seen with a microscope is found in the tissues of the cervix. Stage IA is divided into stages IA1 and IA2, based on the size of the tumor.
In stage IA1, the cancer is not more than 3 millimeters deep and not more than 7 millimeters wide.
In stage IA2, the cancer is more than 3 but not more than 5 millimeters deep, and not more than 7 millimeters wide.

### Treatment Options are:

Total hysterectomy with or without bilateral salpingo-oophorectomy.
Conization.
Radical hysterectomy and removal of lymph nodes.
Internal radiation therapy.
   Stage 1B Survival: Five-year survival rates for this stage of cervical cancer are 80 to 90 percent.
   Stage IB: In stage. IB, cancer can only be seen with a microscope and is more than 5 millimeters deep or more than 7 millimeters wide, or can be seen without a microscope. Cancer that can be seen without a microscope is divided into stages IB1 and IB2, based on the size of the tumor.
In stage 1B1, the cancer can be seen without a microscope and is not larger than 4 centimeters.
In stage IB2, the cancer can be seen without a microscope and is larger than 4 centimeters.

### Treatment Options are:

A combination of internal radiation therapy and external radiation therapy.
Radical hysterectomy and removal of lymph nodes.
Radical hysterectomy and removal of lymph nodes followed by radiation therapy plus chemotherapy.
Radiation therapy plus chemotherapy.
A clinical trial of high-dose internal radiation therapy combined with external radiation therapy.
   Stage 2 Survival: Five-year survival is 65 to 69 percent.
   In stage II, cancer has spread beyond the cervix but not to the pelvic wall (the tissues that line the part of the body between the hips) or to the lower third of the vagina. Stage II is divided into stages IIA and IIB, based on how far the cancer has spread.
Stage IIA: Cancer has spread beyond the cervix to the upper two thirds of the vagina but not to tissues around the uterus.

### Treatment Options are:

Treatment of stage IIB cervical cancer may include internal and external radiation therapy combined with chemotherapy.
Stage 3 Survival: Five-year survival is 40 to 43 percent.

### Stage 3A

Stage IIIA: Cancer has spread to the lower third of the vagina but not to the pelvic wall.

### Treatment Options:

Treatment of stage III cervical cancer may include internal and external radiation therapy combined with chemotherapy.

### Stage 3B

Stage IIIB: Cancer has spread to the pelvic wall and/or the tumor has become large enough to block the ureters (the tubes that connect the kidneys to the bladder). This blockage can cause the kidneys to enlarge or stop working. Cancer cells may also have spread to lymph nodes in the pelvis.

### Treatment Options:

Treatment of stage III cervical cancer may include internal and external radiation therapy combined with chemotherapy.
Stage 4A The five-year survival rate for this stage of cancer is 15 to 20 percent.
In stage IV, cancer has spread to the bladder, rectum, or other parts of the body. Stage IV is divided into stages WA and IVB, based on where the cancer is found.
Stage IVA: Cancer has spread to the bladder or rectal wall and may have spread to lymph nodes in the pelvis.

### Treatment Options:

Treatment of stage III cervical cancer may include internal and external radiation therapy combined with chemotherapy.

### Stage 4B

Stage IVB: Cancer has spread beyond the pelvis and pelvic lymph nodes to other places in the body, such as the abdomen, liver, intestinal tract, or lungs.

### Treatment Options:

Radiation therapy as palliative therapy to relieve symptoms caused by the cancer and improve quality of life.
Chemotherapy.
Clinical trials of new anticancer drugs or drug combinations.

### Internal Radiation/Brachytherapy

Brachytherapy refers to radiation that is given from a short distance, in contrast to external beam radiation, or teletherapy, which is given from a longer distance. In the majority of these treatments radiation applicators are placed within a cavity of the body such as the bronchus (airway) in the lung, esophagus, vagina, cervix or uterus. The primary advantage of internal radiation is the ability to deliver a higher radiation dose while the normal tissues receive less radiation since the radiation sources are placed within the tumor. Brachytherapy has been used to cure cervical cancer since the beginning of the century. This successful treatment for gynecologic malignancies was developed in Europe by a number of institutions. Both radium and cesium have been used as radioactive sources to give the internal radiation. Low dose rate (LDR) brachytherapy was implemented as the first internal radiation system. Low dose rate brachytherapy refers to radiation that is delivered slowly, or at a low dose rate. In order to prescribe a radiation dose that will eliminate the cancer, the instruments need to be in place for an extended period of time. Therefore, patients stay in the hospital with applicators in the gynecologic tract for 2 to 3 days. For cervical cancer patients, the procedure is repeated one week later. High dose rate brachytherapy refers to radiation that is given very rapidly. In contrast to low dose rate brachytherapy where treatments require 2 to 3 days, HDR brachytherapy is delivered over minutes. In order to prevent potential complications from HDR brachytherapy, multiple insertions are required. For cervical cancer patients, 5 insertions has become the standard of care. Although 5 insertions in HDR brachytherapy may appear to be less convenient than 2 LDR insertions, the total time that applicators are in the gynecologic tract (vagina, cervix and/or uterus) for each insertions is on average 2½ hours for the HDR approach in contrast to 50 hours for the LDR approach. For endometrial cancer patients that are receiving brachytherapy alone or in combination with external beam radiation after a hysterectomy, a total of 2 insertions are used in which each insertion lasts about 1 hour. http://wwww.humonc.wisc.edu/modules/mediawiki/index.php/Cervical_brachytherapy#Computer_Calculations
Intracavitary brachytherapy at low dose rate (LDR), often with the addition of external-beam radiotherapy, has long been considered the treatment of choice for carcinoma of the cervix, maximizing acute damage in the treatment volume, whilst minimizing late effects. In recent years, primarily for reasons of convenience and cost, there has been a move towards treatments involving a few fractions at high dose rate (HDR). Using data from cells of human origin cultured in vitro, we make estimates of the doses that, delivered in 2-12 HDR fractions, produce tumour control and early effects equivalent to intracavitary treatments at LDR. We also show that, for situations where the normal-tissue dose responsible for late effects is significantly smaller than the tumour dose, HDR schemes may be devised which, while yielding early killing comparable with that of LDR, should not result in worse late effects. We suggest that this scenario probably applies to treatment of carcinoma of the cervix.
http://bjr.birjournals.org/cgi/content/abstract/64/758/133?ck=nck

### External Dose Example:

Patients received external beam radiation using megavoltage machines (Co⁶⁰ or lineal accelerator equipment) with a minimum photon-beam energy of 2.25 MV with an isocenter technique to the whole pelvis for a total dose of 50 Gy (5 weeks, 2 Gy fractions from Monday to Friday) followed by one or two intracavitary Cesium (low-dose rate) applications within 2 weeks of finishing external radiation. The planned total dose to point A was at least 85 Gy. Patients were treated with the conventional 4-field box technique. Irradiated volume was to include the whole uterus, paracervical, parametrial, and uterosacral regions, as well as external iliac, hypogastric, and obturator lymph nodes. http://www.pubmedcentral.nih.gov/articlerenderfcgi?artid=1420274 Internal Dose Unable to find examples of dosing. There is a computer program that calculates this amount and it varies depending on location and size of tumor.

### Breast Cancer:

### Sequence of Treatment:

Usually surgery is first.
If chemotherapy is going to be part of your care, it often goes second.
Radiation usually follows surgery and chemotherapy (when chemotherapy is given). Radiation therapy (as part of breast-conserving local therapy) consists of postoperative external-beam radiation therapy (EBRT) to the entire breast with doses of 45 Gy to 50 Gy, in 1.8 Gy to 2.0 Gy daily fractions over a 5-week period. Shorter hypofractionation schemes achieve comparable results. A further radiation boost is commonly given to the tumor bed. Two randomized trials, conducted in Europe have shown that using boosts of 10 Gy to 16 Gy reduces the risk of local recurrence from 4.6% to 3.6% at 3 years (P=0.044), [Level of evidence: liiDii] and from 7.3% to 4.3% at 5 years (P<0.001), respectively. [Level of evidence: liiDii] If a boost is used, it can be delivered either by EBRT, generally with electrons, or by using an interstitial radioactive implant.
Tamoxifen or other hormonal (anti-estrogen) therapy is most commonly started after the other treatments have been given.
   There are many exceptions to this sequence, however. For women with stage III or IV disease, chemotherapy may be given first to shrink large tumors and address cancer in the rest of the body, before major surgery. Some centers or clinical studies give chemotherapy and radiation together (not separately). There are also many other variations in timing and sequence.
   http://www.breastcancer.org/treatment/planning/sequence.jsp

### Local Treatment:

“Local” treatment refers to anything that is targeted to a specific area of the body—such as the breast, the lymph nodes, the lungs—as opposed to the whole body. This includes surgery.

### Systemic Treatment: the Whole Body

The goal of systemic therapy is to get rid of any cancer cells that may have spread to another part of the body. It's an “insurance policy” that may be used even if there is no direct proof that cancer has spread. If the cancer HAS spread and formed tumors elsewhere, systemic treatment can help shrink the cancer and, it is hoped, lead to remission. Systemic treatment decisions are made based on “personality features” of the cancer. The “meaner” the cancer's personality, the higher the risk of cancer spread, and the greater the need for systemic treatment. The milder the personality, the lower the risk of spread, and the smaller the need for systemic management.

There are four main types of systemic therapy:
1. Hormonal (anti-estrogen) therapies are medicines usually given by pill or, less commonly, by injection under the skin. These medications either 1) reduce the amount of estrogen in your body, or 2) block estrogen's effects, in order to inhibit cancer cell growth throughout your body.
2. Chemotherapies are medicines given by pill or directly into the bloodstream (through a needle or port) that destroy cancer cells. Chemotherapy works by interfering with the cancer cells' ability to reproduce and function from day to day. J. Immune therapy is a very new area of medicine that attempts to use or imitate the body's own system for fighting disease, to defeat the cancer. The name immune therapy comes from the immune system. The goal may be “active immunity” to stimulate or trick the body's defenses into blocking or counteracting cancer, cell activity. Vaccines fall into this category. Or the goal may be “passive immunity,” which involves giving the body a fighting protein or “antibody” it lacks, so that the immune system can do its job against the cancer. The name “passive” is used because the body isn't required to do the fighting work.
   Currently, only one immune therapy, Herceptin, is widely available. It is given directly into the bloodstream (through a needle or port). Herceptin is only appropriate for women with advanced breast cancer who have a particular cancer gene, called HER2/neu, that is overactive or is being “overexpressed.” Herceptin is an example of a “passive immunity” therapy. Special immune proteins (antibodies) in the medication find and stop the bad-acting proteins made by the HER2/neu cancer genes. Halting this protein action brings cancer cell growth under better control.
   With more research, vaccines that work with the immune system in different ways for a wider range of women and cancer types—will become available.
4. Anti-angiogenesis therapies halt the growth of new blood vessels that bring nutrients to the cancer cells—in other words, you “starve” the tumor of things it needs to grow and survive. Currently, these treatments are available only in clinical trials, on a very limited basis.

### Stages:

### Stage 0

This stage is used to describe non-invasive breast cancer. There is no evidence of cancer cells breaking out of the part of the breast in which it started, or of getting through to or invading neighboring normal tissue. LCIS and DCIS are examples of stage 0.

### Stage I

This stage describes invasive breast cancer (cancer cells are breaking through to or invading neighboring normal tissue) in which
The tumor measures up to two centimeters, AND
No lymph nodes are involved.

### Stage II

This stage describes invasive breast cancer in which:
The tumor measures at least two centimeters, but not more than five centimeters, OR
Cancer has spread to the lymph nodes under the arm on the same side as the breast cancer. Affected lymph nodes have not yet stuck to one another or to the surrounding tissues, a sign that the cancer has not yet advanced to stage III. (The tumor in the breast can be any size.)

### Stage III

Stage III is divided into subcategories known as IIIA and IIIB.

### Stage IIIA

Stage IIIA describes invasive breast cancer in which:
the tumor measures larger than five centimeters, OR
there is significant involvement of lymph nodes. The nodes clump together or stick to one another or surrounding tissue.

### Stage IIIB

This stage describes invasive breast cancer in which a tumor of any size has spread to the breast skin, chest wall, or internal mammary lymph nodes (located beneath the breast right under the ribs, inside the middle of the chest).
Stage IIIB includes inflammatory breast cancer, a very uncommon but very serious, aggressive type of breast cancer. The most distinguishing feature of inflammatory breast cancer is redness involving part or all of the breast. The redness feels warm. You may see puffiness of the breast's skin that looks like the peel of a navel orange (“peau d'orange”), or even ridges, welts, or hives. And part or all of the breast may be enlarged and hard. A lump is present only half of the time. Inflammatory breast cancer is sometimes misdiagnosed as a simple infection.

### Stage IV

This stage includes invasive breast cancer in which
a tumor has spread beyond the breast, underarm, and internal mammary lymph nodes, and
a tumor may have spread to the supraclavicular lymph nodes (nodes located at the base of the neck, above the collarbone), lungs, liver, bone, or brain.
   “Metastatic at presentation” means that the breast cancer has spread beyond the breast and nearby lymph nodes, even though this is the first diagnosis of breast cancer. The reason for this is that the primary breast cancer was not found when it was only inside the breast. Metastatic cancer is considered stage IV.

### Additional Staging Information:

You may also hear terms such as “early” or “earlier” stage, “later” or “advanced” stage breast cancer. Although these terms are not medically precise (they may be used differently by different doctors), here is a general idea of how they apply to the official staging system:

### Early Stage:

Stage 0
Stage I
Stage II
   Later stage:
(stage II if there are many lymph nodes involved)
Stage III (IIIA, IIIB)

### Advanced Stage:

Stage IV
   You may also hear the cancer described by three characteristics:
size (T stands for tumor),
node involvement (N stands for node), and
whether it has metastasized (M stands for metastasis).
   The T category describes the original (primary) tumor:
TX means the tumor can't be measured or found.
T0 means there isn't any evidence of the primary tumor.
Tis means the cancer is “in situ” (the tumor has not started growing into the breast tissue).
The numbers T1-T4 describe the size and/or how much the cancer has grown into the breast tissue. The higher the T number, the larger the tumor and/or the more it may have grown into the breast tissue.
   The N category describes whether or not the cancer has reached nearby lymph nodes:
NX means the nearby lymph nodes can't be measured or found.
N0 means nearby lymph nodes do not contain cancer.
The numbers N1-N3 describe the size, location, and/or the number of lymph nodes involved. The higher the N number, the more the lymph nodes are involved.
   The M category tells whether there are distant metastases (whether the cancer has spread to other parts of body):
MX means metastasis can't be measured or found.
M0 means there are no distant metastases.
M1 means that distant metastases were found.
   Once the pathologist knows your T, N, and M characteristics, they are combined, and an overall “stage” of 0, I, II, III, IIIA, IIIB, or IV is assigned.
   For example, a T1, N0, M0 breast cancer would mean that the primary breast tumor:
is less than two centimeters across (T1),
does not have lymph node involvement (N0), and
has not spread to distant parts of the body (M0).
   This cancer would be grouped as a stage I cancer.

### Treatment Options

STAGE 0 non-invasive

### Local Treatment

to the breast area Total mastectomy (radiation after mastectomy rarely needed)

### OR

Lumpectomy+radiation

### OR

Lumpectomy alone with or without “internal” radiation—only for a limited subset of women

### Local Treatment

to the lymph node area None required

### Local Treatment

to other parts of the body (applies only to metastatic disease) Does not apply

### Systemic Treatment

Hormonal (anti-estrogen) May be used for local benefits against new or recurrent breast cancer

### Systemic Treatment

ChemotherapyNone requiredSystemic Treatment
Immune therapyNo current role
Treatment OptionsSTAGE II invasive
(>2 cm<5 cm; OR lymph nodes involved)
Local Treatment to the breast areaModified radical mastectomy; radiation after surgery may be needed

### OR

Lumpectomy+radiation for one site of cancer <4 cm that is completely removed

### OR

Chemotherapy to shrink a large single cancer, followed by lumpectomy+radiation

### Local Treatment

to the lymph node area
Axillary lymph nodes removed by traditional approach OR sentinel approach (for women without enlarged nodes)

### AND

Possible radiation to supraclavicular and/or internal mammary lymph nodes, IF axillary nodes are involved

### Local Treatment

to other parts of the body (applies only to metastatic disease) Does not apply

### Systemic Treatment

Hormonal (anti-estrogen) May be used for local and systemic benefits

### Systemic Treatment

ChemotherapyCommonly recommended

### Systemic Treatment

Immune therapyAvailable for some women in clinical trials
Treatment Options STAGE IIIA invasive (>5 cm OR lymph nodes involved and clumped together)
Local Treatment to the breast areaModified radical mastectomy followed by radiation

### OR

Chemotherapy to shrink a large single cancer, followed by lumpectomy+radiation

### Local Treatment

to the lymph node areaAxillary lymph nodes removed by traditional approach

### AND

Possible radiation to supraclavicular and/or internal mammary lymph nodes, IF axillary nodes are involved

### Local Treatment

to other parts of the body (applies only to metastatic disease) Does not apply

### Systemic Treatment

Hormonal (anti-estrogen) May be used for local and systemic benefits

### Systemic Treatment

ChemotherapyAlmost always recommended

### Systemic Treatment

Immune therapyAvailable only in clinical trials
Treatment OptionsSTAGE IIIB invasive (tumor extends to chest wall OR cancer involves breast skin or internal mammary lymph nodes)

### Local Treatment

to the breast areaModified radical mastectomy that may require removal of other nearby tissues involved with the tumor

### AND

Radiation before or after mastectomy

### Local Treatment

to the lymph node area Axillary lymph nodes removed by traditional approach AND

Possible radiation to supraclavicular and/or internal mammary lymph nodes, IF axillary nodes are involved

### Local Treatment

to other parts of the body (applies only to metastatic disease) Does not apply

### Systemic Treatment

Hormonal (anti-estrogen) May be used for local and systemic benefits

### Systemic Treatment

Chemotherapy Almost always recommended

### Systemic Treatment

Immune therapy Available only in clinical trials
Treatment OptionsSTAGE IV invasive; metastatic

### Local Treatment

to the breast areaSurgery, radiation, or both may be used, depending upon many individual factors

### Local Treatment

to the lymph node areaEnlarged lymph nodes may be treated if they are producing signs (medical findings) or uncomfortable symptoms

### Local Treatment

to other parts of the body (applies only to metastatic disease) Radiation most commonly used to relieve specific signs (medical findings) or uncomfortable symptoms

### AND/OR

Surgery may also have a role in dealing with specific signs or symptoms

### Systemic Treatment

Hormonal (anti-estrogen) May be used for local and systemic benefits

### Systemic Treatment

Chemotherapy Almost always recommended

### Systemic Treatment

Immune therapy Herceptin used IF the cancer tests HER2/neu positive

### Radiation Treatment Schedule:

For radiation to the breast and lymph node areas, you will receive treatment once a day, five days a week, for five to seven weeks. Partial-breast radiation is usually given twice a day for one week. For treatment to areas where the cancer has spread, daily treatments for two to three weeks are the norm.

### Radiation Types:

### External:

The most common type of radiation is known as external beam. In this technique, a large machine called a linear accelerator delivers high-energy radiation to the affected area. The linear accelerator creates high-energy radiation to treat cancers, using electricity to form a stream of fast-moving subatomic particles. You'll receive this form of radiation as an outpatient in daily sessions over five to seven weeks, depending on your particular situation.

### Internal:

Several types of radiation may be delivered from inside the body. These have several different names, including internal radiation, brachytherapy, low- or high-dose rate radiation, intracavitary radiation, and intraoperative radiation.
For internal radiation treatment, special substances are used that give off radiation. Very small pieces of these radioactive substances, called seeds, are used for cancer treatment. Some seeds give off radiation slowly (treatment is given over days). This is called low-dose rate or brachytherapy. Some seeds give off radiation quickly (treatment is given for 5-10 minutes). This is called high-dose rate radiation.

### Partial Breast Radiation:

The current standard of care is to treat the whole breast with radiation after lumpectomy. But another option is available: partial-breast radiation. It's also known as partial-breast irradiation (PBI) or limited-field radiation therapy. Researchers are studying partial-breast radiation to see how the benefits compare to whole-breast radiation. Partial-breast radiation was developed to reduce recurrence, shorten the length of time it takes to get radiation treatment, and limit the dose of radiation (and associated side effects) to surrounding normal tissue. Partial-breast radiation also MAY be able to be given again—but only to another part of the breast—if a new breast cancer is diagnosed in the future. Whole-breast radiation usually can't be given again to the same breast.

### Hormone Therapy:

Aromatase inhibitors are now considered the standard of care for post-menopausal women with hormone-receptor-positive breast cancer. Tamoxifen remains the hormonal treatment of choice for pre-menopausal women.
Clinical trials have shown the important benefits of aromatase inhibitors. Now medical experts consider aromatase inhibitors to be the new standard of care for post-menopausal women with invasive hormone-receptor-positive breast cancer, both early and advanced-stage.
The latest results of several major international trials showed that aromatase inhibitors work better than tamoxifen in post-menopausal women with early-stage breast cancer that is hormone-receptor-positive—estrogen-receptor-positive, progesterone-receptor-positive, or both.
Aromasin is a Type 1 “steroidal inhibitor,” which stops the activity of the aromatase enzyme forever.
Arimidex and Femara are both Type 2 “non-steroidal inhibitors.” They also stop the activity of the aromatase enzyme, but not permanently.

### For Pre-Menopausal Women:

SERMs block (or selectively inhibit) estrogen receptors in breast cells. Therefore, cells don't get the signals they need to grow and multiply.
SERMs stimulate estrogen receptors in other organs, with good and bad results. For example, the SERM tamoxifen:
   stimulates liver cells, lowering cholesterol levels
   stimulates bone cells, resulting in stronger bones and reduced risk of bone breaks
   stimulates growth of uterine cells (cells in the uterus), slightly increasing the risk of uterine Cancer
      A SERM may also weakly stop the formation of new blood vessels that supply the nutrients the cancer needs to grow. (This is called an “anti-angiogenic” effect.) Although this action would never be enough to stop making all new blood vessels, it may starve some cancer cells, which need extra blood vessels to grow.
      As long as a SERM is sitting inside all the estrogen receptors, the cancer cells remain quiet and relatively harmless. After a long period of not being stimulated, the cancer cells may die off. SERMs may even cause breast cancer cells to destroy themselves, a process called “apoptosis,” or programmed cell death.
      There are three SERMs, each usually taken once a day by pill:
The most prescribed SERM is tamoxifen (the brand name is Nolvadex, but it's also now a generic drug called tamoxifen citrate).
EVISTA® (chemical name: raloxifene) hasn't been used to treat women with breast cancer. But EVISTA® does lower the risk of breast cancer in post-menopausal women who take it to treat osteoporosis. EVISTA® also is as effective as tamoxifen in reducing the risk of breast cancer in post-menopausal women at increased risk but with no personal history of the disease.
The third SERM, Fareston (chemical name: toremifene), is relatively new and not often used in the United States.

### Prostate Cancer:

Definition of prostate cancer: Cancer that forms in tissues of the prostate (a gland in the male reproductive system found below the bladder and in front of the rectum). Prostate cancer usually occurs in older men.
Estimated new cases and deaths from prostate cancer in the United States in 2007:
New cases: 218,890
Deaths: 27,050

### Survival Rates:

Overall, 99% of men diagnosed with prostate cancer survive at least 5 years. Ninety one percent of all prostate cancers are found while they are still within the prostate or only in nearby areas. The 5-year relative survival rate for these men is nearly 100%. For the men whose cancer has already spread to distant parts of the body when it is found, about 32% will survive at least 5 years.

### Stages of Prostate Cancer:

### Stage I

In stage I, cancer is found in the prostate only. It cannot be felt during a digital rectal exam and is not visible by imaging. It is usually found accidentally during surgery for other reasons, such as benign prostatic hyperplasia. The Gleason score is low. Stage I prostate cancer may also be called stage A1 prostate cancer.

### Stage II

In stage II, cancer is more advanced than in stage I, but has not spread outside the prostate.
The Gleason score can range from 2-10. Stage II prostate cancer may also be called stage A2, stage B1, or stage B2 prostate cancer.

### Stage III

In stage III, cancer has spread beyond the outer layer of the prostate to nearby tissues. Cancer may be found in the seminal vesicles. The Gleason score can range from 2-10. Stage III prostate cancer may also be called stage C prostate cancer.

### Stage IV

In stage IV, cancer has metastasized (spread) to lymph nodes near or far from the prostate or to other parts of the body, such as the bladder, rectum, bones, liver, or lungs. Metastatic prostate cancer often spreads to the bones. The Gleason score can range from 2-10. Stage IV prostate cancer may also be called stage D1 or stage D2 prostate cancer.

### Treatments:

Watchful waiting
Watchful waiting is closely monitoring a patient's condition without giving any treatment until symptoms appear or change. This is usually used in older men with other medical problems and early-stage disease.

### Surgery

Patients in good health are usually offered surgery as treatment for prostate cancer. The following types of surgery are used:
Pelvic lymphadenectomy: A surgical procedure to remove the lymph nodes in the pelvis. A pathologist views the tissue under a microscope to look for cancer cells. If the lymph nodes contain cancer, the doctor will not remove the prostate and may recommend other treatment.
Radical prostatectomy: A surgical procedure to remove the prostate, surrounding tissue, and seminal vesicles. There are 2 types of radical prostatectomy:
   Retropubic prostatectomy: A surgical procedure to remove the prostate through an incision (cut) in the abdominal wall. Removal of nearby lymph nodes may be done at the same time.
   Perineal prostatectomy: A surgical procedure to remove the prostate through an incision (cut) made in the perineum (area between the scrotum and anus). Transurethral resection of the prostate (TURP): A surgical procedure to remove tissue from the prostate using a resectoscope (a thin, lighted tube with a cutting tool) inserted through the urethra. This procedure is sometimes done to relieve symptoms caused by a tumor before other cancer treatment is given. Transurethral resection of the prostate may also be done in men who cannot have a radical prostatectomy because of age or illnessNearby lymph nodes may also be removed through a separate incision in the abdomen.

### Radiation Therapy

Radiation therapy is a cancer treatment that uses high-energy x-rays or other types of radiation to kill cancer cells or keep them from growing. There are two types of radiation therapy. External radiation therapy uses a machine outside the body to send radiation toward the cancer. Internal radiation therapy uses a radioactive substance sealed in needles, seeds, wires, or catheters that are placed directly into or near the cancer. The way the radiation therapy is given depends on the type and stage of the cancer being treated. Impotence and urinary problems may occur in men treated with radiation therapy.
External Radiation is Employed More than Internal.
Long-term results with radiation therapy are dependent on stage. A retrospective review of 999 patients treated with megavoltage radiation therapy showed cause-specific survival rates to be significantly different at 10 years by T-stage: T1 (79%), T2 (66%), T3 (55%), and T4 (22%).[16] An initial serum prostate-specific antigen (PSA) level higher than 15 ng/mL is a predictor of probable failure with conventional radiation therapy.[17] Several randomized studies have demonstrated an improvement in freedom from biochemical (PSA-based) recurrence with higher doses of radiation therapy (78 Gy-79 Gy) as compared to conventional doses (68 Gy-70 Gy).[18-20][Level of evidence: liiDii] The higher doses were delivered using conformal techniques. None of the studies demonstrated a cause-specific survival benefit to higher doses; however, an ongoing study through the Radiation Therapy Oncology Group will be powered for OS.
Interstitial brachytherapy has been employed in several centers, generally for patients with T1 and T2 tumors. Patients are selected for favorable characteristics, including low Gleason score, low PSA level, and stage T1 to T2 tumors. Information and further study are required to better define the effects of modern interstitial brachytherapy on disease control and quality of life and to determine the contribution of favorable patient selection to outcomes.[21][Level of evidence: 3iiiDiii] Information about ongoing clinical trials is available from the NCI Web site.

### Hormone Therapy

Hormone therapy is a cancer treatment that removes hormones or blocks their action and stops cancer cells from growing. Hormones are substances produced by glands in the body and circulated in the bloodstream. In prostate cancer, male sex hormones can cause prostate cancer to grow. Drugs, surgery, or other hormones are used to reduce the production of male hormones or block them from working.
Hormone therapy used in the treatment of prostate cancer may include the following:
Luteinizing hormone-releasing hormone agonists can prevent the testicles from producing testosterone. Examples are leuprolide, goserelin, and buserelin.
Antiandrogens can block the action of androgens (hormones that promote male sex characteristics). Two examples are flutamide and nilutamide.
Drugs that can prevent the adrenal glands from making androgens include ketoconazole and aminoglutethimide.
Orchiectomy is a surgical procedure to remove one or both testicles, the main source of male hormones, to decrease hormone production.
Estrogens (hormones that promote female sex characteristics) can prevent the testicles from producing testosterone. However, estrogens are seldom used today in the treatment of prostate cancer because of the risk of serious side effects.

### Treatments by Stage:

### Stage I Prostate Cancer

Treatment of stage I prostate cancer may include the following:
Watchful waiting.
Radical prostatectomy, usually with pelvic lymphadenectomy, with or without radiation therapy after surgery. It may be possible to remove the prostate without damaging nerves that are necessary for an erection.
External-beam radiation therapy.
Implant radiation therapy.
A clinical trial of high-intensity focused ultrasound.
A clinical trial of radiation therapy.
A clinical trial testing new types of treatment.

### Stage II Prostate Cancer

Treatment of stage II prostate cancer may include the following:
Radical prostatectomy, usually with pelvic lymphadenectomy, with or without radiation therapy after surgery. It may be possible to remove the prostate without damaging nerves that are necessary for an erection.
Watchful waiting.
External-beam radiation therapy with or without hormone therapy.
Implant radiation therapy.
A clinical trial of radiation therapy with or without hormone therapy.
A clinical trial of ultrasound-guided cryosurgery.
A clinical trial of hormone therapy followed by radical prostatectomy.
A clinical trial testing new types of treatment.

### Stage III Prostate Cancer

Treatment of stage III prostate cancer may include the following:
External-beam radiation therapy with or without hormone therapy.
Hormone therapy.
Radical prostatectomy, usually with pelvic lymphadenectomy, with or without radiation therapy after surgery.
Watchful waiting.
Radiation therapy, hormone therapy, or transurethral resection of the prostate as palliative therapy to relieve symptoms caused by the cancer.
A clinical trial of radiation therapy.
A clinical trial of ultrasound-guided cryosurgery.
A clinical trial testing new types of treatment.

### Stage IV Prostate Cancer

Treatment of stage IV prostate cancer may include the following:
Hormone therapy.
External-beam radiation therapy with or without hormone therapy.
Radiation therapy or transurethral resection of the prostate as palliative therapy to relieve symptoms caused by the cancer.
Watchful waiting.
A clinical trial of radical prostatectomy with orchiectomy.

### Colon Cancer:

Definition of colon cancer: Cancer that forms in the tissues of the colon (the longest part of the large intestine). Most colon cancers are adenocarcinomas (cancers that begin in cells that make and release mucus and other fluids).
Estimated new cases and deaths from colon and rectal cancer in the United States in 2007:
New cases: 112,340 (colon)

### Colorectal Cancer Survival Rates

Nine out of 10 people whose colorectal cancer is found and treated at an early stage, before it has spread, live at least five years. Once the cancer has spread to nearby organs or lymph nodes, the 5-year survival rate goes down. The 5-year survival rate is the percentage of patients who are alive 5 years after their cancer is found (leaving out those who die of other causes). Of course, patients might live more than 5 years after their cancer is found.

Colon cancer survival rates*

| | |
|---|---|
| Stage I | 93% |
| Stage IIA | 85% |
| Stage IIB | 72% |
| Stage IIIA | 83% |
| Stage IIIB | 64% |
| Stage IIIC | 44% |
| Stage IV | 8% |

While combined modality therapy with chemotherapy and radiation therapy has a significant role in the management of patients with rectal cancer (below the peritoneal reflection), the role of adjuvant radiation therapy for patients with colon cancer (above the peritoneal reflection) is not well defined. Patterns-of-care analyses and single-institution retrospective reviews suggest a role for radiation therapy in certain high-risk subsets of colon cancer patients (T4, tumor location in immobile sites, local perforation, obstruction, and residual disease postresection).[37-42] Such observations led to the development of a phase III randomized intergroup study designed to test the benefit of adding radiation therapy to surgery and chemotherapy with 5-FU-levamisole for selected high-risk colon cancer patients (T4; or T3, N1 N2 ascending and/or descending colon).[43] This clinical trial closed early secondary to inadequate patient accrual, and analysis of 222 enrolled patients (the original goal was 700 patients) demonstrated no relapse or OS benefit for the group receiving radiation therapy, though the sample size and statistical power were inadequate to exclude benefit. Adjuvant radiation therapy, has no current standard role in the management of patients with colon cancer following curative resection, though it may have a role for patients with residual disease. Radiation therapy is seldom used to treat metastatic colon cancer because of side effects and relative resistance when given at the lower tolerated doses.

### Stages:

### Stage 0 (Carcinoma in Situ)

In stage 0, abnormal cells are found in the innermost lining of the colon. These abnormal cells may become cancer and spread into nearby normal tissue. Stage 0 is also called carcinoma in situ.

### Stage I

In stage I, cancer has formed and spread beyond the innermost tissue layer of the colon wall to the middle layers. Stage I colon cancer is sometimes called Dukes A colon cancer.

### Stage II

Stage II colon cancer is divided into stage IIA and stage IIB.
Stage IIA: Cancer has spread beyond the middle tissue layers of the colon wall or has spread to nearby tissues around the colon or rectum.
Stage IIB: Cancer has spread beyond the colon wall into nearby organs and/or through the peritoneum.
   Stage II colon cancer is sometimes called Dukes B colon cancer.

### Stage III

Stage III colon cancer is divided into stage IIIA, stage IIIB, and stage IIIC.
Stage IIIA: Cancer has spread from the innermost tissue layer of the colon wall to the middle layers and has spread to as many as 3 lymph nodes.
Stage IIIB: Cancer has spread to as many as 3 nearby lymph nodes and has spread:
   beyond the middle tissue layers of the colon wall; or
   to nearby tissues around the colon or rectum; or
   beyond the colon wall into nearby organs and/or through the peritoneum.
Stage IIIC: Cancer has spread to 4 or more nearby lymph nodes and has spread:
   to or beyond the middle tissue layers of the colon wall; or
   to nearby tissues around the colon or rectum; or
   to nearby organs and/or through the peritoneum.
      Stage III colon cancer is sometimes called Dukes C colon cancer.

### Stage IV

In stage IV, cancer may have spread to nearby lymph nodes and has spread to other parts of the body, such as the liver or lungs. Stage IV colon cancer is sometimes called Dukes D colon cancer.

### Treatments by Stage:

### Stage 0 (Carcinoma in Situ)

Treatment of stage 0 (carcinoma in situ) may include the following types of surgery:
Local excision or simple polypectomy.
Resection/anastomosis. This is done when the tumor is too large to remove by local excision.
   Check for clinical trials from NCI's PDQ Cancer Clinical Trials Registry that are now accepting patients with stage 0 colon cancer.

### Stage I Colon Cancer

Treatment of stage I colon cancer is usually resection/anastomosis.
Check for clinical trials from NCI's PDQ Cancer Clinical Trials Registry that are now accepting patients with stage I colon cancer.

### Stage II Colon Cancer

Treatment of stage II colon cancer may include the following:
Resection/anastomosis.
Clinical trials of chemotherapy, radiation therapy, or biologic therapy after surgery.
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with stage II colon cancer.

### Stage III Colon Cancer

Treatment of stage III colon cancer may include the following:
Resection/anastomosis with chemotherapy.
Clinical trials of chemotherapy, radiation therapy, and/or biologic therapy after surgery.
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with stage III colon cancer.

### Stage IV and Recurrent Colon Cancer

Treatment of stage IV and recurrent colon cancer may include the following:
Resection/anastomosis (surgery to remove the cancer or bypass the tumor and join the cut ends of the colon).
Surgery to remove parts of other organs, such as the liver, lungs, and ovaries, where the cancer may have recurred or spread.
Radiation therapy or chemotherapy may be offered to some patients as palliative therapy to relieve symptoms and improve quality of life.
Clinical trials of chemotherapy and/or biologic therapy.
   Treatment of locally recurrent colon cancer may be local excision.
   Special treatments of cancer that has spread to or recurred in the liver may include the following:
Chemotherapy followed by resection.
Radiofrequency ablation or cryosurgery.
Clinical trials of hepatic chemoembolization with radiation therapy.
   Patients whose colon cancer spreads or recurs after initial treatment with chemotherapy may be offered further chemotherapy with a different drug or combination of drugs.

### Rectal Cancer:

Definition of rectal cancer: Cancer that forms in the tissues of the rectum (the last several inches of the large intestine before the anus).
New Cases of rectal cancer in 2007 41,420 Deaths: 52,180 (colon and rectal combined)

### Survival:

Cancer of the rectum is a highly treatable and often curable disease when localized. Surgery is the primary treatment and results in cure in approximately 45% of all patients.

### Stages:

### Stage 0 (Carcinoma In Situ)

In stage 0, abnormal cells are found in the innermost lining of the rectum. These abnormal cells may become cancer and spread into nearby normal tissue. Stage 0 is also called carcinoma in situ.

### Stage I

In stage I, cancer has formed and spread beyond the innermost lining of the rectum to the second and third layers and involves the inside wall of the rectum, but it has not spread to the outer wall of the rectum or outside the rectum. Stage I rectal cancer is sometimes called Dukes A rectal cancer.

### Stage II

In stage II, cancer has spread outside the rectum to nearby tissue, but it has not gone into the lymph nodes (small, bean-shaped structures found throughout the body that filter substances in a fluid called lymph and help fight infection and disease). Stage II rectal cancer is sometimes called Dukes B rectal cancer.

### Stage III

In stage III, cancer has spread to nearby lymph nodes, but it has not spread to other parts of the body. Stage III rectal cancer is sometimes called Dukes C rectal cancer.

### Stage IV

In stage IV, cancer has spread to other parts of the body, such as the liver, lungs, or ovaries. Stage IV rectal cancer is sometimes called Dukes D rectal cancer.

### Treatment Options by Stage

### Stage 0 (Carcinoma In Situ)

Treatment of stage 0 may include the following:
Local excision (surgery to remove the tumor without cutting into the abdomen) or simple polypectomy (surgery to remove a growth that protrudes from the rectal mucous membrane).
Resection (surgery to remove the tumor). This is done when the tumor is too large to remove by local excision.
Internal or external radiation therapy.
   Check for clinical trials from NCI's PDQ Cancer Clinical Trials Registry that are now accepting patients with stage 0 rectal cancer.

### Stage I Rectal Cancer

Treatment of stage I rectal cancer may include the following:
Surgery to remove the tumor with or without anastomosis (joining the cut ends of the rectum).
Surgery to remove the tumor with or without radiation therapy and chemotherapy.
Internal and/or external radiation therapy.
   Check for clinical trials from NCI's PDQ Cancer Clinical Trials Registry that are now accepting patients with stage I rectal cancer.

### Stage II Rectal Cancer

Treatment of stage II rectal cancer may include the following:
Resection with or without anastomosis (joining the cut ends of the rectum and colon, or the colon and anus) followed by chemotherapy and radiation therapy.
Partial or total pelvic exenteration (surgery to remove the organs and nearby structures of the pelvis), depending on where the cancer has spread. Surgery is followed by radiation therapy and chemotherapy.
Radiation therapy with or without chemotherapy followed by surgery and chemotherapy.
Radiation therapy during surgery followed by external-beam radiation therapy and chemotherapy.
A clinical trial evaluating new treatment options.
   Information about ongoing clinical trials is available from the NCI Web site.
   Check for clinical trials from NCI's PDQ Cancer Clinical Trials Registry that are now accepting patients with stage II rectal cancer.

### Stage III Rectal Cancer

Treatment of stage III rectal cancer may include the following:
Resection with or without anastomosis (joining the cut ends of the rectum and colon, or the colon and anus) followed by chemotherapy and radiation therapy.
Partial or total pelvic exenteration (surgery to remove the organs and nearby structures of the pelvis), depending on where the cancer has spread. Surgery is followed by radiation therapy and chemotherapy.
Radiation therapy with or without chemotherapy followed by surgery and chemotherapy.
Radiation therapy during surgery followed by external-beam radiation therapy and chemotherapy.
Chemotherapy and radiation therapy to relieve symptoms caused by advanced cancer.
A clinical trial evaluating new treatment options.
   Information about ongoing clinical trials is available from the NCI Web site. Check for clinical trials from NCI's PDQ Cancer Clinical Trials Registry that are now accepting patients with stage III rectal cancer.

### Stage IV Rectal Cancer

Treatment of stage IV rectal cancer may include the following:
Resection/anastomosis (surgery to remove the cancer and join the cut ends of the rectum and colon, or colon and anus) to relieve symptoms caused by advanced cancer.
Surgery to remove parts of other organs, such as the liver, lung, and ovaries, where the cancer may have spread.
Chemotherapy and radiation therapy to relieve symptoms caused by advanced cancer.
Chemotherapy following surgery.
Clinical trials of chemotherapy and biological therapy.

### Bladder Cancer:

Definition of bladder cancer: Cancer that forms in tissues of the bladder (the organ that stores urine). Most bladder cancers are transitional cell carcinomas (cancer that begins in cells that normally make up the inner lining of the bladder). Other types include squamous cell carcinoma (cancer that begins in thin, flat cells) and adenocarcinoma (cancer that begins in cells that make and release mucus and other fluids). The cells that form squamous cell carcinoma and adenocarcinoma develop in the inner lining of the bladder as a result of chronic irritation and inflammation.
Estimated new cases and deaths from bladder cancer in the United States in 2007:
New cases: 67,160
Deaths: 13,750

### Survival:

The survival rate for bladder cancer is considered very good. ACS says if it's discovered early, before it spreads, the five year survival rate is 94 percent. When the cancer has spread to the organs in the pelvic region, the rate drops to 49 percent and once it has spread to other organs the survival rate falls to 6 percent.
Patients with invasive bladder cancer may benefit from getting chemotherapy before surgery or radiation therapy, according to a new review of previously published studies. Researchers from the Advanced Bladder Cancer Meta-analysis Collaboration, based in London, looked at the combined results from 10 clinical trials to assess whether or not chemotherapy given before local treatment (known as neoadjuvant chemotherapy) had an effect on outcome.
Examining data from more than 2,600 patients, the researchers found that combination chemotherapy (using more than one drug) improved five-year survival by 5%. They reported their findings in The Lancet (Vol. 361, No. 9373:1927-1934).

### Treatments by Stage:

In North America, the standard treatment of patients with invasive bladder cancers is radical cystectomy and urinary diversion. Other treatment approaches include TUR and segmental resection with or without radiation therapy, combined chemotherapy-radiation therapy, or either followed by salvage cystectomy, when needed, for local failure.

### Stage 0 (Papillary Carcinoma and Carcinoma In Situ)

Treatment of stage 0 may include the following:
Transurethral resection with fulguration.
Transurethral resection with fulguration followed by intravesical biologic therapy or chemotherapy.
Segmental cystectomy.
Radical cystectomy.
A clinical trial of photodynamic therapy.
A clinical trial of biologic therapy.
A clinical trial of chemoprevention therapy given after treatment so the condition will not recur (come back).
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with stage 0 bladder cancer.

### Stage I Bladder Cancer

Treatment of stage I bladder cancer may include the following:
Transurethral resection with fulguration.
Transurethral resection with fulguration followed by intravesical biologic therapy or chemotherapy.
Segmental or radical cystectomy.
Radiation implants with or without external radiation therapy.
A clinical trial of chemoprevention therapy given after treatment to stop cancer from recurring (coming back).
A clinical trial of intravesical therapy.
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with stage I bladder cancer.

### Stage II Bladder Cancer

Treatment of stage II bladder cancer may include the following:
Radical cystectomy with or without surgery to remove pelvic lymph nodes.
Combination chemotherapy followed by radical cystectomy.
External radiation therapy combined with chemotherapy.
Radiation implants before or after external radiation therapy.
Transurethral resection with fulguration.
Segmental cystectomy.
   Check for clinical trials from NCI's PDQ Cancer Clinical Trials Registry that are now accepting patients with stage II bladder cancer.

### Stage III Bladder Cancer

Treatment of stage III bladder cancer may include the following:
Radical cystectomy with or without surgery to remove pelvic lymph nodes.
Combination chemotherapy followed by radical cystectomy.
External radiation therapy combined with chemotherapy.
External radiation therapy with radiation implants.
Segmental cystectomy.
   Check for clinical trials from NCI's PDQ Cancer Clinical Trials Registry that are now accepting patients with stage III bladder cancer.

### Stage IV Bladder Cancer

Treatment of stage IV bladder cancer may include the following:
Radical cystectomy with surgery to remove pelvic lymph nodes.
External radiation therapy (may be as palliative therapy to relieve symptoms and improve quality of life).
Urinary diversion as palliative therapy to relieve symptoms and improve quality of life.
Cystectomy as palliative therapy to relieve symptoms and improve quality of life.
Chemotherapy alone or after local treatment (surgery or radiation therapy).
A clinical trial of chemotherapy.

### Lung Cancer:

### Definition:

Cancer that forms in tissues of the lung, usually in the cells lining air passages. The two main types are small cell lung cancer and non-small cell lung cancer. These types are diagnosed based on how the cells look under a microscope.
Small-Cell Lung Cancer Differs from Non-Small Cell Lung Cancer in the Following Ways:
Small-cell lung cancer grows rapidly.
Small-cell lung cancer spreads quickly.
Small-cell lung cancer responds well to chemotherapy (using medications to kill cancer cells) and radiation therapy (using high-dose x-rays or other high-energy rays to kill cancer cells).
Small-cell lung cancer is frequently associated with distinct paraneoplastic syndromes (collection of symptoms that result from substances produced by the tumor, occurring far away from the tumor).
   Estimated new cases and deaths from lung cancer (non-small cell and small cell combined) in the United States in 2007:
New cases: 213,380
Deaths: 160,390

### Non-Small Cell Lung Cancer

Non-small cell lung cancer is a disease in which malignant (cancer) cells form in the tissues of the lung.

### Stage 5-Year Relative Survival Rate

### I 47%

### II 26%

### III 8%

### IV 2%

### There are Several Types of Non-Small Cell Lung Cancer: 75% of all Lung Cancers are Non-Small Cell Lung Cancer

Each type of non-small cell lung cancer has different kinds of cancer cells. The cancer cells of each type grow and spread in different ways. The types of non-small cell lung cancer are named for the kinds of cells found in the cancer and how the cells look under a microscope:
Squamous cell carcinoma: Cancer that begins in squamous cells, which are thin, flat cells that look like fish scales. This is also called epidermoid carcinoma.
Large cell carcinoma: Cancer that may begin in several types of large cells.
Adenocarcinoma: Cancer that begins in the cells that line the alveoli and make substances such as mucus.

### The Following Stages are Used for Non-Small Cell Lung Cancer:

### Occult (Hidden) Stage

In the occult (hidden) stage, cancer cells are found in sputum (mucus coughed up from the lungs), but no tumor can be found in the lung by imaging or bronchoscopy, or the primary tumor is too small to be checked.

### Stage 0 (Carcinoma in Situ)

In stage 0, abnormal cells are found in the innermost lining of the lung. These abnormal cells may become cancer and spread into nearby normal tissue. Stage 0 is also called carcinoma in situ.

### Stage I

In stage I, cancer has formed. Stage I is divided into stages IA and IB:
Stage IA: The tumor is in the lung only and is 3 centimeters or, smaller.
Stage IB: One or more of the following is true:
   The tumor is larger than 3 centimeters.
   Cancer has spread to the main bronchus of the lung, and is at least 2 centimeters from the carina (where the trachea joins the bronchi).
   Cancer has spread to the innermost layer of the membrane that covers the lungs.
   The tumor partly blocks the bronchus or bronchioles and part of the lung has collapsed or developed pneumonitis (inflammation of the lung).

### Stage II

Stage IIA: The tumor is 3 centimeters or smaller and cancer has spread to nearby lymph nodes on the same side of the chest as the tumor.
Stage IIB:
   Cancer has spread to nearby lymph nodes on the same side of the chest as the tumor and one or more of the following is true:
      The tumor is larger than 3 centimeters.
      Cancer has spread to the main bronchus of the lung and is 2 centimeters or more from the carina (where the trachea joins the bronchi).
      Cancer has spread to the innermost layer of the membrane that covers the lungs.
      The tumor partly blocks the bronchus or bronchioles and part of the lung has collapsed or developed pneumonitis (inflammation of the lung).
   or
   Cancer has not spread to lymph nodes and one or more of the following is true:
      The tumor may be any size and cancer has spread to the chest wall, or the diaphragm, or the pleura between the lungs, or membranes surrounding the heart.
      Cancer has spread to the main bronchus of the lung and is no more than 2 centimeters from the carina (where the trachea meets the bronchi), but has not spread to the trachea.
      Cancer blocks the bronchus or bronchioles and the whole lung has collapsed or developed pneumonitis (inflammation of the lung).

### Stage IIIA

In stage IIIA, cancer has spread to lymph nodes on the same side of the chest as the tumor.

### Also:

The tumor may be any size.
Cancer may have spread to the main bronchus, the chest wall, the diaphragm, the pleura around the lungs, or the membrane around the heart, but has not spread to the trachea.
Part or all of the lung may have collapsed or developed pneumonitis (inflammation of the lung).

### Stage IIIB

In stage IIIB, the tumor may be any size and has spread:
To lymph nodes above the collarbone or in the opposite side of the chest from the tumor; and/or
To any of the following:
   Heart.
   Major blood vessels that lead to or from the heart.
   Chest wall.
   Diaphragm.
   Trachea.
   Esophagus.
   Sternum (chest bone) or backbone.
   More than one place in the same lobe of the lung.
   The fluid of the pleural cavity surrounding the lung.

### Stage IV

In stage IV, cancer may have spread to lymph nodes and has spread to another lobe of the lungs or to other parts of the body, such as the brain, liver, adrenal glands, kidneys, or bone.

### Treatments by Stage:

### Stage 0 (Carcinoma In Situ)

Treatment of stage 0 may include the following:
Surgery (wedge resection or segmental resection).
Photodynamic therapy using an endoscope.

### Stage I Non-Small Cell Lung Cancer

Treatment of stage I non-small cell lung cancer may include the following:
Surgery (wedge resection, segmental resection, or lobectomy).
External radiation therapy (for patients who cannot have surgery or choose not to have surgery).
Surgery followed by chemotherapy.
A clinical trial of photodynamic therapy using an endoscope.
A clinical trial of surgery followed by chemoprevention.

### Stage II Non-Small Cell Lung Cancer

Treatment of stage II non-small cell lung cancer may include the following:
Surgery (wedge resection, segmental resection, lobectomy, or pneumonectomy).
External radiation therapy (for patients who cannot have surgery or choose not to have surgery).
Surgery followed by chemotherapy, with or without other treatments.
A clinical trial of external radiation therapy following surgery.

### Stage IIIA and Stage IIIB Non-Small Cell Lung Cancer

Treatment of stage IIIA non-small cell lung cancer may include the following:
Surgery with or without radiation therapy.
External radiation therapy alone.
Chemotherapy combined with other treatments.
A clinical trial of new ways of giving radiation therapy and chemotherapy.
A clinical trial of new combinations of treatments.
   Treatment of stage IIIB non-small cell lung cancer may include the following:
External radiation therapy alone.
Chemotherapy combined with external radiation therapy.
Chemotherapy combined with external radiation therapy, followed by surgery.
Chemotherapy alone.
A clinical trial of new ways of giving radiation therapy.
A clinical trial of new combinations of treatments.

### Stage IV Non-Small Cell Lung Cancer

Treatment of stage IV non-small cell lung cancer may include the following:
Watchful waiting.
External radiation therapy as palliative therapy, to relieve pain and other symptoms and improve the quality of life.
Chemotherapy.
Laser therapy and/or internal radiation therapy.
A clinical trial of chemotherapy with or without biologic therapy.

### Small Cell Lung Cancer 25% of all Lung Cancers

### Treatment by Stages:

### Limited-Stage Small Cell Lung Cancer

Treatment of limited-stage small cell lung cancer may include the following:
Combination chemotherapy and radiation therapy to the chest, with or without radiation therapy to the brain.
Combination chemotherapy with or without radiation therapy to the brain in patients with complete response.
Combination chemotherapy with or without radiation therapy to the chest.
Surgery followed by chemotherapy or chemotherapy plus radiation therapy to the chest, with or without radiation therapy to the brain.
Clinical trials of new chemotherapy, surgery, and radiation treatments.

### Extensive-Stage Small Cell Lung Cancer

Treatment of extensive-stage small cell lung cancer may include the following:
Chemotherapy.
Combination chemotherapy.
Combination chemotherapy with or without radiation therapy to the brain for patients with complete response.
Radiation therapy to the brain, spine, bone, or other parts of the body where the cancer has spread, as palliative therapy to relieve symptoms and improve quality of life.
Clinical trials of new chemotherapy treatments.

### Treatment Options for Recurrent Small Cell Lung Cancer

Treatment of recurrent small cell lung cancer may include the following:
Radiation therapy as palliative therapy to relieve symptoms and improve quality of life.
Chemotherapy as palliative therapy to relieve symptoms and improve quality of life.
Laser therapy, surgical placement of devices to keep the airways open, and/or internal radiation therapy, as palliative therapy to relieve symptoms and improve quality of life.
Clinical trials of chemotherapy.

### Chondrosarcoma:

### Definition/General Info

Chondrosarcoma is the second most frequent primary malignant tumor of bone, representing approximately 25% of all primary osseous neoplasms. Chondrosarcomas are a group of tumors with highly diverse features and behavior patterns, ranging from slow-growing non-metastasizing lesions to highly aggressive metastasizing sarcomas.

### Frequency:

The incidence rate of chondrosarcoma is dependent on patient age, peaking at 8 cases per 1 million population in those aged 80-84 years. The incidence in children is low. Most tumors arise in patients older than 40 years. The risk of chondrosarcoma is increased in people with enchondromatosis syndromes (eg, Oilier disease, Maffucci syndrome, metachondromatosis) and in those with hereditary multiple exostosis (eg, diaphyseal aclasis). Patients with these conditions are generally younger than other patients at presentation.

### Survival:

The 5-year survival rate for grade 1 lesions is 90%, and the rate decreases to 29% for grade 3 tumors. Grade 1 lesions do not metastasize. Metastatic spread, typically pulmonary, is more frequently associated with grade 3 lesions than with others. Lymph node spread is more common than with other osseous neoplasms.
Tumor recurrence typically occurs 5-10 years after surgery, and it is often associated with more aggressive behavior and a histologic grade higher than that of the original lesion.

### Treatment:

Treatment of chondrosarcoma is wide surgical excision. There is a very limited role for chemotherapy or radiation. Biopsies must be planned with future tumor excision in mind. Patients with adequately resected low grade chondrosarcomas have an excellent survival rate. The survival of patients with high grade tumors depends on the location, size and stage of the tumor.

### Lymphomas:

### Non-Hodgkin Lymphomas

NHL. Any of a large group of cancers of the immune system. NHLs can occur at any age and are often marked by enlarged lymph nodes, fever, and weight loss. There are many different types of NHL, which can be divided into aggressive (fast-growing) and indolent (slow-growing) types and can be classified as either B-cell or T-cell NHL. B-cell NHLs include Burkitt lymphoma, diffuse large B-cell lymphoma, follicular lymphoma, immunoblastic large cell lymphoma, precursor B-lymphoblastic lymphoma, and mantle cell lymphoma. T-cell NHLs include mycosis fungoides, anaplastic large cell lymphoma, and precursor T-lymphoblastic lymphoma. Lymphomas related to lymphoproliferative disorders following bone marrow or stem cell transplantation are usually B-cell NHLs. Prognosis and treatment depend on the stage and type of disease.
Estimated new cases and deaths from non-Hodgkin lymphoma in the United States in 2007:

### New Cases: 63,190

### Deaths: 18,660

### Adult Non-Hodgkins

Survival rates vary by stage and progression. The 5 year survival rate for an adult in the Stages I and II is 60-70%.

### Treatments by Stage:

### Indolent, Stage I and Contiguous Stage II Adult Non-Hodgkin Lymphoma

Treatment of indolent, stage I and contiguous stage II adult non-Hodgkin lymphoma may include the following:
Radiation therapy directed at the area where cancer is found.
Watchful waiting.
Radiation therapy directed at the area where cancer is found and nearby lymph nodes.
Chemotherapy with radiation therapy.

### Aggressive, Stage I and Contiguous Stage II Adult Non-Hodgkin Lymphoma

Treatment of aggressive, stage I and contiguous stage II adult non-Hodgkin lymphoma may include the following:
Combination chemotherapy with or without radiation therapy to areas where cancer is found.
A clinical trial of monoclonal antibody therapy and combination chemotherapy with steroids. Radiation therapy may also be given.
   Check for clinical trials from NCI's PDQ Cancer Clinical Trials Registry that are now accepting patients with aggressive, stage I adult non-Hodgkin lymphoma and aggressive, contiguous stage II adult non-Hodgkin lymphoma.

### Indolent, Noncontiguous Stage II/III/IV Adult Non-Hodgkin Lymphoma

Treatment of indolent, noncontiguous stage II/III/IV adult non-Hodgkin lymphoma may include the following:
Watchful waiting for patients who do not have symptoms.
Chemotherapy with or without steroids.
Combination chemotherapy with steroids.
Monoclonal antibody therapy with or without combination chemotherapy.
Radiolabeled monoclonal antibody therapy.
Radiation therapy directed at the area where cancer is found and nearby lymph nodes, for patients who have stage III disease.
A clinical trial of chemotherapy with or without total-body irradiation (radiation therapy to the entire body) or radiolabeled monoclonal antibody therapy, followed by autologous or allogeneic stem cell transplant.
A clinical trial of chemotherapy with or without vaccine therapy.
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with indolent, noncontiguous stage II adult non-Hodgkin lymphoma, indolent, stage III adult non-Hodgkin lymphoma and indolent, stage IV adult non-Hodgkin lymphoma.
   Aggressive, Noncontiguous Stage II/III/IV Adult Non-Hodgkin Lymphoma Treatment of aggressive, noncontiguous stage II/III/IV adult non-Hodgkin lymphoma may include the following:
Combination chemotherapy alone.
Combination chemotherapy with radiation therapy or monoclonal antibody therapy.
Combination chemotherapy with CNS prophylaxis.
A clinical trial of autologous or allogeneic stem cell transplant for patients who are likely to relapse.

### Adult Lymphoblastic Lymphoma

Treatment of adult lymphoblastic lymphoma may include the following:
Combination chemotherapy and CNS prophylaxis.
A clinical trial of autologous or allogeneic stem cell transplant.

### Diffuse Small Noncleaved Cell/Burkitt Lymphoma

Treatment of adult diffuse small noncleaved cell/Burkitt lymphoma may include the following:
Combination chemotherapy and CNS prophylaxis.
A clinical trial of combination chemotherapy.
A clinical trial of autologous or allogeneic stem cell transplant.
   This summary section refers to specific treatments under study in clinical trials, but it may not mention every new treatment being studied. Information about ongoing clinical trials is available from the NCI Web site.
   Check for clinical trials from NCI's PDQ Cancer Clinical Trials Registry that are now accepting patients with adult Burkitt lymphoma.

### Non-Hodgkin Lymphoma During Pregnancy

### Aggressive Non-Hodgkin Lymphoma During the First Trimester of Pregnancy

When aggressive non-Hodgkin lymphoma is diagnosed in the first trimester of pregnancy, medical oncologists may advise the patient to end her pregnancy so that treatment may begin. Treatment is usually chemotherapy with or without radiation therapy.

### Aggressive Non-Hodgkin Lymphoma During the Second and Third Trimester of Pregnancy

When possible, treatment should be postponed until after an early delivery, so that the fetus will not be exposed to anticancer drugs or radiation therapy. However, sometimes the cancer will need to be treated immediately in order to increase the mother's chance of survival.

### Indolent Non-Hodgkin Lymphoma During Pregnancy

Women who have indolent (slow-growing) non-Hodgkin lymphoma can usually delay treatment with watchful waiting.

### Recurrent Adult Non-Hodgkin Lymphoma

### Indolent, Recurrent Adult Non-Hodgkin Lymphoma

Treatment of indolent, recurrent adult non-Hodgkin lymphoma may include the following:
Chemotherapy with one or more drugs.
Radiation therapy.
Radiation therapy and/or chemotherapy as palliative therapy to relieve symptoms and improve quality of life.
Monoclonal antibody therapy.
A clinical trial of radiolabeled monoclonal antibody therapy.
A clinical trial of monoclonal antibody therapy as palliative therapy to relieve symptoms and improve quality of life.
A clinical trial of autologous or allogeneic stem cell transplant.
   Treatment of indolent lymphoma that comes back as aggressive lymphoma may include the following:
A clinical trial of autologous or allogeneic stem cell transplant.
A clinical trial of combination chemotherapy followed by radiation therapy or stem cell transplant and radiation therapy.
A clinical trial of monoclonal antibody therapy.
A clinical trial of radiolabeled monoclonal antibody therapy.

### Aggressive, Recurrent Adult Non-Hodgkin Lymphoma

Treatment of aggressive, recurrent adult non-Hodgkin lymphoma may include the following:
Stem cell transplant.
Monoclonal antibody therapy.
A clinical trial of autologous or allogeneic stem cell transplant.
A clinical trial of combination chemotherapy followed by radiation therapy or stem cell transplant and radiation therapy.
A clinical trial of radiolabeled monoclonal antibody therapy.
   Treatment of aggressive lymphoma that comes back as indolent lymphoma may include the following:
Chemotherapy.
Palliative therapy to relieve symptoms and improve quality of life.

### Childhood Non-Hodgkin Lymphoma

There are four major types of childhood non-Hodgkin lymphoma.
The specific type of lymphoma is determined by how the cells look under a microscope.
The 4 major types of childhood non-Hodgkin lymphoma are:
B-cell non-Hodgkin lymphoma (Burkitt and Burkitt-like lymphoma) and Burkitt leukemia.
Diffuse large B-cell lymphoma.
Lymphoblastic lymphoma.
Anaplastic large cell lymphoma.

### About 4.5% of Childhood Cancers are Non-Hodgkin Lymphomas.

### Survival Rate:

The 5-year survival rate for children and adolescents younger than age 20 with non-Hodgkin lymphoma ranges from around 70% for anaplastic lymphoma to around 85% to 90% for the others.

### Treatments:

Three types of standard treatment are used:

### Chemotherapy

Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column (intrathecal chemotherapy), an organ, or a body cavity such as the abdomen, the drugs mainly affect cancer cells in those areas. Intrathecal chemotherapy may be used to treat childhood non-Hodgkin lymphoma that has spread, or may spread, to the brain. When used to prevent spread to the brain, it is called CNS prophylaxis. The way the chemotherapy is given depends on the type and stage of the cancer being treated.
Combination chemotherapy is treatment using 2 or more anticancer drugs.
Radiation therapy (in certain patients) Radiation is not routinely used in Childhood Non-Hodgkin Lymphoma
Radiation therapy is a cancer treatment that uses high energy x-rays or other types of radiation to kill cancer cells or keep them from growing. There are two types of radiation therapy. External radiation therapy uses a machine outside the body to send radiation toward the cancer. Internal radiation therapy uses a radioactive substance sealed in needles, seeds, wires, or catheters that are placed directly into or near the cancer. When radiation therapy is used to prevent spread to the brain, it is called CNS prophylaxis. The way the radiation therapy is given depends on the type and stage of the cancer being treated.
High-Dose Chemotherapy with Stem Cell Transplant
This treatment is a way of giving high doses of chemotherapy and then replacing blood-forming cells destroyed by the cancer treatment. Stem cells (immature blood cells) are removed from the bone marrow or blood of the patient or a donor and are frozen and stored.
After the chemotherapy is completed, the stored stem cells are thawed and given back to the patient through an infusion. These reinfused stem cells grow into (and restore) the body's blood cells.

### Hodgkin Lymphoma Adult:

Definition of Hodgkin lymphoma: A cancer of the immune system that is marked by the presence of a type of cell called the Reed-Sternberg cell. The two major types of Hodgkin lymphoma are classical Hodgkin lymphoma and nodular lymphocyte-predominant Hodgkin lymphoma. Symptoms include the painless enlargement of lymph nodes, spleen, or other immune tissue. Other symptoms include fever, weight loss, fatigue, or night sweats. Also called Hodgkin disease.
Estimated new cases and deaths from Hodgkin lymphoma in the United States in 2007:

### New Cases: 8,190

### Deaths: 1,070

### Treatment by Stage:

### Early Favorable Hodgkin Lymphoma

Treatment of early favorable Hodgkin lymphoma may include the following:
Combination chemotherapy with or without radiation therapy to parts of the body with cancer.
Radiation therapy alone to areas of the body with cancer or to the mantle field (neck, chest, armpits).
Clinical trials of new combinations of chemotherapy and/or radiation therapy.

### Early Unfavorable Hodgkin Lymphoma

Treatment of early unfavorable Hodgkin lymphoma may include the following:
Combination chemotherapy with or without radiation therapy to parts of the body with cancer.
Clinical trials of new combinations of chemotherapy and/or radiation therapy.

### Advanced Favorable Hodgkin Lymphoma

Treatment of advanced favorable Hodgkin lymphoma may include the following:
Combination chemotherapy with or without radiation therapy to parts of the body with cancer.
Clinical trials of new combinations of chemotherapy.

### Advanced Unfavorable Hodgkin Lymphoma

Treatment of advanced unfavorable Hodgkin lymphoma may include the following:
Combination chemotherapy.
Clinical trials of new combinations of chemotherapy.
A clinical trial of high-dose chemotherapy and stem cell transplant using the patient's own stem cells.

### Hodgkin Lymphoma During Pregnancy

### Hodgkin Lymphoma During the First Trimester of Pregnancy

When Hodgkin lymphoma is diagnosed in the first trimester of pregnancy, it does not necessarily mean that the patient will be advised to end the pregnancy. Each patient's treatment will depend on the stage of the lymphoma, how fast it is growing, and the patient's wishes. For women who choose to continue the pregnancy, treatment of Hodgkin lymphoma during the first trimester of pregnancy may include the following:
Watchful waiting when the cancer is above the diaphragm and is slow-growing. Delivery may be induced when the fetus is 32 to 36 weeks old so the mother can begin treatment.
Radiation therapy above the diaphragm, with the fetus shielded.
Systemic chemotherapy using one or more drugs.

### Hodgkin Lymphoma During the Second Half of Pregnancy

When Hodgkin lymphoma is diagnosed in the second half of pregnancy, most patients can delay treatment until after the baby is born. Treatment of Hodgkin lymphoma during the second half of pregnancy may include the following:
Watchful waiting, with plans to induce delivery when the fetus is 32 to 36 weeks old.
Systemic chemotherapy using one or more drugs.
Steroid therapy
Radiation therapy to relieve breathing problems caused by a large tumor in the chest.

### Recurrent Adult Hodgkin Lymphoma

Treatment of recurrent Hodgkin lymphoma may include the following:
Combination chemotherapy.
Combination chemotherapy followed by high-dose chemotherapy and stem cell transplant with or without radiation therapy.
Radiation therapy with or without chemotherapy.
Chemotherapy as palliative therapy to relieve symptoms and improve quality of life.
A clinical trial of high-dose chemotherapy and stem cell transplant.

### Childhood Hodgkin Lymphoma:

Survival: About 90% of children with Hodgkin's disease go into remission (where there is no longer evidence of cancer cells in the body) following initial chemotherapy. A long-term cure (5 years disease-free or longer) is achieved in almost all Stage I or Stage II patients, in up to 90% of Stage III patients, and more than 60% of those with Stage IV.

### Treatment by Stages:

### Low-Risk Childhood Hodgkin Lymphoma

Treatment of low-risk childhood Hodgkin lymphoma may include the following:
Combination chemotherapy with low-dose radiation therapy to involved areas.
A clinical trial of combination chemotherapy with or without low-dose radiation therapy to involved areas.

### Intermediate-Risk Childhood Hodgkin Lymphoma

Treatment of intermediate-risk childhood Hodgkin lymphoma may include the following:
Combination chemotherapy with low-dose radiation therapy to involved areas.
A clinical trial of combination chemotherapy with or without low-dose radiation therapy to involved areas.
A clinical trial of new combinations of chemotherapy before low-dose radiation therapy to involved areas.

### High-Risk Childhood Hodgkin Lymphoma

Treatment of high-risk childhood Hodgkin lymphoma may include intensive or high-dose combination chemotherapy with low-dose radiation therapy to involved areas.
Check for clinical trials from NCI's PDQ Cancer Clinical Trials Registry that are now accepting patients with stage III childhood Hodgkin lymphoma and stage IV childhood Hodgkin lymphoma.

### Nodular Lymphocyte Predominant Childhood Hodgkin Lymphoma

Treatment of nodular lymphocyte predominant childhood Hodgkin lymphoma may include the following:
Combination chemotherapy with low-dose radiation therapy to involved areas.
A clinical trial of surgery only, when the lymphoma is stage I and no cancer remains after the surgery.
A clinical trial of combination chemotherapy with or without low-dose radiation therapy to involved areas for patients with stage I or stage II.

### Effects of Treatment:

Late Effects from Childhood and Adolescent Hodgkin Lymphoma Treatment
Children and adolescents may have treatment-related side effects that appear months or years after treatment for Hodgkin lymphoma. Because of these late effects on health and development, regular follow-up exams are important. Late effects may include problems with the following:
Development of sex organs in males.
Fertility (ability to have children).
Thyroid, heart, or lungs.
An increased risk of developing a second primary cancer.
Bone growth and development.

### Ovarian Cancer:

Definition of ovarian cancer: Cancer that forms in tissues of the ovary (one of a pair of female reproductive glands in which the ova, or eggs, are formed). Most ovarian cancers are either ovarian epithelial carcinomas (cancer that begins in the cells on the surface of the ovary) or malignant germ cell tumors (cancer that begins in egg cells).

### New Cases in 2007 in US: 22,430

### Deaths in 2007 in US: 15,280

### Three Types of Ovarian Cancer:

### Ovarian Epithelial

### Ovarian Germ Cell Tumors

Ovarian Low Malignant Potential Tumors-Radiation is not Used as a Treatment for this Cancer

### Survival Rates:

Ovarian cancer accounts for about 3% of all cancers in women.
About 3 in 4 women with ovarian cancer survive at least 1 year after diagnosis. Almost half (45%) of women with ovarian cancer are still alive at least 5 years after diagnosis (this is called the 5-year survival rate). Women younger than 65 have better 5-year survival rates than older women. If ovarian cancer is found (and treated) before the cancer has spread outside the ovary, the 5-year survival rate is 93%. However, less than 20% of all ovarian cancers is found at this early stage.
Ovarian epithelial cancer is a disease in which malignant (cancer) cells form in the tissue covering the ovary.
The following stages are used for ovarian epithelial cancer:

### Stage I

In stage I, cancer is found in one or both of the ovaries and has not spread. Stage I is divided into stage IA, stage IB, and stage IC.
Stage IA: Cancer is found in a single ovary.
Stage IB: Cancer is found in both ovaries.
Stage IC: Cancer is found in one or both ovaries and one of the following is true:
   cancer is found on the outside surface of one or both ovaries; or
   the capsule (outer covering) of the tumor has ruptured (broken open); or
   cancer cells are found in the fluid of the peritoneal cavity (the body cavity that contains most of the organs in the abdomen) or in washings of the peritoneum (tissue lining the peritoneal cavity).

### Stage II

In stage II, cancer is found in one or both ovaries and has spread into other areas of the pelvis. Stage II is divided into stage IIA, stage IIB, and stage IIC.
Stage IIA: Cancer has spread to the uterus and/or the fallopian tubes (the long slender tubes through which eggs pass from the ovaries to the uterus).
Stage IIB: Cancer has spread to other tissue within the pelvis.
Stage IIC: Cancer has spread to the uterus and/or fallopian tubes and/or other tissue within the pelvis and cancer cells are found in the fluid of the peritoneal cavity (the body cavity that contains most of the organs in the abdomen) or in washings of the peritoneum (tissue lining the peritoneal cavity).

### Stage III

In stage III, cancer is found in one or both ovaries and has spread to other parts of the abdomen. Stage III is divided into stage IIIA, stage IIIB, and stage IIIC.
Stage IIIA: The tumor is found only in the pelvis, but cancer cells have spread to the surface of the peritoneum (tissue that lines the abdominal wall and covers most of the organs in the abdomen).
Stage IIIB: Cancer has spread to the peritoneum but is 2 centimeters or smaller in diameter.
Stage IIIC: Cancer has spread to the peritoneum and is larger than 2 centimeters in diameter and/or has spread to lymph nodes in the abdomen.
   Cancer that has spread to the surface of the liver is also considered stage III disease.

### Stage IV

In stage IV, cancer is found in one or both ovaries and has metastasized (spread) beyond the abdomen to other parts of the body. Cancer is found in the tissues of the liver.

### Treatments by Stage:

### Stage I and II Ovarian Epithelial Cancer

Treatment of stage I and stage II ovarian epithelial cancer may include the following:
Total abdominal hysterectomy, bilateral salpingo-oophorectomy, and omentectomy. Lymph nodes and other tissues in the pelvis and abdomen are removed and examined under the microscope to look for cancer cells.
Total abdominal hysterectomy, unilateral salpingo-oophorectomy, and omentectomy. Lymph nodes and other tissues in the pelvis and abdomen are removed and examined under the microscope to look for cancer cells.
A clinical trial of internal or external radiation therapy.
A clinical trial of chemotherapy.
A clinical trial of surgery followed by chemotherapy or watchful waiting (closely monitoring a patient's condition without giving any treatment until symptoms appear or change).
A clinical trial of a new treatment.

### Stage III and IV Ovarian Epithelial Cancer

Treatment of stage III and stage IV ovarian epithelial cancer may be surgery to remove the tumor, total abdominal hysterectomy, bilateral salpingo-oophorectomy, and omentectomy. After surgery, treatment depends on how much tumor remains.
When the tumor that remains is 1 centimeter or smaller, treatment is usually combination chemotherapy, including intraperitoneal (IP) chemotherapy.
When the tumor that remains is larger than 1 centimeter, treatment may include the following:
Combination chemotherapy, including intraperitoneal (IP) chemotherapy.
A clinical trial of combination chemotherapy, including IP chemotherapy, before and after second-look surgery (surgery performed after the initial surgery to determine whether tumor cells remain).
A clinical trial of biologic therapy or targeted therapy following combination chemotherapy.

### Ovarian Germ Cell Tumors

Ovarian germ cell tumor is a disease in which malignant (cancer) cells form in the germ (egg) cells of the ovary.
Germ cell tumors begin in the reproductive cells (egg or sperm) of the body. Ovarian germ cell tumors usually occur in teenage girls or young women and most often affect just one ovary.

### Stages:

### Stage I

In stage I, cancer is found in one or both of the ovaries and has not spread. Stage I is divided into stage IA, stage IB, and stage IC.
Stage IA: Cancer is found in a single ovary.
Stage IB: Cancer is found in both ovaries.
Stage IC: Cancer is found in one or both ovaries and one of the following is true:
   cancer is found on the outside surface of one or both ovaries; or
   the capsule (outer covering) of the tumor has ruptured (broken open); or
   cancer cells are found in the fluid of the peritoneal cavity (the body cavity that contains most of the organs in the abdomen) or in washings of the peritoneum (tissue lining the peritoneal cavity).

### Stage II

In stage II, cancer is found in one or both ovaries and has spread into other areas of the pelvis. Stage II is divided into stage IIA, stage IIB, and stage IIC.
Stage IIA: Cancer has spread to the uterus and/or the fallopian tubes (the long slender tubes through which eggs pass from the ovaries to the uterus).
Stage IIB: Cancer has spread to other tissue within the pelvis.
Stage IIC: Cancer has spread to the uterus and/or fallopian tubes and/or other tissue within the pelvis and cancer cells are found in the fluid of the peritoneal' cavity (the body cavity that contains most of the organs in the abdomen) or in washings of the peritoneum (tissue lining the peritoneal cavity).

### Stage III

In stage III, cancer is found in one or both ovaries and has spread to other parts of the abdomen. Stage III is divided into stage IIIA, stage IIIB, and stage IIIC as follows:
Stage IIIA: The tumor is found only in the pelvis, but cancer cells have spread to the surface of the peritoneum (tissue that lines the abdominal wall and covers most of the organs in the abdomen).
Stage IIIB: Cancer has spread to the peritoneum but is 2 centimeters or smaller in diameter.
Stage IIIC: Cancer has spread to the peritoneum and is larger than 2 centimeters in diameter and/or has spread to lymph nodes in the abdomen.
   Cancer that has spread to the surface of the liver is also considered stage III disease.

### Stage IV

In stage IV, cancer is found in one or both ovaries and has metastasized (spread) beyond the abdomen to other parts of the body. Cancer is found in the tissues of the liver.

### Treatment by Stages:

### Stage I Ovarian Germ Cell Tumors

Treatment depends on whether the tumor is dysgerminoma or another type of germ cell tumor.
Treatment of dysgerminoma may include the following:
Unilateral salpingo-oophorectomy with or without lymphangiography (an x-ray study of the lymph system, the tissues and organs that filter and destroy harmful substances and help fight infection and disease) or CT scan (a series of detailed pictures of areas inside the body, created by a computer linked to an x-ray machine).
Unilateral salpingo-oophorectomy followed by observation (closely monitoring a patient's condition without giving any treatment until symptoms appear or change).
Unilateral salpingo-oophorectomy followed by radiation therapy.
Unilateral salpingo-oophorectomy followed by chemotherapy.
   Treatment of other germ cell tumors may be either:
unilateral salpingo-oophorectomy followed by careful observation; or
unilateral salpingo-oophorectomy, sometimes followed by combination chemotherapy.

### Stage II Ovarian Germ Cell Tumors

Treatment depends on whether the tumor is dysgerminoma or another type of germ cell tumor.
Treatment of dysgerminoma may be either:
total abdominal hysterectomy and bilateral salpingo-oophorectomy followed by radiation therapy or combination chemotherapy; or
unilateral salpingo-oophorectomy followed by chemotherapy.
   Treatment of other germ cell tumors may include the following:
Unilateral salpingo-oophorectomy followed by combination chemotherapy.
Second-look surgery (surgery performed after primary treatment to determine whether tumor cells remain).
A clinical trial of new treatment options.

### Stage III Ovarian Germ Cell Tumors

Treatment depends on whether the tumor is dysgerminoma or another type of germ cell tumor.
Treatment of dysgerminoma may include the following:
Total abdominal hysterectomy and bilateral salpingo-oophorectomy, with removal of as much of the cancer in the pelvis and abdomen as possible.
Unilateral salpingo-oophorectomy followed by chemotherapy.
   Treatment of other germ cell tumors may include the following:
Total abdominal hysterectomy and bilateral salpingo-oophorectomy, with removal of as much of the cancer in the pelvis and abdomen as possible. Chemotherapy will be given before and/or after surgery.
Unilateral salpingo-oophorectomy followed by chemotherapy.
Second-look surgery (surgery performed after primary treatment to determine whether tumor cells remain).
A clinical trial of new treatment options

### Stage IV Ovarian Germ Cell Tumors

Treatment depends on whether the tumor is dysgerminoma or another type of germ cell tumor.
Treatment of dysgerminoma may include the following:
Total abdominal hysterectomy and bilateral salpingo-oophorectomy followed by chemotherapy, with removal of as much of the cancer in the pelvis and abdomen as possible.
Unilateral salpingo-oophorectomy followed by chemotherapy.
   Treatment of other germ cell tumors may include the following:
Total abdominal hysterectomy and bilateral salpingo-oophorectomy, with removal of as much of the cancer in the pelvis and abdomen as possible. Chemotherapy will be given before and/or after surgery.
Unilateral salpingo-oophorectomy followed by chemotherapy.
Second-look surgery (surgery performed after primary treatment to determine whether tumor cells remain).
A clinical trial of new treatment options.

TABLE 7

| Drug (generic name): route | Disease | Dose and Frequency |
|---|---|---|
| Fosamax (alendronate): oral | Osteoporosis | 10 mg daily; 70 mg weekly |
| includes Fosamax + vit D | Osteopenia (preventing osteoporosis) | 5 mg daily; 35 mg weekly |
| | Osteoporosis in men | 10 mg daily; 70 mg weekly |
| includes oral solution | Glucocorticoid Induced Osteoporosis (GIO-steroids) | 5 mg daily; or 10 mg daily for post-menopausal women |
| (powder | Paget's Disease | 40 mg daily for 6 months; retreat as necessary |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 5 mg/day = 70 mg/week to 120 mg/day for ~6 months peri |
| | | radiation-therapy |
| Actonel (risedronate): oral | Osteoporosis | 5 mg daily; 35 mg weekly; 150 mg (75 mg × 2) per month |
| includes Actonel + Ca | Osteopenia (preventing osteoporosis) | 5 mg daily; 35 mg weekly; 150 mg (75 mg × 2) per month |
| | Osteoporosis in men | 35 mg weekly |
| | Glucocorticoid Induced Osteoporosis (GIO-steroids) | 5 mg daily |
| | Paget's Disease | 30 mg daily for two months, retreat as necessary |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 5 mg/day = 35 mg/week = 150 mg/month to 90 mg/day |
| | | for ~6 months peri radiation-therapy |
| Boniva (ibandronate): oral | Osteoporosis | 2.5 mg daily; 150 mg monthly |
| | Osteopenia (preventing osteoporosis) | 2.5 mg daily; 150 mg monthly |
| | Glucocorticoid Induced Osteoporosis (GIO-steroids) | Not approved |
| | Paget's Disease | Not approved |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 2.5 mg/day = 150 mg/month to 50 mg/day for ~6 months |
| | | peri radiation-therapy |
| Boniva (ibandronate): IV | Osteoporosis | 3 mg every 3 months |
| | Osteopenia (preventing osteoporosis) | 3 mg every 3 months |
| | Glucocorticoid Induced Osteoporosis (GIO-steroids) | Not approved |
| | Paget's Disease | Not approved |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 3 mg/month or 2 mg to 6 mg/week for ~6 months peri |
| | | radiation-therapy |
| Zometa (zoledronate): IV | Osteoporosis | Not approved |
| | Osteopenia (preventing osteoporosis) | Not approved |
| | Glucocorticoid Induced Osteoporosis (GIO-steroids) | Not approved |
| | Paget's Disease | Not approved: See Reclast |
| | Hypercalcaemia from Malignancy | 4 mg infusion; re-treat if needed after min 7 days |
| | Multiple Myeloma (osteolytic-bone lesions) | 4 mg infusion every 3-4 weeks |
| | Bone Metastases (osteolytic-bone lesions) | 4 mg infusion every 3-4 weeks |
| | Radiation-induced bone loss range | 4 mg/every 1-2 weeks to 4-5 mg/2 weeks = 10 mg/month |
| | | for ~6 months peri radiation-therapy |
| Reclast (zoledronate): IV | Osteoporosis | 5 mg infusion once a year (alt. 6 months) |
| | Osteopenia (preventing osteoporosis) | |
| | Glucocorticoid Induced Osteoporosis (GIO-steroids) | |
| | Paget's Disease | 5 mg infusion; re-treat as needed |
| | Hypercalcaemia from Malignancy | Not approved: see Zometa |
| | Multiple Myeloma (osteolytic-bone lesions) | Not approved: see Zometa |
| | Bone Metastases (osteolytic-bone lesions) | Not approved: see Zometa |
| | Radiation-induced bone loss range | 4 mg/every 1-2 weeks to 5 mg/2 weeks = 10 mg/month |
| | | for ~6 months peri radiation-therapy |
| Aredia (pamidronate): IV | Osteoporosis | |
| | Osteopenia (preventing osteoporosis) | Not approved |
| | Glucocorticoid Induced Osteoporosis (GIO-steroids) | Not approved |
| | Paget's Disease | 30 mg infusion for 3 days (90 mg total) |
| | Hypercalcaemia from Malignancy | 60-90 mg (single infusion), re-treatment if needed (7 days) |
| | Multiple Myeloma (osteolytic-bone lesions) | 90 mg infusion each month |
| | Bone Metastases (osteolytic-bone lesions) | 90 mg infusion every 3-4 weeks |
| | Radiation-induced bone loss range | 30 mg/every week to 180 mg/month for ~6 months peri |
| | | radiation-therapy |
| Didronel (etidronate): IV | Osteoporosis | Not approved |
| | Osteopenia (preventing osteoporosis) | Not approved |
| | Glucocorticoid Induced Osteoporosis (GIO-steroids) | Not approved |
| | Paget's Disease | Not approved: see oral administration |
| | Hypercalcaemia (generally from Malignancy) | 7.5 mg/kg infusion for three days 22.5 mg/kg total |
| | Multiple Myeloma (osteolytic-bone lesions) | Not approved |
| | Bone Metastases (osteolytic-bone lesions) | Not approved |
| | Radiation-induced bone loss range | 2.5 mg/kg every week to 50 mg/kg week for ~6 months |
| | | peri radiation-therapy |
| Didronel (etidronate): oral | Osteoporosis | Not approved |
| | Osteopenia (preventing osteoporosis) | Not approved |
| | Glucocorticoid Induced Osteoporosis (GIO-steroids) | Not approved |
| | Paget's Disease | 5-10 mg/kg orally daily for 6 months or 11-20 mg/kg daily for |
| | | 3 months |
| | Hypercalcaemia (generally from Malignancy) | Not approved: see IV administration |
| | Multiple Myeloma (osteolytic-bone lesions) | Not approved |
| | Bone Metastases (osteolytic-bone lesions) | Not approved |
| | Radiation-induced bone loss range | 5 mg/kg day to 80 mg/kg day for ~6 months peri |
| | | radiation-therapy |
| Skelid (tiludronate): oral | Osteoporosis | Not approved |
| | Osteopenia (preventing osteoporosis) | Not approved |
| | Glucocorticoid Induced Osteoporosis (GIO-steroids) | Not approved |
| | Paget's Disease | 400 mg daily for three months |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 100 mg/day to 1200 mg/day for ~6 months peri radiation- |
| | | therapy |
| Non-Bisphosphonates: | | |
| Denosumab (anti-RANKL) | Osteoporosis | Yes, FDA trials 6-30 mg/3 mo or 14-210 mg/6 mo |
| Subcutaneous | Osteopenia (preventing osteoporosis) | Yes, FDA trials 6-30 mg/3 mo or 14-210 mg/6 mo |
| Not approved any indication | Glucocorticoid Induced Osteoporosis (GIO-steroids) | No trials as I can tell |
| In FDA trials as indicated | Paget's Disease | No trials as I can tell |
| | Hypercalcaemia from Malignancy | Yes, FDA trials |
| | Multiple Myeloma (osteolytic-bone lesions) | Yes, FDA trials |
| | Bone Metastases (osteolytic-bone lesions) | Yes, FDA trials |
| | Radiation-induced bone loss range | Min = Two 5 mg Injections (3 months apart) or one 14 mg |
| | | injection. |
| | | Max = 90 mg per 4 weeks or 180 mg per 12 weeks for 6 |
| | | months peri-radiotherapy |
| Raloxifene (EVISTA ®): oral | Osteoporosis | 60 mg daily |
| | Osteopenia (preventing osteoporosis) | 60 mg daily |
| | Glucocorticoid Induced Osteoporosis (GIO-steroids) | Not approved |
| | Paget's Disease | Not approved |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 60 mg daily to 240 mg daily for 6 months peri- |
| | | radiotherapy |
| Miacalcin (calcitonin-salmon) | Osteoporosis | 200 IU daily |
| Nasal spray | Osteopenia (preventing osteoporosis) | 200 IU daily |
| | Glucocorticoid Induced Osteoporosis (GIO-steroids) | 200 IU daily |
| | Paget's Disease | Not approved |
| | Hypercalcaemia from Malignancy | Not approved |
| | Multiple Myeloma (osteolytic-bone lesions) | Not approved |
| | Bone Metastases (osteolytic-bone lesions) | Not approved |
| | Radiation-induced bone loss range | 100 IU daily to 400 IU daily for 6 months peri- |
| | | radiotherapy |

Denosumab Literature Summary
Summary of early 2 papers: Doses administered are mg/kg and efficacy demonstrated within a range from 0.1 and 3 mg/kg. 0.01 mg/kg and 0.03 mg/kg were tested and reduced markers for bone formation, but for only temporary periods. 0.3, 1.0 and 3.0 mg/kg performed similarly to standard 90 mg dose of pamidronate, but had a longer efficacy than 30 days. For 70 kg woman 3 mg/kg = 210 mg dose
Summary of 2006 NEJM paper: Doses given in mg-not normalized to patient mass. Compared to standard 70 mg weekly oral alendronate dose. 6, 14 and 30 mg every 3 months and 14, 60 and 210 mg every 6 months.
Summary of 2007 J Clinical Oncology paper: Doses giving in mg-not normalized to body mass. Dmab compared to standard IV dose of zoledronate (4 mg), ibandronate (3 mg) or pamidronate (90 mg) every 4 weeks. Dmab = 30, 120 or 180 mg every 4 weeks or 60 of 180 mg every 12 weeks. All performed similarly at week 2 through 13 weeks post treatment. Dmab data generally pooled.

### REFERENCES

Abe, H., Nakamura, M., Takahashi, S., Maruoka, S., Ogawa, Y. and Sakamoto, K. (1992). Radiation-induced insufficiency fractures of the pelvis: evaluation with 99 mTc-methylene diphosphonate scintigraphy. AJR Am J Roentgenol 158, 599-602.
Aihara, N., Yamaguchi, M. and Kasai, K. (2006). Low-energy irradiation stimulates formation of osteoclast-like cells via RANK expression in vitro. Lasers Med Sci 21, 24-33.
Algur, E., Macklis, R. M. and Hafeli, U. O. (2005). Synergistic cytotoxic effects of zoledronic acid and radiation in human prostate cancer and myeloma cell lines. Int J Radiat Oncol Biol Phys 61, 535-42.
American Cancer Society www.cancer.org
Anderson, N. D., Colyer, R. A. and Riley, L. H., Jr. (1979). Skeletal changes during prolonged external irradiation: alterations in marrow, growth plate and osteoclast populations. Johns Hopkins Med J 145, 73-83.
Anderson, P., Aguilera, D., Pearson, M. and Woo, S. (2008). Outpatient chemotherapy plus radiotherapy in sarcomas: improving cancer control with radiosensitizing agents. Cancer Control 15, 38-46.
Arrington, S. A., Damron, T. A., Mann, K. A. and Allen, M. J. (2007). Concurrent administration of zoledronic acid and irradiation leads to improved bone density, biomechanical strength, and microarchitecture in a mouse model of tumor-induced osteolysis. J Surg Oncol.
Bagshawe, K. D. (1989). The First Bagshawe lecture. Towards generating cytotoxic agents at cancer sites. Br J Cancer 60, 275-81.
Bagshawe, K. D., Springer, C. J., Searle, F., Antoniw, P., Sharma, S. K., Melton, R. G. and Sherwood, R. F. (1988). A cytotoxic agent can be generated selectively at cancer sites. Br J Cancer 58, 700-3.
Bandstra, E. R., Pecaut, M. J., Anderson E. R., Willey, J. S., DeCarlo, F., Stock, S. R., Gridley, D. S., Nelson, G. A., Levine, H. G., Bateman, T. A. (2008). Long-term dose response of trabecular bone in mice to proton radiation. Radiation Research, PMID 18494551, 169:607-14.
Battelli, M. G., Abbondanza, A., Tazzari, P. L., Bolognesi, A., Lemoli, R. M. and Stirpe, F. (1992). T lymphocyte killing by a xanthine-oxidase-containing immunotoxin. Cancer Immunol Immunother 35, 421-5.
Baxter, N. N., Habermann, E. B., Tepper, J. E., Durham, S. B. and Virnig, B. A. (2005). Risk of pelvic fractures in older women following pelvic irradiation. JAMA 294, 2587-93.
Bliss, P., Parsons, C. A. and Blake, P. R. (1996). Incidence and possible aetiological factors in the development of pelvic insufficiency fractures following radical radiotherapy. Br J Radiol 69, 548-54.
Blomlie, V., Rofstad, E. K., Talle, K., Sundfor, K., Winderen, M. and Lien, H. H. (1996). Incidence of radiation-induced insufficiency fractures of the female pelvis: evaluation with MR imaging. AJR Am J Roentgenol 167, 1205-10.
Body, J. J., Facon, T., Coleman, R. E., Lipton, A., Geurs, F., Fan, M., Holloway, D., Peterson, M. C. and Bekker, P. J. (2006). A study of the biological receptor activator of nuclear factor-kappaB ligand inhibitor, denosumab, in patients with multiple myeloma or bone metastases from breast cancer. Clin Cancer Res 12, 1221-8.
Bolek, T. W., Marcus, R. B., Jr., Mendenhall, N. P., Scarborough, M. T. and Graham-Pole, J. (1996). Local control and functional results after twice-daily radiotherapy for Ewing's sarcoma of the extremities. Int J Radiat Oncol Biol Phys 35, 687-92.
Borah, B., Dufresne, T. E., Chmielewski, P. A., Gross, G. J., Prenger, M. C. and Phipps, R. J. (2002). Risedronate preserves trabecular architecture and increases bone strength in vertebra of ovariectomized minipigs as measured by three-dimensional microcomputed tomography. J Bone Miner Res 17, 1139-47.
Brown, V. I. and Greene, M. I. (1991). Molecular and cellular mechanisms of receptor-mediated endocytosis. DNA Cell Biol 10, 399-409.
Brownstein, C. M., Mertens, A. C., Mitby, P. A., Stovall, M., Qin, J., Heller, G., Robison, L. L. and Sklar, C. A. (2004). Factors that affect final height and change in height standard deviation scores in survivors of childhood cancer treated with growth hormone: a report from the childhood cancer survivor study. J Clin Endocrinol Metab 89, 4422-7.
Chapurlat, R. D. and Delmas, P. D. (2006). Drug insight: Bisphosphonates for postmenopausal osteoporosis. Nat Clin Pract Endocrinol Metab 2, 211-9.
Chirgwin, J. M., Mohammad, K. S, and Guise, T. A. (2004). Tumor-bone cellular interactions in skeletal metastases. J Musculoskelet Neuronal Interact 4, 308-18.
Clines, G. A. and Guise, T. A. (2004). Mechanisms and treatment for bone metastases. Clin Adv Hematol Oncol 2, 295-302.
Clines, G. A. and Guise, T. A. (2005). Hypercalcaemia of malignancy and basic research on mechanisms responsible for osteolytic and osteoblastic metastasis to bone. Endocr Relat Cancer 12, 549-83.
Darzy, K. H. and Shalet, S. M. (2005). Hypopituitarism as a consequence of brain tumours and radiotherapy. Pituitary 8, 203-11.
Davies, J. H., Evans, B. A., Jenney, M. E. and Gregory, J. W. (2005). Skeletal morbidity in childhood acute lymphoblastic leukaemia. Clin Endocrinol (Oxf) 63, 1-9.
Diamond, T. H., Higano, C. S., Smith, M. R., Guise, T. A. and Singer, F. R. (2004). Osteoporosis in men with prostate carcinoma receiving androgen-deprivation therapy: recommendations for diagnosis and therapies. Cancer 100, 892-9.
Duncan, G. G., Corbett, T., Lukka, H., Warde, P. and Pickles, T. (2006). GU radiation oncologists consensus on bone loss from androgen deprivation. Can J Urol 13, 2962-6.
Erben, R. G., Mosekilde, L., Thomsen, J. S., Weber, K., Stahr, K., Leyshon, A., Smith, S. Y. and Phipps, R. (2002). Prevention of bone loss in ovariectomized rats by combined treatment with risedronate and 1alpha,25-dihydroxyvitamin D3. J Bone Miner Res 17, 1498-511.
Ergun, H. and Howland, W. J. (1980). Postradiation atrophy of mature bone. CRC Crit. Rev Diagn Imaging 12, 225-43.
Facchini, G., Caraglia, M., Santini, D., Nasti, G., Ottaiano, A., Striano, S., Maiolino, P., Ruberto, M., Fiore, F., Tonini, G. et al. (2007). The clinical response on bone metastasis from breast and lung cancer during treatment with zoledronic acid is inversely correlated to skeletal related events (SRE). J Exp Clin Cancer Res 26, 307-12.
Fleisch, H. (2000). Bisphosphonates in Bone Disease: From the Laboratory to the Patient, 4^{th} Edition, San Diego: Academic Press.
Fukuda, S., Iida, H. and Yan, X. (2002). Preventive effects of running exercise on bones in heavy ion particle irradiated rats. J Radiat Res (Tokyo) 43 Suppl, S233-8.
Furstman, L. L. (1972). Effect of radiation on bone. J Dent Res 51, 596-604.
Gal, T. J., Munoz-Antonia, T., Muro-Cacho, C. A. and Klotch, D. W. (2000). Radiation effects on osteoblasts in vitro: a potential role in osteoradionecrosis. Arch Otolaryngol Head Neck Surg 126, 1124-8.
Grigsby, P. W., Roberts, H. L. and Perez, C. A. (1995). Femoral neck fracture following groin irradiation. Int J Radial Oncol Biol Phys 32, 63-7.
Guise, T. A. (2006). Bone loss and fracture risk associated with cancer therapy. Oncologist 11, 1121-31.
Guise, T. A., Kozlow, W. M., Heras-Herzig, A., Padalecki, S. S., Yin, J. J. and Chirgwin, J. M. (2005). Molecular mechanisms of breast cancer metastases to bone. Clin Breast Cancer 5 Suppl, S46-53.
Guise, T. A., Mohammad, K. S., Clines, G., Stebbins, E. G., Wong, D. H., Higgins, L. S., Vessella, R., Corey, E., Padalecki, S., Suva, L. et al. (2006). Basic mechanisms responsible for osteolytic and osteoblastic bone metastases. Clin Cancer Res 12, 6213s-6216s.
Guise, T. A. and Mundy, G. R. (1998). Cancer and bone. Endocr Rev 19, 18-54.
Guise, T. A., Oefelein, M. G., Eastham, J. A., Cookson, M. S., Higano, C. S, and Smith, M. R. (2007). Estrogenic side effects of androgen deprivation therapy. Rev Urol 9, 163-80.
Gungor, T., Hedlund, T., Hulth, A. and Johnell, O. (1982). The effect of irradiation on osteoclasts with or without transplantation of hematopoietic cells. Acta Orthop Scand 53, 333-7.
Hamilton, S H, Pecaut, M J, Gridley D S, Travis, N S, Bandstra, E R, Willey, J S, Nelson, G A, Bateman T A A murine model for bone loss from therapeutic and space-relevant sources of radiation. J Applied Physiology, PMID 16741258, 101: 789-793; 2006.
Hansen, T., Kirkpatrick, C. J., Walter, C. and Kunkel, M. (2006). Increased numbers of osteoclasts expressing cysteine proteinase cathepsin K in patients with infected osteoradionecrosis and bisphosphonate-associated osteonecrosis—a paradoxical observation? Virchows Arch 449, 448-54.
Hartsell, W. F., Scott, C. B., Bruner, D. W., Scarantino, C. W., Ivker, R. A., Roach, M., 3rd, Suh, J. H., Demas, W. F., Movsas, B., Petersen, I. A. et al. (2005). Randomized trial of short-versus long-course radiotherapy for palliation of painful bone metastases. J Natl Cancer Inst 97, 798-804.
Higano, C. S. (2008). Androgen-deprivation-therapy-induced fractures in men with nonmetastatic prostate cancer: what do we really know? Nat Clin Pract Urol 5, 24-34.
Hirbe, A., Morgan, E. A., Uluckan, O. and Weilbaecher, K. (2006). Skeletal complications of breast cancer therapies. Clin Cancer Res 12, 6309s-6314s.
Hofbauer, L. C. and Heufelder, A. E. (2001). Role of receptor activator of nuclear factor-kappaB ligand and osteoprotegerin in bone cell biology. J Mol Med 79, 243-53.
Hopewell, J. W. (2003). Radiation-therapy effects on bone density. Med Pediatr Oncol 41, 208-11.
Horton, J. A., Margulies, B. S., Strauss, J. A., Bariteau, J. T., Damron, T. A., Spadaro, J. A. and Farnum, C. E. (2006). Restoration of growth plate function following radiotherapy is driven by increased proliferative and synthetic activity of expansions of chondrocytic clones. J Orthop Res 24, 1945-56.
Hosokawa, Y., Sakakura, Y., Tanaka, L., Okumura, K., Yajima, T. and Kaneko, M. (2007). Effects of local and whole body irradiation on appearance of osteoclasts during wound healing of tooth extraction sockets in rats. J Radiat Res (Tokyo) 48, 273-80.
Howland, W. J., Loeffler, R. K., Starchman, D. E. and Johnson, R. G. (1975). Postirradiation atrophic changes of bone and related complications. Radiology 117, 677-85.
Hughes, B. J., Kennel, S., Lee, R. and Huang, L. (1989). Monoclonal antibody targeting of liposomes to mouse lung in vivo. Cancer Res 49, 6214-20.
Huh, S. J., Kim, B., Kang, M. K., Lee, J. E., Lim do, H., Park, W., Shin, S. S, and Ahn, Y. C. (2002). Pelvic insufficiency fracture after pelvic irradiation in uterine cervix cancer. Gynecol Oncol 86, 264-8.
Iwamoto, J., Seki, A., Takeda, T., Sato, Y., Yamada, H., Shen, C. L. and Yeh, J. K. (2006). Preventive effects of risedronate and calcitriol on cancellous osteopenia in rats treated with high-dose glucocorticoid. Exp Anim 55, 349-55.
Jacobsson, M., Kalebo, P., Tjellstrom, A. and Turesson, I. (1987). Bone cell viability after irradiation. An enzyme histochemical study. Acta Oncol 26, 463-5.
Keyak, J. H., Koyama, A. K., Leblanc, A., Lu, Y. and Lang, T. F. (2008). Reduction in proximal femoral strength due to long-duration spaceflight. Bone Epub ahead of print, PMID: 19100348.
Konski, A. and Sowers, M. (1996). Pelvic fractures following irradiation for endometrial carcinoma. Int J Radiat Oncol Biol Phys 35, 361-7.
Kostenuik, P. J. and Shalhoub, V. (2001). Osteoprotegerin: a physiological and pharmacological inhibitor of bone resorption. Curr Pharm Des 7, 613-35.
Klinck, R. J., Campbell, G. M. and Boyd, S. K. (2008). Radiation effects on bone architecture in mice and rats resulting from in vivo micro-computed tomography scanning. Med Eng Phys 30, 888-95.
Krasin, M. J., Xiong, X., Wu, S, and Merchant, T. E. (2005). The effects of external beam irradiation on the growth of flat bones in children: modeling a dose-volume effect. Int J Radiat Oncol Biol Phys 62, 1458-63.
Lacey, D. L., Timms, E., Tan, H. L., Kelley, M. J., Dunstan, C. R., Burgess, T., Elliott, R., Colombero, A., Elliott, G., Scully, S. et al. (1998). Osteoprotegerin ligand is a cytokine that regulates osteoclast differentiation and activation. Cell 93, 165-76.
Lai, T. J., Hsu, S. F., Li, T. M., Hsu, H. C., Lin, J. G., Hsu, C. J., Chou, M. C., Lee, M. C., Yang, S. F. and Fong, Y. C. (2007). Alendronate inhibits cell invasion and MMP-2 secretion in human chondrosarcoma cell line. Acta Pharmacol Sin 28, 1231-5.
Lane, N. E. (2006). Epidemiology, etiology, and diagnosis of osteoporosis. Am J Obstet Gynecol 194, S3-11.
Lang, T., LeBlanc, A., Evans, H., Lu, Y., Genant, H. and Yu, A. (2004). Cortical and trabecular bone mineral loss from the spine and hip in long-duration spaceflight. J Bone Miner Res 19, 1006-12.
Lang, T. F., Leblanc, A. D., Evans, H. J. and Lu, Y. (2006). Adaptation of the proximal femur to skeletal reloading after long-duration spaceflight. J Bone Miner Res 21, 1224-30.
Lawrence, T. S., Davis, M. A., Hough, A. and Rehemtulla, A. (2001). The role of apoptosis in 2′,2′-difluoro-2′-deoxycytidine (gemcitabine)-mediated radiosensitization. Clin Cancer Res 7, 314-9.
Lewiecki, E. M., Miller, P. D., McClung, M. R., Cohen, S. B., Bolognese, M. A., Liu, Y., Wang, A., Siddhanti, S, and Fitzpatrick, L. A. (2007). Two-year treatment with denosumab (AMG 162) in a randomized phase 2 study of postmenopausal women with low BMD. J Bone Miner Res 22, 1832-41.
Li, Y. Y., Chang, J. W., Chou, W. C., Liaw, C. C., Wang, H. M., Huang, J. S., Wang, C. H. and Yeh, K. Y. (2008). Zoledronic acid is unable to induce apoptosis, but slows tumor growth and prolongs survival for non-small-cell lung cancers. Lung Cancer 59, 180-91.
Lipton, A., Steger, G. G., Figueroa, J., Alvarado, C., Solal-Celigny, P., Body, J. J., de Boer, R, Berardi, R., Gascon, P., Tonkin, K. S. et al. (2007). Randomized active-controlled phase II study of denosumab efficacy and safety in patients with breast cancer-related bone metastases. J Clin Oncol 25, 4431-7.
Litzinger, D. C. and Huang, L. (1992). Biodistribution and immunotargetability of ganglioside-stabilized dioleoylphosphatidylethanolamine liposomes. Biochim Biophys Acta 1104, 179-87.
Lobato, J. V., Rodrigues, J. M., Cavaleiro, M. V., Lobato, J. M., Xavier, L., Santos, J. D. and Mauricio, A. C. (2007). Maxilla osseus sequestre and oral exposure: effects of the treatment of multiple myeloma with bisphosphonates. Acta Med Port 20, 185-92.
Ma, Y. F., Lin, B. Y., Jee, W. S., Lin, C. H., Chen, Y. Y., Ke, H. Z. and Li, X. J. (1997). Prostaglandin E2 (PGE2) and risedronate was superior to PGE2 alone in maintaining newly added bone in the cortical bone site after withdrawal in older intact rats. J Bone Miner Res 12, 267-75.
Margulies, B., Morgan, H., Allen, M., Strauss, J., Spadaro, J. and Damron, T. (2003). Transiently increased bone density after irradiation and the radioprotectant drug amifostine in a rat model. Am J Clin Oncol 26, e106-14.
Mason, M. D., Sydes, M. R., Glaholm, J., Langley, R. E., Huddart, R. A., Sokal, M., Stott, M., Robinson, A. C., James, N. D., Parmar, M. K. et al. (2007). Oral sodium clodronate for nonmetastatic prostate cancer—results of a randomized double-blind placebo-controlled trial: Medical Research Council PRO4 (ISRCTN61384873). J Natl Cancer Inst 99, 765-76.
McClung, M. R., Lewiecki, E. M., Cohen, S. B., Bolognese, M. A., Woodson, G. C., Moffett, A. H., Peacock, M., Miller, P. D., Lederman, S, N., Chesnut, C. H. et al. (2006). Denosumab in postmenopausal women with low bone mineral density. N Engl J Med 354, 821-31.
Min, H., Morony, S., Sarosi, I., Dunstan, C. R., Capparelli, C., Scully, S., Van, G., Kaufman, S., Kostenuik, P. J., Lacey, D. L. et al. (2000). Osteoprotegerin reverses osteoporosis by inhibiting endosteal osteoclasts and prevents vascular calcification by blocking a process resembling osteoclastogenesis. J Exp Med 192, 463-74.
Mitchell, M. J. and Logan, P. M. (1998). Radiation-induced changes in bone. Radiographics 18, 1125-36; quiz 1242-3.
Moreno, A., Clemente, J., Crespo, C., Martinez, A., Navarro, M., Fernandez, L., Minguell, J., Vazquez, G. and Andreu, F. J. (1999). Pelvic insufficiency fractures in patients with pelvic irradiation. Int J Radiat Oncol Biol Phys 44, 61-6.
Morgan, M. A., Parsels, L. A., Maybaum, J. and Lawrence, T. S. (2008). Improving gemcitabine-mediated radiosensitization using molecularly targeted therapy: a review. Clin Cancer Res 14, 6744-50.
Mundy, G. R. and Guise, T. A. (1997). Hypercalcemia of malignancy. Am J Med 103, 134-45.
National Cancer Institute www.cancer.gov
National Osteoporosis Foundation www.nof.org
Nishiyama, K., Inaba, F., Higashirara, T., Kitatani, K. and Kozuka, T. (1992). Radiation osteoporosis—an assessment using single energy quantitative computed tomography. Eur Radiol 2, 322-325.
Nyaruba, M. M., Yamamoto, I., Kimura, H. and Morita, R. (1998). Bone fragility induced by X-ray irradiation in relation to cortical bone-mineral content. Acta Radiol 39, 43-6.
Ogino, I., Okamoto, N., Ono, Y., Kitamura, T. and Nakayama, H. (2003). Pelvic insufficiency fractures in postmenopausal woman with advanced cervical cancer treated by radiotherapy. Radiother Oncol 68, 61-7.
Oh, D., Huh, S. J., Nam, H., Park, W., Han, Y., Lim do, H., Ahn, Y. C., Lee, J. W., Kim, B. G., Bae, D. S. et al. (2008). Pelvic insufficiency fracture after pelvic radiotherapy for cervical cancer: analysis of risk factors. Int J Radiat Oncol Biol Phys 70, 1183-8.
Otomo, H., Sakai, A., Ikeda, S., Tanaka, S., Ito, M., Phipps, R. J. and Nakamura, T. (2004). Regulation of mineral-to-matrix ratio of lumbar trabecular bone in ovariectomized rats treated with risedronate in combination with or without vitamin K2. J Bone Miner Metab 22, 404-14.
Overgaard, M. (1988). Spontaneous radiation-induced rib fractures in breast cancer patients treated with postmastectomy irradiation. A clinical radiobiological analysis of the influence of fraction size and dose-response relationships on late bone damage. Acta Oncol 27, 117-22.
Pierce, S. M., Recht, A., Lingos, T. I., Abner, A., Vicini, F., Silver, B., Herzog, A. and Harris, J. R. (1992). Long-term radiation complications following conservative surgery (CS) and radiation therapy (RT) in patients with early stage breast cancer. Int J Radiat Oncol Biol Phys 23, 915-23.
Pietersz, G. A. and McKenzie, I. F. (1992). Antibody conjugates for the treatment of cancer. Immunol Rev 129, 57-80.
Pitkanen, M. A. and Hopewell, J. W. (1983). Functional changes in the vascularity of the irradiated rat femur. Implications for late effects. Acta Radiol Oncol 22, 253-6.
Roffler, S. R., Wang, S. M., Chem, J. W., Yeh, M. Y. and Tung, E. (1991). Anti-neoplastic glucuronide prodrug treatment of human tumor cells targeted with a monoclonal antibody-enzyme conjugate. Biochem Pharmacol 42, 2062-5.
Roodman, G. D. (2001). Biology of osteoclast activation in cancer. J Clin Oncol 19, 3562-71.
Saad, F., Higano, C. S., Sartor, O., Colombel, M., Murray, R., Mason, M. D., Tubaro, A. and Schulman, C. (2006). The role of bisphosphonates in the treatment of prostate cancer: recommendations from an expert panel. Clin Genitourin Cancer 4, 257-62.
Sams, A. (1966a). The effect of 2000 r of x-rays on the acid and alkaline phosphatase of mouse tibiae. Int J Radiat Biol Relat Stud Phys Chem Med 10, 123-40.
Sawajiri, M. and Mizoe, J. (2003). Changes in bone volume after irradiation with carbon ions. Radiat Environ Biophys 42, 101-6.
Sawajiri, M., Mizoe, J. and Tanimoto, K. (2003). Changes in osteoclasts after irradiation with carbon ion particles. Radiat Environ Biophys 42, 219-23.
Sawajiri, M., Nomura, Y., Bhawal, U. K., Nishikiori, R., Okazaki, M., Mizoe, J. and Tanimoto, K. (2006). Different effects of carbon ion and gamma-irradiation on expression of receptor activator of NF-kB ligand in MC₃T3-E1 osteoblast cells. Bull Exp Biol Med 142, 618-24.
Scheven, B. A., Burger, E. H., Kawilarang-de Haas, E. W., Wassenaar, A. M. and Nijweide, P. J. (1985). Effects of ionizing irradiation on formation and resorbing activity of osteoclasts in vitro. Lab Invest 53, 72-9.
Scheven, B. A., Wassenaar, A. M., Kawilarang-de Haas, E. W. and Nijweide, P. J. (1987a). Comparison of direct and indirect radiation effects on osteoclast formation from progenitor cells derived from different hemopoietic sources. Radiat Res 111, 107-18.
Scheven, B. A., Wassenaar, A. M., Kawilarang-de Haas, E. W. and Nijweide, P. J. (1987b). Direct and indirect radiation effects on osteoclast formation in vitro. Bone Miner 2, 291-300.
Senter, P. D., Wallace, P. M., Svensson, H. P., Vrudhula, V. M., Kerr, D. E., Hellstrom, I. and Hellstrom, K. E. (1993). Generation of cytotoxic agents by targeted enzymes. Bioconjug Chem 4, 3-9.
Senter, P. D., Su, P. C., Katsuragi, T., Sakai, T., Cosand, W. L., Hellstrom, I. and Hellstrom, K. E. (1991). Generation of 5-fluorouracil from 5-fluorocytosine by monoclonal antibody-cytosine deaminase conjugates. Bioconjug Chem 2, 447-51.
Simonet, W. S., Lacey, D. L., Dunstan, C. R., Kelley, M., Chang, M. S., Luthy, R., Nguyen, H. Q., Wooden, S., Bennett, L., Boone, T. et al. (1997). Osteoprotegerin: a novel secreted protein involved in the regulation of bone density. Cell 89, 309-19.
Small, W., Jr. and Kachnic, L. (2005). Postradiotherapy pelvic fractures: cause for concern or opportunity for future research? JAMA 294, 2635-7.
Smith, M. R. (2008). Osteoclast targeted therapy for prostate cancer: bisphosphonates and beyond. Urol Oncol 26, 420-5.
Staatz, G., Honnef, D., Kochs, A., Hohi, C., Schmidt, T., Rohrig, H. and Gunther, R. W. (2006). Evaluation of femoral head vascularization in slipped capital femoral epiphysis before and after cannulated screw fixation with use of contrast-enhanced MRI: initial results. Eur Radiol.
Sugimoto, M., Takahashi, S., Toguchida, J., Kotoura, Y., Shibamoto, Y. and Yamamuro, T. (1991). Changes in bone after high-dose irradiation. Biomechanics and histomorphology. J Bone Joint Surg Br 73, 492-7.
Szymczyk, K. H., Shapiro, I. M. and Adams, C. S. (2004). Ionizing radiation sensitizes bone cells to apoptosis. Bone 34, 148-56.
Takahashi, S., Sugimoto, M., Kotoura, Y., Sasai, K., Oka, M. and Yamamuro, T. (1994). Long-term changes in the haversian systems following high-dose irradiation. An ultrastructural and quantitative histomorphological study. J Bone Joint Surg Am 76, 722-38.
Tivesten, A., Moverare-Skrtic, S., Chagin, A., Venken, K., Salmon, P., Vanderschueren, D., Savendahl, L., Holmang, A. and Ohlsson, C. (2004). Additive protective effects of estrogen and androgen treatment on trabecular bone in ovariectomized rats. J Bone Miner Res 19, 1833-9.
Tsay, T. P., Chen, M. H. and Oyen, O. J. (1999). Osteoclast activation and recruitment after application of orthodontic force. Am J Orthod Dentofacial Orthop 115, 323-30.
Tsay, T. P., Chen, M. H., Oyen, O. J., Hahn, S. S, and Marty, J. J. (1995). The effect of cobalt-60 irradiation on bone marrow cellularity and alveolar osteoclasts. Proc Natl Sci Counc Repub China B 19, 185-95.
Van Poznak, C. H. (2002). The use of bisphosphonates in patients with breast cancer. Cancer Control 9, 480-9.
Weilbaecher, K. N. (2000). Mechanisms of osteoporosis after hematopoietic cell transplantation. Biol Blood Marrow Transplant 6, 165-74.
Willey, J. S., Lloyd, S. A., Robbins, M. E., Bourland, J. D., Smith-Sielicki, H., Bowman, L. C., Norrdin, R. W. and Bateman, T. A. (2008). Early increase in osteoclast number in mice after whole-body irradiation with 2 Gy X rays. Radiat Res 170, 388-92.
Williams, H. J. and Davies, A. M. (2006). The effect of X-rays on bone: a pictorial review. Eur Radiol 16, 619-33.

What is claimed is:

1. A method of preventing ionizing radiation-associated loss of bone mass, density or strength in a subject, comprising administering to the subject an amount of an antiresorptive, osteoclast inhibiting, compound sufficient to prevent loss of bone density, mass or strength.

2. A method of preventing loss of bone mass, density or strength in patients receiving or about to receive radiation therapy, comprising administering to the subject an amount of an antiresorptive compound sufficient to prevent loss of bone mass and/or bone density.

3. A method of preventing ionizing radiation-associated increase in the number or activity of osteoclasts in a subject, comprising administering to the subject an amount of an antiresorptive compound sufficient to reduce osteoclast numbers.

4. The method of claim 1, wherein the patients are adults of age 18 or older.

5. The method of claim 1, wherein the total radiation dose to any part of the body is greater than 1 Gy.

6. The method of claim 1, wherein the subject is receiving or is about to receive radiotherapy for a cancer or tumor selected from the group consisting of prostate, cervical, uterine, bladder, urinary, ovarian, anal, rectal, colon, lung, stomach, esophagus, breast, leukemia, and lymphoma.

7. The method of claim 1, wherein the bone exposed to ionizing radiation is a skeletal component selected from the group consisting of proximal femur, hips, pelvis, vertebral components of the spine, and ribs.

8. The method of claim 1, wherein the total amount of antiresorptive agent administered is at least 25% more than the amount of the same antiresporptive agent administered for the treatment of non-radiation-induced osteoporosis.

9. The method of claim 1, wherein the antiresorptive agent is administered after diagnosis of cancer.

10. The method of claim 1, wherein the period of treatment is peri-radiation, beginning one day to 4 months prior to beginning of the radiation therapy period and continues up to 3 months after termination of radiation therapy.

11. The method of claim 1, wherein the antiresorptive compound is selected from the group consisting of alendronate, risedronate, ibandronate, zoledronate, pamidronate, etidronate, tiludronate, EVISTA®, denosumab, calcitonin, and anti-RANKL.

12. The method of claim 1, further comprising administering a calcium supplement.

13. The method of claim 1, further comprising administration of calcitriol or a vitamin D supplement.

14. The method of claim 1, wherein the antiresorptive compound is administered orally.

15. The method of claim 1, wherein the antiresorptive compound is administered intravenously.

16. The method of claim 1, wherein the antiresorptive compound is administered subcutaneously.

17. The method of claim 1, wherein the antiresorptive compound is administered during the peri-radiation therapy period.

18. The method of claim 10, wherein the peri-radiation therapy period is a contiguous 6-month period including a period of radiation therapy.

19. The method of claim 10, wherein the peri-radiation-therapy period begins about one day to 4 months prior to the initiation of radiation therapy.

20. The method of claim 10, wherein the peri-radiation period begins after the diagnosis of cancer.

21. The method of claim 10, wherein the peri-radiation-therapy period includes a period of up to two months after radiotherapy has concluded.

22. The method of claim 1, wherein the amount of antiresorptive compound effective to prevent radiation induced bone loss in a patient that has not previously received an antiresorptive agent is at least 20 micrograms/kg/day orally.

23. The method of claim 1, wherein the amount of antiresorptive compound effective to prevent radiation induced bone loss in a patient that has not previously received an antiresorptive agent is at least a single administration of 8 micrograms/kg intravenously.

24. The method of claim 1, wherein the amount of antiresorptive compound effective to prevent radiation induced bone loss in a patient that has not previously received an antiresorptive agent is at least a single administration of 10 micrograms/kg subcutaneously, intramuscularly, or by other injection method.

25. The method of claim 1, wherein the antiresorptive compound is a bisphosphonate, or a pharmaceutically acceptable salt thereof, having the formula: 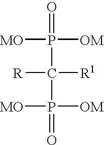
wherein M represents hydrogen or a pharmaceutically acceptable cation capable of providing electronic neutrality to the molecule;
R is a unit having the formula:
(L¹)_{x-Z}
the index x is 0 or 1;
Z is a unit chosen from:
i) C₁-C₁₂ substituted or unsubstituted linear, branched, or cyclic alkyl, alkenyl, and alkynyl;
ii) C₆ or C₁₀ substituted or unsubstituted aryl;
iii) C₁-C₉ substituted or unsubstituted heterocyclic as further defined herein;
iv) C₁-C₁₁ substituted or unsubstituted heteroaryl as further defined herein;
v) —[C(R^{2a})(R^{2b})]_{y}OR³;
a) wherein R³ is chosen from:
b) —H;
c) C₁-C₁₂ substituted or unsubstituted linear, branched, or cyclic alkyl;
d) C₆ or C₁₀ substituted or unsubstituted aryl or alkylenearyl;
e) C₁-C₉ substituted or unsubstituted heterocyclic;
f) C₁-C₁₁ substituted or unsubstituted heteroaryl;
vi) —[C(R^{2a})(R^{2b})]_{y}N(R^{4a})(R^{4b});
a) wherein R^{4a} and R^{4b} are each independently chosen from:
i) —H;
ii) —OR⁵;
R⁵ is hydrogen or C₁-C₄ linear alkyl;
b) C₁-C₁₂ substituted or unsubstituted linear, branched, or cyclic alkyl;
c) C₆ or C₁₀ substituted or unsubstituted aryl;
d) C₁-C₉ substituted or unsubstituted heterocyclic;
e) C₁-C₁₁ substituted or unsubstituted heteroaryl; or
f) R^{4a} and R^{4b} can be taken together to form a substituted or unsubstituted ring having from 3 to 10 carbon atoms and from 0 to 3 heteroatoms chosen from oxygen, nitrogen, and sulfur;
vii) —[C(R^{2a})(R^{2b})]_{y}C(O)R⁶;
a) wherein R⁶ is chosen from:
i) C₁-C₁₂ substituted or unsubstituted linear, branched, or cyclic alkyl;
ii) —OR⁷;
R⁷ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
b) —N(R^{8a})(R^{8b}); and
R^{8a} and R^{8b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{8a} and R^{8b} can be taken together to form a substituted or unsubstituted ring having from 3 to 10 carbon atoms and from 0 to 3 heteroatoms chosen from oxygen, nitrogen, and sulfur;
viii) —[C(R^{2a})(R^{2b})]_{y}OC(O)R⁹;
wherein R⁹ is chosen from:
a) C₁-C₁₂ substituted or unsubstituted linear, branched, or cyclic alkyl;
b) —N(R^{10a})(R^{10b}); and
R^{10a} and R^{10b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{10b} can be taken together to form a substituted or unsubstituted ring having from 3 to 10 carbon atoms and from 0 to 3 heteroatoms chosen from oxygen, nitrogen, and sulfur;
ix) —[C(R^{2a})(R^{2b})]_{y}NR¹¹C(O)R¹²;
wherein R¹¹ is chosen from:
a) —H; and
b) C₁-C₄ substituted or unsubstituted linear, branched, or cyclic alkyl;
c) wherein R¹² is chosen from:
i) C₁-C₁₂ substituted or unsubstituted linear, branched, or cyclic alkyl; and
ii) —N(R^{13a})(R^{13b});
R^{13a} and R^{13b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{13a} and R^{13b} can be taken together to form a substituted or unsubstituted ring having from 3 to 10 carbon atoms and from 0 to 3 heteroatoms chosen from oxygen, nitrogen, and sulfur;
x) —[C(R^{2a})(R^{2b})]_{y}CN;
xi) —[C(R^{2a})(R^{2b})]_{y}NO₂;
xii) —[C(R^{2a})(R^{2b})]_{y}SO₂R¹⁴;
wherein R¹⁴ is hydrogen, hydroxyl, substituted or unsubstituted C₁-C₄ linear or branched alkyl; substituted or unsubstituted C₆, C₁₀, or C₁₋₄ aryl; C₇-C₁₅ alkylenearyl; C₁-C₉ substituted or unsubstituted heterocyclic; or C₁-C₁₁ substituted or unsubstituted heteroaryl;
xiii) halogen; and
xiv) —SR'⁵;
R¹⁵ is chosen from:
i) C₁-C₁₂ substituted or unsubstituted linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) C₆ or C₁₀ substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
R^{2a} and R^{2b} are each independently hydrogen or C₁-C₄ alkyl; and
the index y is from 0 to 5;
L¹ is chosen from:
i) —[C(R^{16a}R^{16b})]ₘ—;
ii) —OH; or
iii) halogen;
R^{15a} and R^{16b} are each independently chosen from hydrogen or methyl; and the index
m is from 1 to 20; and
R¹ is a unit chosen from:
i) hydrogen;
ii) —OH;
iii) halogen; and
iv) methyl.

26. The method of claim 25, wherein R¹ is —OH.

27. The method of claim 25, wherein R¹ is hydrogen.

28. The method of claim 25, wherein L¹ is chosen from:
i) —CH₂—;
ii) —CH₂CH₂—;
iii) —CH₂CH₂CH₂—;
iv) —CH₂CH₂CH₂CH₂—; and
V) —CH₂CH₂CH₂CH₂CH₂—.

29. The method of claim 25, wherein the bisphosphonate is chosen from:

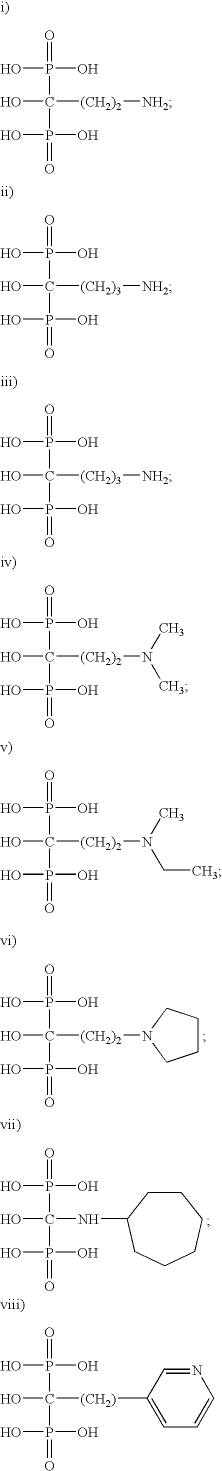

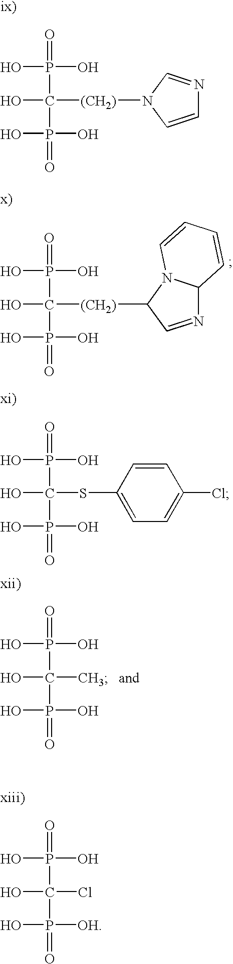

30. A method of treating cancer in a subject comprising:
a) administering to the subject an amount of an antiresorptive agent effective to prevent or reduce radiation-induce loss of bone mass, bone density or bone strength; and
b) administering to the subject radiation therapy to treat the cancer, wherein the antiresorptive agent is administered prior to or during the radiation therapy or prior to and during the radiation therapy.

31. A method of treating cancer in a subject comprising:
a) administering to the subject an amount of an antiresorptive agent effective to prevent or reduce radiation-induce loss of bone mass, bone density or bone strength; and
b) administering to the subject an amount of anti-cancer drug effective to treat the cancer.
